(19) United States
(12) Patent Application Publication
(10) Pub. No.: US 2024/0060990 A1
(43) Pub. Date: Feb. 22, 2024

(54) SPATIAL SIGNALING NETWORKS FOR MULTIPLEXED DRUG RESPONSE MEASUREMENT AND MODELING

(71) Applicant: Georgia Tech Research Corporation, Atlanta, GA (US)

(72) Inventors: Ahmet F. Coskun, Atlanta, GA (US); Shuangyi Cai, Atlanta, GA (US); Thomas Hu, Atlanta, GA (US)

(21) Appl. No.: 18/452,178

(22) Filed: Aug. 18, 2023

Related U.S. Application Data
(60) Provisional application No. 63/399,427, filed on Aug. 19, 2022.

(51) Int. Cl. G01N 33/68; G01N 21/64; C12Q 1/682; G01N 33/53; G01N 33/542

(52) U.S. Cl. CPC G01N 33/6872; G01N 21/6428; G01N 21/6458; C12Q 1/682; G01N 33/5308; G01N 33/542; G01N 2021/6439; C12Q 1/6804

(57) ABSTRACT

Disclosed herein are methods of detecting multiple protein interactions within a cell or tissue sample. This can be done by multiplexed imaging. Also disclosed are systems and kits for carrying this out.

### GOVERNMENT SUPPORT CLAUSE

This invention was made with government support under Grant No. P50CA217691, awarded by National Institutes of Health. The government has certain rights in the invention.

### BACKGROUND

Combination therapies have the potential to overcome acquired resistance to drugs. While single-cell RNA-seq, flow cytometry, mass cytometry, and western blots have provided cell compositions and drug responses of signaling proteins to study acquired resistance, subcellular and in-situ cellular distribution of physical signaling events and interactions are inefficiently captured in single cells due to the lack of spatial context of individual cells. Rationally designed combination therapies would necessitate an emerging validated multiplexed signaling assays directly in biological samples under drug perturbations.

Signaling networks are emerging approaches to studying the cellular alterations and decision-making in cancers. While the RNA expression data have suggested indirect networks of signaling proteins in many blood and solid cancers, single-cell mass cytometry, and phospho-flow have revealed direct networks of major signaling pathways and cascades at the protein level. However, these approaches demonstrate the signaling activity in cell suspensions without the location information about where cells reside and what their neighbors are in tissues and organs. In the meantime, image-based cell reports have been barcoded to dissect the signaling networks in live cells. These signaling network concepts achieve multiplexing of 100 signaling events, but they do not apply to fixed cells and tissues clinically used in studying signaling responses. Thus, multiplexed imaging methods are needed to visualize signaling networks even in archival formalin-fixed paraffin-embedded (FFPE) and fresh frozen patient tissues.

Current methods to analyze signaling pathways and receptor distributions include RNA sequencing and proteomics assays. However, this trend generally averages cellular information, causing loss of granularity due to single-cell heterogeneity. Flow cytometry and single-cell mass cytometry have then provided alternative approaches for single-cell analysis of signaling states and surface receptors; however, each cell's spatial information is lost due to the dissociation of the cell during measurements. Most molecular imaging methods have been limited to detecting a few signaling markers due to devices' spectral limitations, making it a challenge to visualize subcellular structures but with a limited number of biomarkers or signaling proteins. Thus, single-cell analysis of signaling mechanisms would benefit from emerging highly multiplex RNA and protein profiling technologies to decipher multiple signaling pathways. Spatial genomics assays have utilized image-based RNA barcoding based on multiplexed sequential fluorescence in-situ hybridization (FISH), providing ligand-receptor interactions in spatially resolved data. However, multiplexed RNA signatures would still suffer from indirect signaling networks. Imaging mass cytometry (IMC) and multiplexed ion beam imaging (MIBI) quantify up to 36 proteins using isotope-labeled antibody libraries imaged by specialized equipment. Besides, high-dimensional fluorescence imaging maps up to 50 proteins using DNA-barcoded imaging (CODEX), multiplexed fluorescence microscopy (MxIF), cyclic and sequential immunofluorescence techniques. These methods typically require specialized reagents and time-consuming labeling procedures, making them limited to research labs for discovery purposes.

Spatially resolved multiplexed imaging methods (IMC, MIBI, CODEX, and CyCIF) provided surprising single-cell heterogeneity in tissues with low-resolution imaging of 0.5 to 1-μm. Even cyclic immunofluorescence (CyCIF) needs biological samples to be manually removed from the sample holders, followed by staining outside the microscope and then placing the specimen back to the microscope for imaging. While protein-protein interaction (PPI) networks have yielded bulk level signaling interactions, automated and rapid multiplexed imaging-based direct signaling protein mapping technologies are needed to visualize the signaling factor colocalizations and subcellular protein interactions in single cells at high-resolution optical resolution.

These needs and others are at least partially satisfied by the present disclosure.

### SUMMARY

Disclosed herein is a molecular screening platform for imaging signaling proteins of human cell cultures and tissues from physiologically relevant cancer models using multiplexed measurement techniques to record spatial patterning of subcellular and cellular distributions in patients' biopsies with gene mutations. The biological lung cancer model system demonstrates how subcellular wiring of signaling factors provides subset patients that can be used for personalized and precision signaling therapies in lung cancers. This provides an advanced multiplexed protein imaging method to provide rapid experiments, automated microfluidics-based re-labeling and removal of signal, and imaging on the same platform. Here, a rapid multiplexed immunofluorescence (RapMIF) method is developed to visualize many molecules (30 to potentially 100 parameters) at high optical resolution. Protein interaction (RapMPI) assays are also used to resolve signaling networks of protein-protein interactions (PPIs) in cell cultures and tissues.

In an aspect, provided is a method of detecting multiple protein interactions within a cell or tissue sample, the method comprising: a) applying to the cell or tissue sample at least a first targeting moiety and a second targeting moiety, wherein the first targeting moiety interacts with a first specific protein of interest and the second targeting moiety interacts with a second protein of interest in the cell or tissue sample, wherein an interaction between the first specific protein of interest and the second specific protein of interest causes the first targeting moiety and second targeting moiety to be in close proximity to each other, wherein when the first targeting moiety and the second targeting moiety are in close proximity to each other, at least one detectable signal is produced; b) imaging the detectable signal or signals in the cell or tissue sample; c) deactivating the detectable signal or signals; d) repeating steps a), b), and c), wherein the first targeting moiety and the second targeting moiety are different in each repetition.

In another aspect, provided is a method of detecting subcellular spatial protein networks in a cell or tissue sample, the method comprising: a) applying to the cell or tissue sample at least one imaging moiety which can interact with a specific protein of interest in the cell or tissue sample, wherein when the at least one imaging moiety interacts with a specific protein of interest, it produces a detectable signal; b) imaging the detectable signal or signals in the cell or tissue sample; c) deactivating the detectable signal or signals; d) repeating steps a), b), and c), wherein the at least one imaging moiety is different in each repetition.

In another aspect, provided is a method of determining a signaling response in a cell or tissue, the method comprising: a) obtaining data on multiple protein interactions and subcellular spatial protein networks from a plurality of cell and tissue samples; b) analyzing the data using a machine learning algorithm; and c) using a neural network to create a signaling response model to an event in the cell or tissue.

In another aspect, provided is a system for multiplexed imaging comprising: a computer program capable of receiving at least one parameter from a user; a support for a cell or tissue sample; at least one port for delivering fluids to the support for the cell or tissue sample; and at least one port for aspirating fluids from the cell or tissue sample.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts a subcellular protein interaction assay for Cyclin E-CDK2 (PPI1) with Akt-phophoAkt (PPI2) in fresh PDX tumors, PC9 cultures, and EGFRm human tissues. Proximity ligation assay was used to detect subcellular positions of Cyclin E/CDK2 and Akt/phospho-Akt protein interactions (red dots) using 3 cycles. Unlabeled channel shows only cell background. Due to high fluorescence signal. Cycle 3 denotes the IF for CK+ (magenta) for mouse and human lung tumors cells. Concanavalin A labels PC9 cells.

FIG. 2 depicts spatial Akt-pAkt protein interaction in fresh human lung tissues. RapMPI works in fixed fresh tissues. Cycle 1 is PLA staining, after cycle 1 is PLA removal using DNase I, cycle 2 is pankeratin and B actin immunofluorescence staining. Left panels: Smwaller regions from whole slides in the right.

FIGS. 3A-3B depict spatial signaling velocity from RapMPI. FIG. 3A shows three cycle data generates A-B, C-D, and E-F PPIs in the same cell. The priori information on signaling cascades traced by arrow fields. FIG. 3B shows signaling velocity denoted by arrows from receptors to nucleus.

FIGS. 4A-4E depict rapid multiplexed immunofluorescence (RapMIF) for subcellular spatial protein analysis. FIG. 4A shows a schematic setup of a Rapid, automated, and multiplexed imaging system. The samples were stained with multiple cycles of IF using an autostainer. Created with BioRender.com. In FIG. 4B, the left panel indicates the activated Wnt and AKT signaling pathways, the markers highlighted in red were included in our panel. The right diagram demonstrates the predicted distribution among these proteins. Created with BioRender.com. FIG. 4C shows predicted expression of signaling markers in PC9 cells from the multiplexed protein data. Created with BioRender.com. FIG. 4D shows a schematic of the tissue samples stained with pan-cytokeratin used for tumor and stroma analysis of spatial signaling networks. Created with BioRender.com. FIG. 4E shows an illustration of the data processing and analysis pipeline. Multiplexed images provided the nuclear and cytosolic masks in A549 cells, correlation plot, protein prediction, the p-EGFR spatial intensity plot, and the pixel clustering maps of an individual PC9 cell. Created with BioRender.com.

FIGS. 5A-5F depict expression profiles and correlation analysis of multiplexed signaling proteins in A549 cells. FIG. 5A shows the correlation analysis pipeline of protein intensity at the single-cell level. Created with BioRender.com. FIG. 5B shows raw staining images in A549 cells from 23 protein markers and DAPI from 11 cycles. FIG. 5C shows a workflow of data analysis. The raw images were segmented and masked using WGA, Concanavalin A, WNT1, APC, and p-EGFR. The signaling intensity was quantified based on the cytoplasmic and nuclear masks. FIG. 5D shows the normalized mean intensity per cell of 25 markers for one ROI. 63 regions of interest, a total of 3457 numbers of A549 cells were analyzed. FIG. 5E shows pairwise marker correlation based on single-cell mean intensity in A549 cells was evaluated using Pearson Correlation and average linkage method on euclidean distance. The p-value of 0 for each pairwise correlation. FIG. 5E shows pairwise marker correlation based on single-cell mean intensity in the cytoplasm and nucleus-dependent manner was evaluated using Pearson Correlation using average linkage method on euclidean distance from the same cell as D. p-value of 0 for each pairwise correlation.

FIGS. 6A-6H depict spatial signaling network analysis in single pixels and prediction of phospho-proteins in A549 cells. FIG. 6A shows a pixel clustering analysis pipeline. Created with BioRender.com. FIG. 6B shows spatial signaling maps of 33 clusters at the single-pixel level. The UMAP representation of the distribution of these clusters in A549 cells was shown. The legend showed the cluster name in a frequency-descending order for each cell. FIG. 6C shows a heatmap of 33 clusters on a z-score scale. Each cluster represented one distinct expression profile of 21 protein markers in A549 cells. FIG. 6D shows pixel-level clustering of signaling proteins in one ROI in A549 cells. The highly expressed signaling proteins of different clusters were indicated in each cancer cell. FIG. 6E shows the importance of 20 protein markers in A549 cells was evaluated based on the Random Forest algorithm model Mean Decrease in Impurity (MDI). The MDI was defined as the total decrease in node impurity (weighted by the probability of reaching that node averaged over all trees of the ensemble in the Random Forest algorithm. FIG. 6F shows the expression level of p-β-catenin in A549 cells was predicted based on 20 features (R=0.75, p-value <0.001). FIG. 6G shows a comparison between the intensity level of the raw p-β-catenin and the predicted intensity in one ROI was obtained based on Random Forest analysis in A549 cells. FIG. 6H shows the quantification of cyclin D1 expression across DAPI expression level per cell in A549 cells. Most of the cells were harvested in the G1 phase. The right images show the comparisons of the cyclin D1 expression level in three cells in three different phases. Cell number 25 has a relatively higher cyclin D1 intensity than cell 23 and cell 28.

FIGS. 7A-7G depict the effect of osimertinib on signaling expression profiles and correlation analysis in PC9 cells. FIG. 7A shows raw staining images in PC9 cells from 23 protein markers and DAPI from 12 cycles. The cells were incubated in cell media for 48 hours and seeded on coverslips for multiplexing IF. n=531 number of cells were utilized. FIG. 7B shows raw staining images in PC9 cells from 23 protein markers and DAPI from 12 cycles. The cells were treated with 40 nM osimertinib for 48 hours and seeded on coverslips for multiplexing IF. n=451 number of cells were used. FIG. 7C shows the correlation of the single-cell mean intensity of 23 markers for control PC9 was evaluated using Pearson Correlation using the average linkage method based on Euclidean distance. FIG. 7D shows the correlation of the single-cell mean intensity of 23 markers for PC9 cells treated with 40 nM osimertinib for 48 hours was evaluated using Pearson Correlation based on the average linkage method implemented on Euclidean distance. FIG. 7E shows the p-EGFR (Tyr1086) intensity in PC9 cells across four-drug concentrations, 0, 20, 40, and 60 nM was analyzed and normalized to 0-tubulin. The cells were treated with drug or cell media for 48 hours and seeded on coverslips. The cells were stained with p-EGFR, and β-tubulin overnight at 4° C., followed by secondary antibody staining at RT 1 hour, 15 min DAPI staining. Stars indicate the statistical significance for pairwise comparison. P-value calculated using t-test independent samples with Bonferroni correction (***: 0.0001<p<=0.001, ****: p<=0.0001). FIG. 7F shows the comparison of expression profiles of 24 markers between control and 40 nM osimertinib samples. FIG. 7G shows the p-AKT(S473) intensity comparison between PC9 control and PC9 treated with 40 nM osimertinib multiplexing samples. The p-value was calculated using t-test independent samples with Bonferroni correction (****: p<=0.0001).

FIGS. 8A-8E depict the effect of osimertinib in spatial subcellular distribution and network in PC9 cells. FIG. 8A shows pseudo-colored cells by pixel clustering of 20 signaling protein markers in PC9 cells without osimertinib treatment. The legend indicates the cluster name in a frequency-descending order for each cell. FIG. 8B shows colored cells by pixel clustering of 20 signaling protein markers in PC9 cells with 48-hour 40 nM osimertinib treatment. The legend provides the cluster name in a frequency-descending order for each cell. FIG. 8C shows a heatmap of 20 clusters on a z-score scale. Each cluster represented one distinct expression profile of 20 protein markers in PCI cells. FIG. 8D shows an analysis pipeline for spatial networks. Created with BioRender.com. FIG. 8E shows spatial signaling networks of 19 clusters in control and 40 nM osimertinib-treated samples. Each node was labeled with one of the most expressed signaling markers (excluding the epigenetic markers) in that cluster of pixels. The color of the node matched with the cluster color shown in FIG. 8C, the color of the line represented the probability of neighboring with another cluster, and the size of the node provided the pixel distribution across all clusters.

FIGS. 9A-9E depict expression and spatial signaling analysis on tissue microarray from diverse lung cancers. FIG. 9A shows multiplexed protein images of lung tissue samples at normal, malignant stages IB, IIA, and IIIA. The tissue was masked based on pan-cytokeratin-positive staining. FIG. 9B shows the normalized total intensity of each marker per cell in pan-cytokeratin-negative (False) and -positive (True) regions. Stars indicated the statistical significance for pairwise comparison. P-value was calculated using t-test independent samples with Bonferroni correction (ns: p>=0.05, ****: p<=0.0001). FIG. 9C shows the comparison of signaling expression profiles of 24 markers. The tissue cores were classified by stages and pan-cytokeratin expression. Classification of signaling maps was demonstrated for 21 patients and 55 tissue cores using the average linkage method based on Euclidean distance to cluster cores and markers along the x and y-axis. FIG. 9D shows a heatmap of 19 clusters on a z-score scale was shown. Each cluster represented one distinct expression profile of 17 protein markers in lung microarray, excluding segmentation and epigenetic markers. FIG. 9E shows multiplexed signaling protein images from tissue cores were analyzed by single-cell level clustering of 17 protein markers in four stages of tumor. The expression profile was clustered in pan-cytokeratin-positive regions. Each stage contained three images from the same patient.

FIGS. 10A-10D depict key steps and timeline for the RapMIF. Related to STAR Methods. FIG. 10A shows a schematic illustration of the key steps in rapidly run cyclic immunofluorescence staining. A549 Cells are seeded on coverslips overnight, and PC9 cells are seeded and treated with the osimertinib drug for 48 hours. Following incubation, the cells are fixed and permeabilized using 1.6% Formaldehyde, and 0.5% Triton X-100 in PBS respectively. The cells are ready for staining with one set of pre-conjugated antibodies. The auto-stainer setup was shown in part 4. Following fluorophores deactivation, the cells are ready for the next round of staining and imaging. Created with Biorender.com. FIG. 10B shows an illustration of the microscope stage setup. Created with Biorender.com. FIG. 10C shows a timeline for the RapMIF. The cells on the coverslip are incubated with a cell staining medium for 1 hour and then stained with one set of antibodies conjugated with dyes and DAPI. Following imaging, the dyes on antibodies are bleached out using 3% H₂O₂ and 20 mM NaOH in 1×PBS solution, and the sample is ready for the next round of blocking, staining, and imaging. In between subsequent cycles, the dyes for antibody labeling were bleached and the same sample was re-blocking and stained with another set of antibody-dye conjugates. Created with Biorender.com. FIG. 10D shows an illustration of a cooled stage for overnight incubation of samples at 4° C. The tissue could be mounted on a coverslip to be compatible with the RapMIF system. using a thermal control system. Created with Biorender.com.

FIG. 11 depicts immunofluorescence images of 11 cycles of signaling markers in A549 cells. Related to STAR Methods. The figure shows the results in the same region of interest, including images before and after bleaching.

FIGS. 12A-12C depict bleaching analysis. Related to STAR Methods. FIG. 12A shows raw IF images of p-AKT-488, Cyclin E-555, and EGFR-647 at three conditions, stained with DAPI only, stained with targeted marker, and post-bleaching. The A549 cells were stained with conjugated antibodies for 1 hour at room temperature. FIG. 12B shows quantification of the signaling levels of p-AKT-488, p-AKT-555, and p-AKT-647. P-value calculated using Wilcoxon Rank Sum Test with Bonferroni correction (ns: 0.05<p, *: 0.01<p<=0.05, **: 0.001<p<=0.01, ***: 0.0001<p<=0.001, ****: p<=0.0001) Data are represented as mean expressions per cluster with 95% confidence interval. FIG. 12C shows quantification of single-cell marker intensity with comparison to after bleach intensity for PC9 cells. P-value calculated using Wilcoxon Rank Sum Test (ns: 0.05<p, ****: p<=0.0001). Box plot showing the distribution of the data with the minimum, first quartile (Q1), median, third quartile (Q3), and maximum.

FIG. 13 depicts raw staining images of 25 protein markers and DAPI from 12 cycles in PC9 cells. Related to STAR Methods. The cells were incubated in cell media for 48 hours and seeded on coverslips for multiplexing immunofluorescence.

FIGS. 14A-14C depict subpopulation classification in PC9 cells. Related to STAR Methods. FIG. 14A shows a UMAP of single PC9 cell phenotypes with both control and drug-treated (osimertinib) dataset. In FIG. 14B, the UMAP illustrates 12 subpopulations of PC9 cells. The cells were clustered based on the single-cell mean intensity (n=531 for control, n=451 for drug). FIG. 14C shows the heatmap demonstrates the signaling profiles for 12 subpopulations in PC9 cells.

FIGS. 15A-15D depict spatial analysis of MSCs. Related to STAR Methods. FIG. 15A shows the pixel clustering of four signaling markers in MSCs in two fields of view. FIG. 15B shows example MSCs of pixel clustering of 4 protein markers. The legend shows the cluster name in a frequency-descending order for each cell. FIG. 15C shows a heatmap of 10 clusters in z-score scale. Each cluster represents one distinct expression profile of 4 protein markers in MSCs. Data are represented as mean expressions per cluster. FIG. 15D shows a UMAP of 10 clusters, matching with a heatmap in FIG. 15C.

FIGS. 16A-16B depict a schematic illustration of RapMPI for subcellular spatial signaling networks. FIG. 16A shows a sketching of RapMPI combined with RapMIF in cell cultures or tissues. Multiple PPIs were detected using single-color PLA detection, followed by RapMIF to visualize proliferation, signaling, and organelles' markers. FIG. 16B shows a schematic showing the three distinct cell treatment prediction pipeline (1) count using machine learning model (2) Multi-Instance learning at the cell level with multi-layer perception network (3) the proposed spPPIGCN network that utilized single-cell spatial PPI graph for prediction.

FIGS. 17A-17E depict PPI networks, co-expression analysis, and predictive models of 16-plex profiling for 5 PPIs and 6 signaling and organelle markers in HCC827 cells. FIG. 17A shows the large FOV demonstrates the network analysis of five PPIs across five cycles in HCC827 cells without treatment and with 100 nM Osimertinib treatment for 12 hours. The total cell number is 950 for untreated and 560 for treated cells. The second column images show examples of networks in individual cells. Each node presents on the PPI event, and Delaunay triangulation has been performed to connect nodes. The third column images show the distribution of 5 PPIs in raw images, and the red outline is the p-EGFR. The cell boundary in magenta is p-EGFR IF staining in the fourth column images. FIG. 17B shows the quantification of PPI counts in HCC827 cells treated with and without Osimertinib. Statistical testing was performed using Mann Whitney Wilcoxon Test (***: 0.0001<p<=0.001, ****: p<=0.0001). FIG. 17C shows a workflow of data analysis. The expression and spatial distribution of protein markers, as well as the localization of PPIs, were detected using RapMPI and RapMIF. The mean expression of these proteins in each PPI neighboring window with a size of 5 in radius was calculated. FIG. 17D shows normalized co-expression of five PPIs with the mean intensity of six protein markers in single cells. The image on the right shows one example of the co-expression of ki67 and Cyclin E/cdk2 PPI in an individual cell. FIG. 17E shows 7 machine learning models to predict the treatment with the input of PPI counts in cellular (whole cells) or subcellular regions (cells separated by cytosol or nuclei).

FIGS. 18A-18C depict predictive models in 2D and 3D of 5 PPIs in HCC827 cells.

FIG. 18A shows a 3-Dimensional PPI scatter plot at the single-cell level in HCC827 cells. FIG. 18B shows a comparison of predictive model performance between 2D PPI counts and 3D PPI counts. The models have been performed on the PPIs distributed in whole single cells (top panel) or cells separated by cytosol and nuclei (bottom panel). FIG. 18C shows graph pooling of GCN model incorporating the PPI network. The performance has been compared between 3D and 2D PPIs. More comparisons of ML and DL models is in FIGS. 24A-24B.

FIGS. 19A-19F depict quantification, Co-expression, and modeling of 26-plex profiling for 9 PPIs and 8 signaling and organelle markers in HCC827. FIG. 19A shows a visualization of five PPIs overlayed with p-EGFR and DAPI. Images on the left are from large FOV, while PPI distributions in a single cell were displayed on the right. The total cell number is 723 for untreated and 605 for treated cells. FIG. 19B shows the network analysis of 9 PPIs across 7 cycles in HCC827 cells without treatment and with 100 nM Osimertinib for 12 hours in a large FOV (first panel) and individual cells (second panel). Each node presents on the PPI event, and Delaunay triangulation has been performed to connect nodes. The cell boundary in red is p-EGFR IF staining. The third panel illustrates the PPI distributions from the raw image. The last panels show the overlay of signaling, proliferation, and organelles markers. FIG. 19C shows the comparison of PPI counts in HCC827 cells between those treated with and without Osimertinib. Statistical testing was performed using Mann Whitney Wilcoxon Test (***: 0.0001<p<=0.001, ****: p<=0.0001). FIG. 19D shows one example of co-expression of Cox IV and Bim/Tom20 PPI in an individual cell. FIG. 19E shows normalized co-expression of 9 PPIs with the mean intensity of 8 protein markers in single cells filtered by COX IV positive regions. FIG. 19F shows 7 machine learning models to predict the treatment with the input of 9 PPI counts in cellular (whole cells) or subcellular regions (cells separated by cytosol or nuclei).

FIGS. 20A-20B depict visualization and quantification of PPI motifs from 5 PPI and 9 PPI networks. Quantification of the frequency of PPI motifs in HCC827 untreated and treated cells. Each number represents a unique motif from the 5 PPI (FIG. 20A) and 9 PPI network (FIG. 20B). Each node represents one unique PPI. Several examples of motifs in single cells were displayed in the figure. Statistical testing was performed using Mann Whitney Wilcoxon Test (***: 0.0001<p<=0.001, ****: p<=0.0001).

FIGS. 21A-21E depict quantification and modeling of 16-plex profiling for 5 PPIs and 6 organelles signaling markers in HCC827 cell-derived mouse xenografts. FIG. 21A shows a visualization of five PPIs followed by 6 proteins IF staining in HCC827 cell-derived mouse xenografts, treated with 1 week- and 2 month-Osimertinib separately. FIG. 21B shows a schematic showing the nearest-pixel method for assigning PPI signals to the nearest cell with incomplete cell segmented regions. FIG. 21C shows PPI quantification comparison in pan-cytokeratin positive regions between mice with 1-week and 2-month Osimertinib. Statistical testing was performed using Mann Whitney Wilcoxon Test (***: 0.0001<p<=0.001, ****: p<=0.0001). FIG. 21D shows an illustration of the PPI network in tissues at the subcellular level in two FOVs. FIG. 21E shows six machine learning models to predict the treatment outcome with the input of 5 PPI counts in whole cells or cells separated by cytosol or nuclei.

FIGS. 22A-22B depict a schematic design of PLA and the timeline for the RapMPI on cell cultures. FIG. 22A shows a schematic pipeline for detecting one PPI using PLA. FIG. 22B shows the cells are incubated with blocking buffer for 1 hour at 37° C. and then stained with one pair of antibodies conjugated with oligonucleotides overnight at 4° C. Following ligation (30 mins, 37° C.), amplification (100 mins, 37° C.), and DAPI staining (10 mins, RT), the signals can be imaged using a fluorescent microscope. Following imaging, the oligos and dyes on antibodies are removed using DNase I for 4 hours at RT. The sample is ready for the next round of blocking, staining, and imaging.

FIG. 23 depicts a PLA workflow for staining ten PPIs per cycle. In each cycle, the sample is incubated with 10 pair of proteins, and between cycles, the oligos and dyes are removed using DNase. The signals can be detected using a multi-spectrum microscope.

FIGS. 24A-24B depict a comparison of MIL and spPPI-GCN model performance in predicting single-cell drug perturbation using 5 PPI dataset. FIG. 24A shows a comparison of MIL and spPPI-GCN model Accuracy, AUC, F1 using different graph pooling layer for feature embedding aggregation for single-cell drug perturbation prediction. FIG. 24B shows a comparison of 2D vs 3D graphs of spPPI-GCN model Accuracy, AUC, F1 using different graph pooling layer for feature embedding aggregation for single-cell drug perturbation prediction.

FIGS. 25A-25B depict the effect of Nuclease P1 and DMSO on removing PPI oligos and fluorophores. FIG. 25A shows a schematic figure shows that the cells were stained with two sets of PPIs using RapMPI, and one cycle of IF staining. Between cycles, the oligonucleotides and fluorophores were removed using different deactivation approaches. FIG. 25B shows HCC827 cells were stained with five different sets of PPIs individually in a 96-well plate. Nuclease P1, 2-hour DNase, and DMSO effectively removed the PPI signals, and the samples were able to be labeled with a new set of proteins. Phalloidin, Concanavalin A, and WGA were labeled to outlier cell boundaries.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

### Methods

In an aspect, provided is a method of detecting multiple protein interactions within a cell or tissue sample, the method comprising: a) applying to the cell or tissue sample at least a first targeting moiety and a second targeting moiety, wherein the first targeting moiety interacts with a first specific protein of interest and the second targeting moiety interacts with a second protein of interest in the cell or tissue sample, wherein an interaction between the first specific protein of interest and the second specific protein of interest causes the first targeting moiety and second targeting moiety to be in close proximity to each other, wherein when the first targeting moiety and the second targeting moiety are in close proximity to each other, at least one detectable signal is produced; b) imaging the detectable signal or signals in the cell or tissue sample; c) deactivating the detectable signal or signals; d) repeating steps a), b), and c), wherein the first targeting moiety and the second targeting moiety are different in each repetition.

As used herein, the term “protein interaction” refers to a physical contact between at least two proteins. In some aspects, the physical contact can be accomplished by one or more of: hydrophobic bonding, van der Waals forces, salt bridges, ionic interactions, electrostatic forces, hydrogen bonding, electron sharing, disulfide bonds, or other suitable intermolecular or intramolecular forces. In some aspects, a given protein interaction can exist for any length of time depending on the function of the protein interaction and the proteins involved. In some aspects, the function of a protein interaction can be electron transfer, signal transduction, membrane transport, cell metabolism, muscle contraction, cell signaling, or other biological functions.

As used herein, the term “targeting moiety” refers to any molecule or compound which binds only to a specific motif, functional group, or domain in a compound or molecule of interest. In some aspects, each targeting moiety binds to a specific protein of interest. In some aspects, a targeting moiety can leverage a physiologically occurring interaction, or a targeting moiety can be synthetically designed to elicit a specific interaction. In some aspects, a targeting moiety can comprise an antibody. In some aspects, a targeting moiety can further comprise an oligonucleotide. In some aspects, the oligonucleotide can be either covalently or non-covalently bound to the antibody. In some aspects, a part of a first oligonucleotide on the first targeting moiety is complementary to a part of a second oligonucleotide on the second targeting moiety. In some aspects, the first oligonucleotide is not complementary to the second oligonucleotide.

The first targeting moiety and the second targeting moiety being “in close proximity” to each other refers to the first targeting moiety and the second targeting moiety having a distance between them that allows a part of the first targeting moiety and a part of the second targeting moiety to have an interaction between each other that spans at least this distance. In some aspects, this interaction can include hydrophobic bonding, van der Waals forces, salt bridges, ionic interactions, electrostatic forces, hydrogen bonding, electron sharing, disulfide bonds, or other suitable intermolecular or intramolecular forces. In some aspects, the distance can be from about 1 nm to about 50 nm, including exemplary values of about 2 nm, about 3 nm, about 4 nm, about 5 nm, about 6 nm, about 7 nm, about 8 nm, about 9 nm, about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, and about 48 nm. In some aspects, the distance is equivalent to a space between the first targeting moiety bound to the first specific protein of interest and the second targeting moiety bound to the second specific protein of interest when there is an interaction between the first specific protein of interest and the second specific protein of interest.

As used herein, the term “detectable signal” refers to a chemical compound or complex that can be either directly or indirectly detected by visual or instrumental means. A detectable signal may produce a signal that can be detected, such as a fluorescent, chemiluminescent or radioactive signal. A detectable signal can also be detected visually, for example, colored dyes.

In some aspects, producing a detectable signal comprises: amplifying the oligonucleotides which are in close proximity to each other, wherein the oligonucleotides are amplified only when in close proximity to each other; and applying to the cell or tissue sample a fluorescent-labeled oligonucleotide, wherein the fluorescent-labeled oligonucleotide is complementary to at least a part of the amplified oligonucleotides; wherein the detectable signal comprises the fluorescent-labeled nucleotide coupled to at least a part of the amplified oligonucleotides. In some aspects, the step of amplifying the oligonucleotides further comprises a polymerase enzyme and/or a ligase enzyme. In some aspects, the step of amplifying the oligonucleotides further comprises the addition of an additional oligonucleotide. In some aspects, a first part of the additional oligonucleotide is complementary to a part of a first oligonucleotide on the first targeting moiety and a second part of the additional oligonucleotide is complementary to a part of a second oligonucleotide on the second targeting moiety. In some aspects, the step of amplifying the oligonucleotides comprises rolling circle amplification.

In some aspects, step a) takes from about 30 minutes to about 5 hours, including exemplary values of about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, and about 4.75 hours.

In some aspects, step b) is performed using brightfield microscopy, florescent microscopy, confocal microscopy, scanning electron microscopy, transmission electron microscopy, or any other suitable imaging platform.

As used herein, the term “deactivating” refers to a modification of any part of a targeting moiety, any part of a detectable signal, and/or any part of a protein of interest such that a detectable signal can no longer be detected. In some aspects, step c) comprises incubating the cell or tissue sample with a nuclease to remove any oligonucleotides. As used herein, the term “nuclease” refers to any enzyme that cleaves nucleotides. In some aspects, the nuclease is selected from DNase and Nuclease P1. In some aspects, step c) takes from about 30 minutes to about 5 hours, including exemplary values of about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, and about 4.75 hours.

In some aspects, the method further comprises a wash step after step a), step b), and/or step c). In some aspects, the wash step uses phosphate-buffered saline (PBS), water, formamide, ethylenediaminetetraacetic acid (EDTA), or other suitable buffers or solvents. In some aspects, the wash step is repeated with the same or different fluid.

In some aspects, steps a), b) and c) are repeated at least from about two times to about thirty times, including exemplary values of three times, four times, five times, six times, seven times, eight times, nine times, ten times, twelve times, fourteen times, sixteen times, eighteen times, twenty times, twenty-two times, twenty-four times, twenty-six times, and twenty-eight times. In some aspects, the first targeting moiety and the second targeting moiety being “different” in each repetition refers to either a different compound used for the first and/or second targeting moieties, or the same compound used for the first and/or second targeting moieties but applied to a portion of the cell or tissue sample that is different spatially or temporally.

In some aspects, step b) further comprises taking images of the cell or tissue sample at multiple spatial and/or temporal locations. As used herein, “multiple spatial locations” refers to multiple regions of the cell or tissue sample that vary in the x-direction, the y-direction, and/or the z-direction. In some aspects, the multiple spatial locations can cover the entire cell or tissue sample. In some aspects, the multiple spatial locations can cover only a portion of the cell or tissue sample. As used herein, “multiple temporal locations” refers to multiple points in time, wherein the multiple points in time can be spread out across minutes, hours, days, weeks, months, or years. In some aspects, the multiple points in time are spread out evenly. In some aspects, the multiple points in time are not spread out evenly.

In some aspects, the method further comprises analysis of the images using a machine learning algorithm. In some aspects, the method further comprises a step of detecting subcellular spatial protein networks in the cell or tissue sample.

In some aspects, the method further comprises: e) applying to the cell or tissue sample at least one imaging moiety which can interact with a specific protein of interest in the cell or tissue sample, wherein when the at least one imaging moiety interacts with a specific protein of interest, it produces a detectable signal; f) imaging the detectable signal or signals in the cell or tissue sample; g) deactivating the detectable signal or signals; h) repeating steps e), f), and g), wherein the at least one imaging moiety is different in each repetition.

As used herein, the term “subcellular spatial protein network” refers to the spatial and/or temporal organization of proteins in the cytoplasm and nucleus of a cell.

As used herein, the term “imaging moiety” refers to any compound, molecule, or complex that can be either directly or indirectly detected by visual or instrumental means. The imaging moiety may produce a signal that can be detected, such as a fluorescent, chemiluminescent or radioactive signal. The imaging moiety can also be detected visually, for example, colored dyes. In some aspects, the at least one imaging moiety comprises an antibody.

In some aspects, the antibody is directly labeled. As such, the antibody which can interact with the specific protein of interest is conjugated to the fluorescent dye before the antibody is applied to the cell or tissue sample. In some aspects, the antibody is indirectly labeled. As such, a first antibody which can interact with the specific protein of interest is not conjugated to the fluorescent dye, and a second antibody which can bind to the first antibody is conjugated to the fluorescent dye before either antibody is applied to the cell or tissue sample, and the second antibody is applied to the cell or tissue sample after the first antibody is applied to the cell or tissue sample.

In some aspects, the step of deactivating comprises fluorescent bleaching. As used herein, the term “fluorescent bleaching” refers to rendering any fluorescent dyes on the cell or tissue sample undetectable. In some aspects, fluorescent bleaching is performed using a solution containing hydrogen peroxide (H₂O₂), sodium hydroxide (NaOH), or any other suitable bleaches or any combinations thereof.

In some aspects, the method further comprises a wash step after any one of steps e), f), or g).

In some aspects, steps e), f) and g) are repeated at least from about two times to about thirty times, including exemplary values of three times, four times, five times, six times, seven times, eight times, nine times, ten times, twelve times, fourteen times, sixteen times, eighteen times, twenty times, twenty-two times, twenty-four times, twenty-six times, and twenty-eight times. In some aspects, the imaging moiety being “different” in each repetition refers to either a different compound used for the imaging moiety, or the same compound used for the imaging moiety but applied to a portion of the cell or tissue sample that is different spatially or temporally.

In some aspects, subcellular spatial protein networks in the cell or tissue sample are detected.

In some aspects, at least one of the steps is performed using an automated system.

In another aspect, provides is a method of detecting subcellular spatial protein networks in a cell or tissue sample, the method comprising: a) applying to the cell or tissue sample at least one imaging moiety which can interact with a specific protein of interest in the cell or tissue sample, wherein when the at least one imaging moiety interacts with a specific protein of interest, it produces a detectable signal; b) imaging the detectable signal or signals in the cell or tissue sample; c) deactivating the detectable signal or signals; d) repeating steps a), b), and c), wherein the at least one imaging moiety is different in each repetition.

In some aspects, the imaging moiety comprises an antibody. In some aspects, the antibody is preconjugated with a fluorescent dye and the detectable signal comprises the fluorescent dye. In some aspects, the antibody is directly labeled. In some aspects, the antibody is indirectly labeled.

In some aspects, the step of deactivating comprises fluorescent bleaching. In some aspects, fluorescent bleaching is performed using a solution containing hydrogen peroxide (H₂02), sodium hydroxide (NaOH), or any other suitable bleaches or any combinations thereof.

In some aspects, the method further comprises a wash step after any one of steps a), b), or c).

In some aspects, step a) takes from about 30 minutes to about 5 hours, including exemplary values of about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, and about 4.75 hours. In some aspects, step c) takes from about 30 minutes to about 5 hours, including exemplary values of about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, and about 4.75 hours.

In some aspects, steps a), b) and c) are repeated at least from about two times to about thirty times, including exemplary values of three times, four times, five times, six times, seven times, eight times, nine times, ten times, twelve times, fourteen times, sixteen times, eighteen times, twenty times, twenty-two times, twenty-four times, twenty-six times, and twenty-eight times. In some aspects, the imaging moiety being “different” in each repetition refers to either a different compound used for the imaging moiety, or the same compound used for the imaging moiety but applied to a portion of the cell or tissue sample that is different spatially or temporally.

In some aspects, step b) further comprises taking images of the cell or tissue sample at multiple spatial and/or temporal locations.

In some aspects, the method further comprises analysis of the images using a machine learning algorithm. In some aspects, at least one of the steps is performed using an automated system.

In another aspect, provided is a method of determining a signaling response in a cell or tissue, the method comprising: a) obtaining data on multiple protein interactions and subcellular spatial protein networks from a plurality of cell and tissue samples; b) analyzing the data using a machine learning algorithm; and c) using a neural network to create a signaling response model to an event in the cell or tissue.

As used herein, the term “signaling response” refers to changes in the spatial and/or temporal organization of proteins and/or protein interactions in a cell or tissue sample to respond to a given event.

In some aspects, the data is obtained via any of the disclosed methods.

In some aspects, the machine learning algorithm is selected from data clustering, t-distributed stochastic neighbor embedding (tSNE), and uniform manifold approximation and projection (UMAP).

In some aspects, the machine learning algorithm is a convolutional neural network (CNN).

In some aspects, the event is administration of at least one therapeutic agent, a method of treatment, or onset of a disease. In some aspects, the method of treatment is exposure to radiation.

In some aspect, the method is used for screening a therapeutic agent, observing response to a therapeutic agent, observing response to a method of treatment, disease diagnosis, or stem cell therapy. In some aspects, the method of treatment is exposure to radiation.

### System

In another aspect, provided is a system for multiplexed imaging comprising: a computer program capable of receiving at least one parameter from a user; a support for a cell or tissue sample; at least one port for delivering fluids to the support for the cell or tissue sample; and at least one port for aspirating fluids from the cell or tissue sample.

In some aspects, each of the at least one port for delivering fluids and the at least one port for aspirating fluids further comprise a valve. In some aspects, each valve is controlled by the computer program.

In some aspects, the system can be coupled to a microscope. In some aspects, the microscope can be selected from a brightfield microscope, florescent microscope, confocal microscope, scanning electron microscope, transmission electron microscope, or any other suitable microscope.

In some aspects, the computer program executes any of the disclosed methods.

In some aspects, the at least one parameters are selected from: spatial position of the support; number of repetitions; time of the step of applying at least one detectable composition; number of images in the step of imaging; time of the step of deactivating; and/or wait time between any of the steps.

In some aspects, each port further comprises a fluidic tube and a needle tip. In some aspects, the system further comprises at least one port for preventing overflow using a vacuum.

### EXAMPLES

### Example 1a—Methods and Materials

Cells: A549 and PC9 cell lines were seeded on coverslips treated with 0.01% poly-l-lysine in a 6-well plate overnight, and cultured for 48 hours respectively at a 37° C. incubator. A549 was purchased from ATCC. The NSCLC-sensitive cell line, PC9, was provided by Dr. Sun Shi-yong (Emory University).

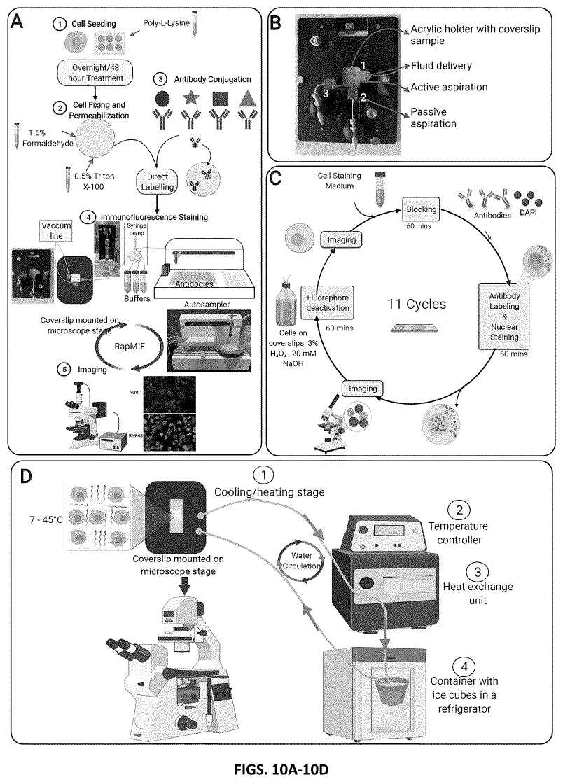

Drugs: The Osimertinib was provided by Dr. Sun Shi-yong (Emory University) at 10 mM concentration. The cells were treated with 0, 20, 40, and 60 nM Osimertinib for 48 hours in a 37° C. incubator. Following that, the cells were then fixed and permeabilized with 1.6% formaldehyde in 1×PBS for 10 minutes at RT and 0.5% Triton X-100 for 10 minutes at RT, respectively (FIG. 10A). The cells were then blocked using a cell staining medium (CSM) containing 5d. The effect of Osimertinib on p-EGFR (Tyr1086) and cell viability was tested. PC9 cells were stained with p-EGFR and β-tubulin overnight followed by 1-hour incubation of secondary antibodies at RT, and 15 mins DAPI staining. The cells were then imaged at 40× magnification, and the cell number was analyzed using ImageJ. For the multiplexing experiment, PC9 cells were treated with 40 nM Osimertinib for 48 hours in a 37° C. incubator, and then fixed, permeabilized, and blocked. The coverslip was mounted to an acrylic holder for multiple cycles of staining.

Tissues: FFPE lung cancer microarray tissue blocks were purchased from Biomax with 61 cores and 21 cases (BS04081a). The slide was baked in a 60° C. oven for 2 hours followed by deparaffinization and heat-induced epitope retrieval (HIER). Sectioned slices were ready for multiple rounds of staining, imaging, and bleaching.

Antibodies: A total of 26 antibodies were used. Cell segmentation included Phalloidin (A34055, Thermofisher Scientific), wheat germ agglutinin (WGA, W32466, Thermofisher Scientific), Concanavalin A (C11252, Thermofisher Scientific), β-tubulin (sc-5274, Santa Cruz Biotechnology), and β-actin (4970BF, Cell Signaling Technology). For tissue tumor region identification: pan-cytokeratin (914204, BioLegend) was used. 11 Markers targeting the WNT/β-catenin pathway included non-β-catenin (Ser45) (70034S, Cell Signaling Technology), APC (ab239828, Abcam), DKK1 (sc-374574, Santa Cruz Biotechnology), Cyclin E (sc-247, Santa Cruz Biotechnology), EMMPRIN (sc-21746, Santa Cruz Biotechnology), WNT1 (sc-514531, Santa Cruz Biotechnology), p-β-catenin (Ser45) (44-208G, Thermofisher Scientific), RNF43 (ab84125, Abcam), DKK2 (PA5-11616, Thermofisher Scientific), AXIN1 (2087BF, Cell Signaling Technology), Cyclin D1 (ab156448, Abcam). 6 Markers involved AKT/mTOR pathway included p-AKT (Ser473) (4060BF, Cell Signaling Technology), AKT (4691BF, Cell Signaling Technology), p-mTOR (Ser2448) (5536BF, Cell Signaling Technology), mTOR (2983BF, Cell Signaling Technology), p-EGFR (Tyr1086) (36-9700, Thermofisher Scientific), EGFR (sc-373746, Santa Cruz Biotechnology). 3 epigenetic markers included H3K27me3 (9733BF, Cell Signaling Technology), H3K9ac (ab203951, Abcam), H3k4me3 (ab227060, Abcam). For rapid staining of the sample, the BSA-free primary antibodies were pre-conjugated with Alexa Fluor 488, 555, or 647 using the Lightning-Link™ Rapid Conjugation kit (ab236553, ab269820, ab269823). Before conjugation, the concentrations of the antibodies were measured using Nanodrop to ensure the required antibody amount was in the range. The overall time for the conjugation process was around 20 minutes. 15 antibodies involved in signaling pathways were first tested in IF of A549 cells. For conjugated antibodies, IF was tested under four conditions, indirect/direct labeling, overnight 4° C./RT 1 hour, respectively. For antibodies conjugated to Alexa Fluor dyes, the direct labeling was tested at overnight 4° C./RT for 1 hour. To improve the staining quality, titrations were performed on antibodies, and IF was evaluated at two different dilution rates. The improved staining condition of each antibody was determined based on the SNR enhancement. The SNR of each condition was measured based on calculations of the max and min pixel values of five random cell regions in ImageJ (TABLE 1). For IF staining on cell culture, the antibodies and DAPI (62248, Thermofisher Scientific) were diluted in CSM containing 0.5% BSA, and 0.02% sodium azide in PBS. For IF staining on tissue, the antibodies and Hoechst 33342 (H3570, Thermofisher Scientific) were diluted in a protein block buffer (DAKO, X0909).

TABLE 1
SNRs comparison at different dilution
rates (related to STAR Methods).

| Marker | Dilution rate | SNR |
|---|---|---|
| p-AKT | 1:200 | 39.78 |
| | 1:800 | 28.42 |
| p-EGFR | 1:100 | 172.91 |
| | 1:400 | 23.15 |
| Axin1 | 1:200 | 36.70 |
| | 1:500 | 23.51 |
| Cyclin E | 1:200 | 1100.79 |
| | 1:500 | 424.81 |
| WNT1 | 1:100 | 14.31 |
| | 1:500 | 9.93 |
| EMMPRIN | 1:100 | 18.76 |
| | 1:500 | 17.05 |
| Non-p-β-catenin | 1:100 | 15.96 |
| | 1:400 | 3.97 |
| APC | 1:200 | 40.90 |
| | 1:500 | 32.01 |
| DKK1 | 1:100 | 1225.08 |
| | 1:500 | 29.78 |
| p-β-catenin | 1:400 | 21.52 |
| | 1:800 | 17.21 |
| RNF43 | 1:200 | 38.18 |
| | 1:500 | 18.18 |
| DKK2 | 1:100 | 27.64 |
| | 1:500 | 18.82 |

RapMIF: The RapMIF setup consisted of an autosampler that transferred antibodies and performed washes of the sample automatically and controlled by a custom-written python code (FIGS. 10A-10D). The buffers used during the procedure are 1×PBS (D8537, Sigma), CSM, and Fluorophore bleaching solution. PBS 1× was the wash buffer for the stabilization of the cells and the tubes. CSM was the imaging buffer that helped in the annealing of antibodies with the cells. Fluorophore bleaching solution was the strip buffer used to remove staining after each cycle. The walls on the 96-well plate for the antibodies were pre-washed with 1×PBS. The well plate was covered with foil stickers to ensure a dark condition. The microscope stage setup included tubes for transporting buffers and an acrylic holder for mounting cells on coverslips. The stage was designed to be compatible with the Keyence BZ-X810 microscope. The flow of the buffers was controlled by a valve. The stage included three 3D-printed ports, connecting fluidic tubes to needle tips. One port was used for delivering the fluids to the solution, while the other two function as aspirating fluids and preventing overflow using a vacuum (FIG. 10B). To mount the cells, the coverslip containing cells was placed on an adhesive spacer. It was attached to the acrylic holder using instant adhesive such that the cells were on the side facing the well. The acrylic holder was then secured onto the microscope stage. Once the cells were mounted and the antibodies and buffers were ready for transfer, automatic staining was done by running Python code. The code used the following as inputs: 1) position of the first well, 2) the number of imaging cycles, 3) time to wait after staining, 4) whether to bleach before staining, and 5) time to wait after bleaching. The default time for staining was 60 minutes. The default time for the bleaching step was 60 minutes. In between subsequent cycles, the dyes attached to the antibodies were bleached out using the fluorophore bleaching solution, followed by the re-labeling of a new set of three antibody-dye conjugates (FIG. 10C). The parameter settings including fluid transfer and antibody incubation for the RapMIF setup are shown in TABLE 2. The total time taken for one staining cycle was approximately 5 hours including imaging and bleaching steps. RapMIF advances the current CycIF approach by saving time spent on each cycle. RapMIF takes around 5 hours per cycle, while CycIF takes more than 12 hours per cycle (TABLE 3). Although another 1 hour for each cycle was spent for cell reblocking to avoid the nonspecific binding, the overall time of RapMIF is still shorter.

TABLE 2
List of parameters optimized in the Python code (related to STAR Methods).

| | | Default | |
|---|---|---|---|
| Parameter | Description | value | |
| firstWellLocation | Denotes the location of the first well on the 96-well plate that | 1 | |
| | should be considered for the first cycle during RapMIF | | |
| PrimaryTime | Time to wait for primary antibody incubation. | 60 | minutes |
| SecondaryTime | Time to wait for secondary antibody incubation. Can be | 60 | minutes |
| | commented out for directIF. | | |
| bleachTime | Time to wait for bleaching incubation | 60 | minutes |
| blockTime | CSM (Cell Staining Media) blocking time | 60 | minutes |
| washTime | Waiting time between PBS washes | 5 | minutes |
| aspirationTime | Active aspiration time to remove the fluids from the sample | 4.5 | seconds |
| MixVolume | Volume of diluted antibody drawn out by the needle from the | 250 | mL |
| | 96-well plate | | |
| wellMaxVolume | Maximum volume of the acrylic well | 900 | mL |

TABLE 3
Time comparison between CycIF and
RapMIF (related to STAR Method)

| | |
|---|---|
| CycIF | RapMIF |

| | | | | |
|---|---|---|---|---|
| Blocking | 1 | hour | 1 | hour |

| | | | |
|---|---|---|---|
| Staining | Overnight | 1 | hour (including Hoechst) |

| Hoechst staining | 15 | min | 0 | |
|---|---|---|---|---|
| Bleaching | 1 | hour | 1 | hour |

| | | | |
|---|---|---|---|
| Re-blocking | N/A | 1 | hour |

| | | | | |
|---|---|---|---|---|
| Total | >12 | hours | 5 | hours (include imaging) |

Cell multiplexing: Each coverslip prepared with cells was stained with 25 markers using a total of 11-12 cycles (FIG. 10C). The staining process followed the steps below: 1) block the sample with CSM for 1 hour at RT; 2) stain the sample with a set of conjugated-antibodies and DAPI diluted in CSM for 1 hour at RT followed by 3 times 1×PBS wash; 3) image the sample using Keyence fluorescence microscopy; 4) deactivate the fluorophores using 3% H₂O₂ and 20 mM NaOH made up in PBS for 1 hr at RT in the presence of white light, followed by 3 times 1×PBS wash; 5) reimage the same region of interest (ROI) of the sample to check the residuals after bleaching, following the step 1-5 for next round of staining. To quantify the frequencies belonging to the signal that disappears after bleaching, a Fourier analysis of before and after bleaching was performed to indicate the difference in frequencies belonging to the signal.

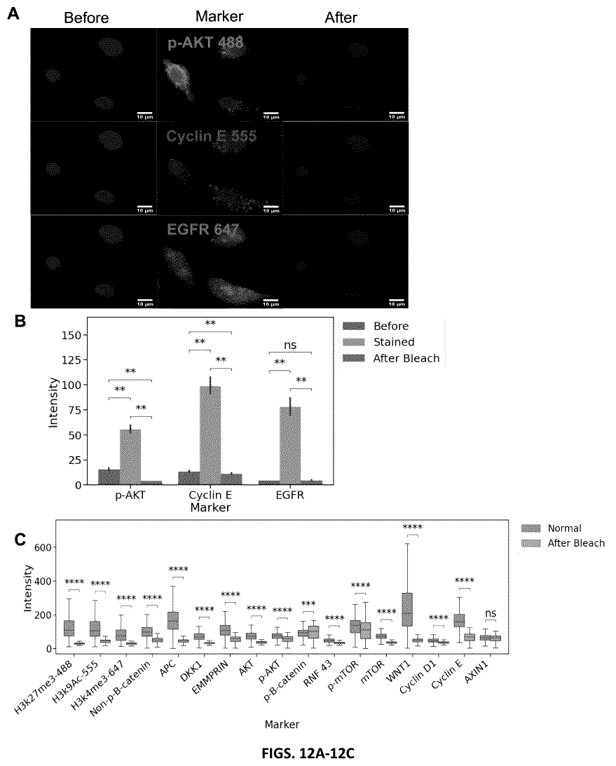

To examine the efficiency of bleaching, the signaling levels of p-AKT-488, Cyclin E-555, and EGFR-647 before and after bleaching were quantified (FIGS. 12A-12C). The A549 cells were stained with conjugated antibodies for 1 hour at room temperature. The sample was imaged in ten different regions. The signaling levels at three conditions are visualized in the histogram. For all three markers, the signaling levels of them returned back to the background level after 1-hour incubation of the bleaching solution. Bleaching for 60 mins should be sufficient to reduce the intensity to 10² to 10³-fold (Lin et al., 2018). The signals before staining, after staining, and after bleaching were quantified after background subtraction and applying a cell mask (FIG. 12B). Also, quantification of single-cell marker intensity with comparison to after bleach intensity for PC9 cells was performed to examine the efficiency of the bleaching step (FIG. 12C).

Tissue multiplexing: RapMIF is compatible with tissues mounted on coverslips and the tissue holder would be processed on a cooled stage holder (FIG. 10D). For antibody incubation on tissue, 4° C. can be performed without removing the sample from the microscope to ensure the same position of the sample for imaging. The temperature was controlled using a cooling and heating stage, a temperature controller, and a BTC-W heat exchange unit. The heat exchange unit uses liquid water to bring down the temperature of the stage during cooling. A tubing system was used for a closed-loop of water circulation. However, the commercial microarrays are available in the tissue slide format, necessitating a new pathological sample preparation pipeline to make microarrays on coverslips. While microarray on coverslip is feasible, here available tissue microarrays were used. Thus, for tissue specimens mounted on slides, to maximize the efficiency of detecting epitopes in tissue, the same protocol was followed as the previous t-CycIF protocols by using overnight incubation (Lin et al., 2018). The iterative process was modified in blocking and bleaching steps: after antigen retrieval, the sample follows the steps below: 1) block the sample with protein block buffer for 30 minutes at RT; 2) stain the sample with a set of conjugated-antibodies and Hoehst diluted in protein block buffer overnight at 4° C., followed by 3 times 1×PBS wash; 3) mount the sample in 10% glycerol made in 1×PBS, and cover the slide using 24×50 mm No. 1 coverslip (3322, Thermo Scientific) to prevent evaporation during imaging; 4) image the sample using Keyence fluorescence microscopy; 5) de-coverslip the sample by placing the slide in a vertical jar containing 1×PBS for around 10 minutes, and the coverslip was released due to gravity. 6) deactivate the fluorophores using 4.5% H₂O₂ and 24 mM NaOH made up in PBS for 1 hr at RT in the presence of white light, followed by 3 times 1×PBS wash; 7) followed by mounting, reimage the same ROI of the sample to check the residuals after bleaching, following the step 2-7 for next round of staining. After IF staining, H&E staining was performed at the last cycle.

Imaging: A wide-field microscope, Keyence BZ-X700, was used for fluorescence and brightfield imaging. For each cycle of imaging, four channels were captured with 40% excitation light: Channel 1 detects DAPI/Hoechst at 360 nm, Channel 2-4 detects fluorophores at Alexa Fluor 488 nm (FITC), 555 nm (TRITC), and 647 nm (Cy5), respectively. The exposure time was varied, but the exposure time for each marker across control and drug-treated PC9 cells was consistent. The exposure time for Channel 1, DAPI/Hoechst was around 1/400s, and Channel 2-4 varied from is to 1/20s. The sample for cells grown on the coverslip was imaged at a 40× oil lens for 48-62 region-of-interest (ROI), and each ROI was imaged across 25-30 z-stacks with 0.4 μm/stack. To determine the optimal step size for imaging, A549 cells stained with WNT1 marker were imaged at four different step sizes, 0.1-μm, 0.4-μm, 0.8-μm, and 1.2-μm. The step size of 0.4-μm was chosen for the imaging system to reveal more continuity of the dim signaling molecules. Even though the acquisition time was around 1 hour for each cycle, and slower compared to images at a larger step size, the signaling molecules could be better resolved. The 40× oil lens achieved a high-resolution acquisition at 0.18872 μm/pixel. The tissue microarray was imaged at a 20× dry lens, each ROI was imaged using autofocus. The 20× dry lens provided a resolution of 0.37742 μm/pixel. The best focus z level was determined by calculating the blur score of each DAPI channel image. The blur score of each image was calculated by taking the fast Fourier transform (FFT) transform of the image and filtering out low frequencies, the resulting inverse Fourier transform image's mean magnitude was calculated. All z-level images of one location were then ranked based on the mean magnitude as a blurriness score. The best focus images around +/−2 z-stacks were used for signaling analysis. 3D subcellular spatial signaling levels were also assigned into 26-pixel clusters in 3D across 21 z-stacks, demonstrating the 3D distributions of signaling proteins as a proof of concept.

Background correction: To correct the uneven illumination caused by the flat and dark field, a BASIC algorithm in the ImageJ plugin was used to remove the line artifacts in each tissue core (Peng et al., 2017). Since the patterns of the uneven illumination across different channels were different, and the shape and size of tissue cores varied, the correction was applied per tissue core for each channel separately across 12 IF cycles. The software estimated the flat and dark fields and subtracted the raw images from the two fields to generate corrected images. Then, images were post-processed for quantification of signaling networks and intensity levels.

Segmentation: For cell culture, single-cell nuclei regions were segmented using Cellpose (Stringer et al., 2021) by cell painting markers (Bray et al., 2016). The best focus images around +/−2 z-stacks were used for segmentation of the cells. DAPI or Hoechst marker was used to segment cell nuclei boundaries. DAPI and segmentation markers were used to register the cells across the multiple cycles. In the A549 experiment, the Phalloidin channel showed strong background noise due to the effect of the bleaching solution, therefore p-EGFR, Concanavalin A, B-actin, WGA, APC, and WNT1 were combined to outline the cell boundary for segmentation. For PC9 data, Phalloidin marker was used for cell boundary segmentation. For FFPE lung cancer microarray tissue, DAPI was used to segment the nuclei region. The cytosol region was calculated by expanding the nuclei segmented region by 10 pixels. The 10 pixels were determined empirically by looking at nuclei segmentation and cytosol segmentation markers.

Image intensity: From nuclei and cytosol cell masks, the mean intensity of markers in each cell area was calculated. Each cell nuclei and cytosol region were separated and mean intensity for each marker was calculated and cell masks with a colormap showing the intensity were generated to visualize the variation across signaling markers within and outside the nuclei area.

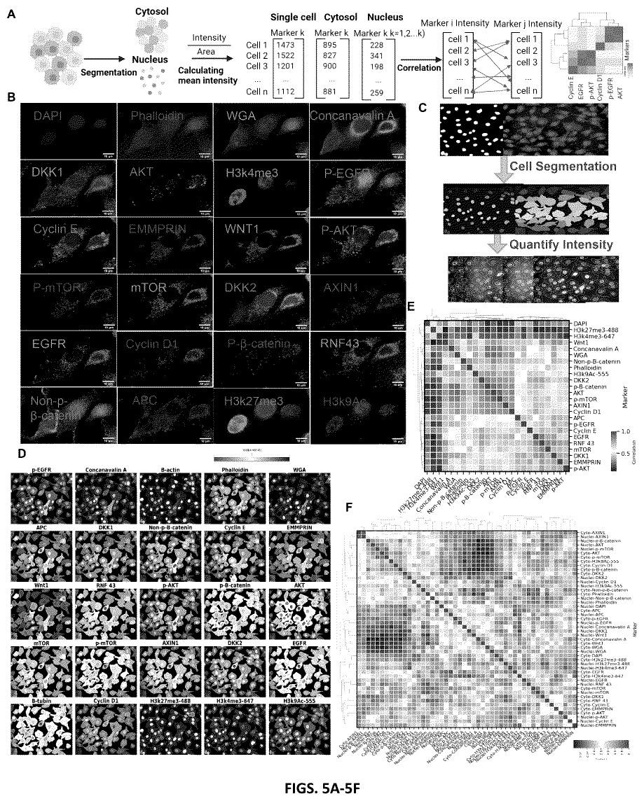

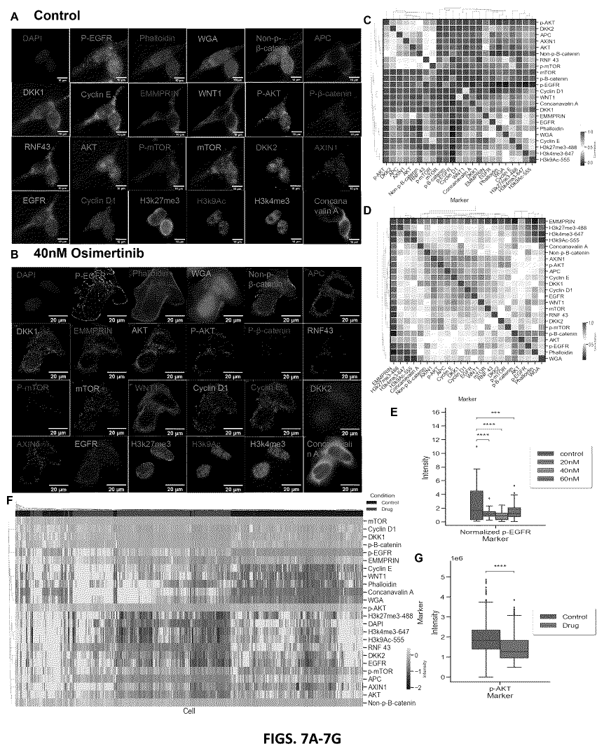

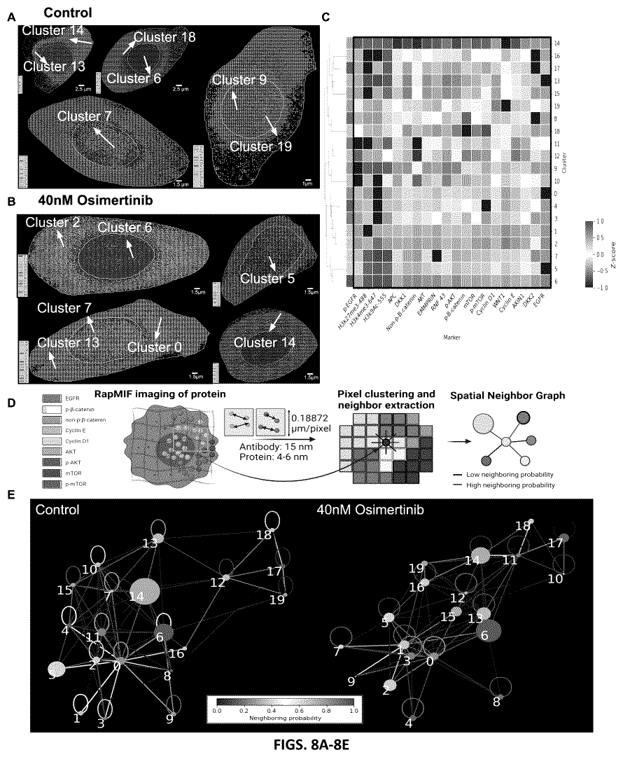

Correlation analysis: Correlation between pairs of cells was performed based on the mean intensity level for a pair of markers across all the cells (FIGS. 5A-5F). The correlation between pairwise markers at the cell level was investigated to identify co-expression patterns. For the correlation analysis, the Pearson correlation of pairwise markers at the single-cell level was quantified by using the mean intensity distribution for pairs of markers across the cells (FIGS. 5E-5F, FIGS. 7C-7D). The per cell mean intensity was first extracted for all markers to obtain a vector of mean intensities per cell. The nuclei and cytosol region were then separated for each cell and extracted one vector of mean intensities in the cytosol and another vector of mean intensities in the nuclei per cell. To confirm the bleaching efficiency, the mean intensity correlations of the before and after bleach images was compared. The 3-way correlation was calculated with the adjusted-R² value of the linear regression model of one marker predicted by the two others. Specifically, the expression of the maker on the z-axis was predicted by the two markers on the x and y-axis using a 3D scatter plot.

Marker intensity prediction: Per cell intensity of individual markers was predicted from other available markers using a Random Forest regressor with a 5-fold cross-validation training. The training set was composed of 2885 single-cell data points with a testing set of size 572. The regression was evaluated by the r-score between true intensity per cell compared to predicted intensity per cell. The r-value found was 0.76 with a p-value<0.01. Feature importance was plotted using the impurity index.

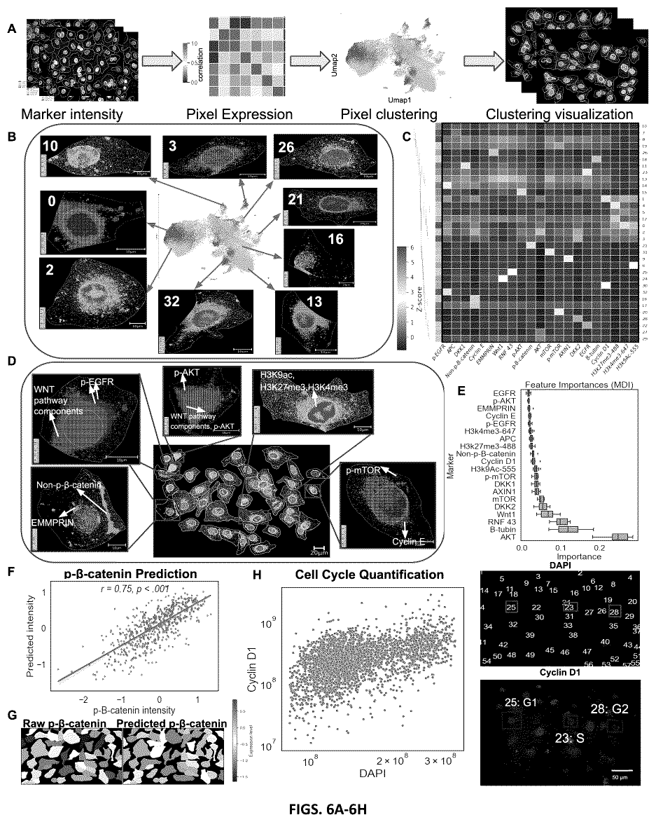

Pixel clustering: Pixel phenotypes were clustered in a two-step clustering pipeline (FIG. 6A). From each pixel location within the cell segmented region, the intensity value of each marker expression was extracted. The resulting feature matrix consisted of n rows of a total number of pixels and p columns equal to the number of markers. Each column of the feature matrix was min-max normalized. Then to filter out the background, each pixel location (row in the matrix) with all markers intensity lower than 0.3 was considered as background and dropped from the feature matrix. The cut-off of 0.3 was determined empirically so noise was filtered out of the cell segmented region. The feature matrix was first clustered using the K-Means algorithm with a high number of clusters (k=60). Then, the mean expression per cluster was calculated and the cluster was merged based on hierarchical clustering on the cosine similarity of the mean expression using the average linkage method with a tolerance of 0.2 of the maximum distance between clusters. The latent feature space was obtained using Uniform manifold approximation and projection (UMAP) and cluster labels were visualized by assigning each cluster color and reprojected on the UMAP embedding. A Heatmap of the mean expression level of marker per cluster was generated to show the variation of cluster phenotype.

Spatial signaling network: The network representation of spatial signaling was represented using the spatial information of pixel clustering. Each cluster was represented by a node with node size in the network proportional to the number of pixels in the corresponding cluster. For each cluster, the distribution of the neighbor cluster was calculated by looking at the 2-hop neighbor's cluster of all pixels in the cluster. The edge color represented low (blue) to high (red) neighboring probability between two clusters.

Subpopulation clustering: A549 and PC9 dataset subpopulations were defined based on signaling phenotype using unsupervised clustering with the Leiden algorithm from multiplex protein mean expression level per cell (Traag et al., 2019). Both A549 and PC9 datasets were clustered into 12 clusters.

Receptor discontinuity: The centroid of nuclei of each cell was calculated from each cell mask. Then each cell was divided into regions defined by the absolute distance from the centroid. In each region, the mean intensity of the marker was quantified as a function of distance from the nuclei centroid. Distances from nuclei centroid in each cell were shown as a radius map and overlaid with marker intensity with a custom colormap. The colormap for marker intensity was transformed from matplotlib package ‘greens’ with alpha set using the NumPy function linspace going from 0 to 1 with N equal to the number of RGB quantization levels in the colormap.

Cell survival: The effect of Osimertinib on PC9 cell proliferation and survival was measured using ImageJ. The cell survival was measured based on the cell number on the coverslip. The cells were seeded on coverslips at the same starting concentration, 0.833×10⁵, followed by 48-hour treatment of Osimertinib at 0, 20, 40, and 60 nM. The cells were stained with p-EGFR, and β-tubulin and three ROIs were imaged for each concentration at 40×. The cell number on each ROI was counted in ImageJ following the steps: import the Channel image, make binary, convert it to masks, analyze particles, and then choose “masks” under the “show” option.

Statistical testing: The details of statistical tests employed in each case were provided in the figure captions. All P values were corrected for multiple testing and the statistical testing method was indicated in the figure captions. The following convention was used to indicate significance with asterisks: not significant (ns) (P>0.1), * (0.1>P>0.01), ** (0.01>P>0.001), *** (0.001>P>0.0001), and **** (P<0.001).

### Example 1b—Methods and Materials

Cells: A549 cells were used for antibody optimization. The cells were seeded on coverslips treated with 0.01% poly-l-lysine in a 6-well plate overnight at a 37° C. incubator. A549 was purchased from ATCC. NSCLC-sensitive cell line, HCC827 was provided by Dr. Sun Shi-yong (Emory University). HCC827 cells were seeded on coverslips in a 6-well plate overnight, followed by Osimertinib treatment.

Drugs: The Osimertinib was provided by Dr. Sun Shi-yong (Emory University) at 10 mM concentration. HCC827 cells were treated with 100 nM Osimertinib at different time points in a 37° C. incubator. Following that, the cells were then fixed and permeabilized with 1.6% formaldehyde in 1×PBS for 10 minutes at RT and 0.5% Triton X-100 for 10 minutes at RT, respectively. The cells were then blocked using a cell staining medium (CSM) containing 0.5% BSA, and 0.02% Sodium Azide in PBS. The effect of Osimertinib on p-ERK(T202/Y204) was assessed. HCC827 cells were stained with p-ERK overnight followed by 1-hour incubation of secondary antibodies at RT, and 10 mins DAPI staining. The cells were then imaged at 40× magnification, and the intensity levels were analyzed. For the multiplexing experiment, HCC827 cells were treated with 100 nM Osimertinib for 12 hours in a 37° C. incubator, followed by RapMPI. The coverslip was mounted to an acrylic holder for multiple cycles of staining.

Tissues: HCC827-derived mouse xenografts were provided by Dr. Sun Shi-yong (Emory University). The mice were treated with Osimertinib for 1 week and 2 months respectively. Osimertinib was given to mice daily. Mice were then euthanized, and the tumor was embedded in OCT for sectioning. The OCT tissues were fixed in acetone, rehydrated, permeabilized in 0.4% Triton X-100, blocked, and ready for multiplexed experiments.

Antibodies: A total of 27 antibodies were used. 9 PPIs targeting the AKT/mTOR, MEK/ERK, and YAP/TEAD1 pathways included TEAD1 (12292BF, Cell Signaling Technology), YAP (ab172373, Abcam), Cyclin E (sc-247, Santa Cruz Biotechnology), CDK2 (sc-6248, Santa Cruz), p-ERK (T202+Y204) (ab242418, Abcam), c-Myc (5605BF, Cell Signaling Technology), p-AKT (Ser473) (4060BF, Cell Signaling Technology), mTOR (2983BF, Cell Signaling Technology), Mcl-1 (66157BF, Cell Signaling Technology), Bak (12105BF, Cell Signaling Technology), Cyclin D1 (66467, Cell Signaling Technology), CDK4 (23972, Cell Signaling Technology), NF-κB p65 (69994SF, Cell Signaling Technology), p-p90RSK (Ser380) (11989BF, Cell Signaling Technology), Bim (26184SF, Cell Signaling Technology), Tom20 (sc-17764, Santa Cruz), Oct4 (ab240358, Abcam), Sox2 (ab243909, Abcam). 10 of protein markers were used for RapMIF, including p-EGFR (Y1068) (ab205827, Abcam), Tom20 (sc-17764, Santa Cruz), Ki67 (ab283699, Abcam), Pan-cytokeratin (53-9003-82, Invitrogen), Golph4 (ab197595, Abcam), NBD-C6 (N22651, Thermofisher), Cox IV (ab197491, Abcam), Phalloidin (A34055, Thermofisher Scientific), wheat germ agglutinin (WGA, W32466, Thermofisher Scientific), and Concanavalin A (C11252, Thermofisher Scientific). For RapMIF, the antibodies were either purchased in preconjugated versions or conjugated with Alexa Fluor 488, 555, or 647 using the Lightning-Link™ Rapid Conjugation kit (ab236553, ab269820, ab269823). For RapMPI, the carrier-free antibodies were pre-conjugated with Duolink Probemaker (single color: DU092009 Sigma; multicolor: DU096020 Sigma). Before conjugation, the concentrations of the antibodies were measured using Nanodrop to ensure the required antibody amount was within the range. All antibodies involved in signaling pathways were first tested by IF in A549 cells. For unconjugated antibodies, IF was tested at overnight 4° C./RT 1-hour conditions. To improve the staining quality, titrations were performed on antibodies, and IF was evaluated at two different dilution rates. To optimize the antibody conditions, A549 cells were seeded on coverslips for antibody staining, followed by DAPI (62248, Thermofisher Scientific). For IF staining on tissue, the antibodies and Hoechst 33342 (H3570, Thermofisher Scientific) were diluted in a protein block buffer (DAKO, X0909).

RapMPI on cell cultures: For single color detection, each coverslip prepared with cells was stained with multiple PPIs with one PPI per cycle. The cells were fixed, permeabilized, and then ready for the following staining steps: 1) block the sample with Duolink blocking solution; 2) incubate it with one pair of proteins, conjugated to one PLUS and one MINUS oligonucleotide at 4° C. overnight. The antibodies were diluted in Duolink antibody diluent at the preferred dilution rate; 3) incubate the sample with ligase for 30 mins at 37° C.; 4) amplify the signals for 100 mins at 37° C.; 5) stain the sample with DAPI, and the sample was ready for imaging.

For multicolor detection, after permeabilization, the cells underwent the following steps: 1) block the sample with Duolink blocking solution; 2) incubate it with two pair of proteins, conjugated to one pair of oligonucleotides at 4° C. overnight. The antibodies were diluted in Duolink antibody diluent at the preferred dilution rate; 3) incubate the sample with ligase for 30 mins at 37° C.; 4) amplify the signals for 100 mins at 37° C.; 5) incubate the sample with the detection buffer for 30 mins at 37° C.; 6) stain the sample with DAPI, and the sample was ready for imaging. After imaging, the samples were incubated with DNase at a 1:50 dilution rate for 4 hours at RT, followed by 3 times 30% formamide washes and 3 times 1×PBS washes.

RapMPI on tissues: RapMPI is compatible with tissues mounted on slides. The staining settings were the same as the RapMPI in cell cultures. The blocking step between cycles for tissue multiplexing was skipped to avoid hiding the signals. The tissue samples were mounted with 10% glycerol made in 1×PBS and covered the slide using a 24×50 mm No. 1 coverslip (3322, Thermo Scientific) to prevent evaporation during imaging. To de-coverslip the sample after imaging, the slide was placed in a vertical jar containing 1×PBS for around 10 minutes, and the coverslip was released due to gravity.

RapMIF: Following RapMPI, RapMIF was performed to profile Pan-cytokeratin, ki67, Tom20, p-EGFR, Golph4, Bim, Concanavalin A, Phalloidin, and WGA. The settings were the same as described previously²⁰. Between cycles, the fluorophores were deactivated using and 3% H₂O₂ and 20 mM NaOH mixture made up in 1×PBS for 1 hr at RT in the presence of white light, followed by 3 times 1×PBS wash (for cell cultures: 3% H₂O₂ and 20 mM NaOH; for tissues: 4.5% H₂O₂ and 24 mM NaOH). For tissue multiplexing, after IF staining, H&E staining was performed at the last cycle.

Nuclease P1 stripping: Nuclease P1 has been examined as an alternative to DNase I. Following the imaging of PPI, the samples were incubated with nuclease P1 for 30 mins at 37° C. The samples were then washed with 20 mM EDTA three times, followed by 3 times 1×PBS wash.

H2 buffer: Add 30 ml of 5 M NaCl solution, 10 ml of 1 M Tris (pH 7.5), 0.943 ml of Triton X-100, 2.03 g of MgCl₂·6H₂O and 0.02% (wt/vol) NaN₃ to 960 ml of ddH₂O₃³⁵.

DMSO stripping solution: DMSO has also been tested to remove oligos and PLA signals. Following the imaging of PPI, the samples were incubated with Hybridization buffer (100 ml of DMSO with 400 ml of H2 buffer) for 1 min, followed by stripping buffer (62.5 ml of H2 buffer to 187.5 ml of DMSO) for 10 mins at RT. The samples were then washed three times with 1×PBS 35.

Imaging: A wide-field microscope, Keyence BZ-X800, was used for fluorescence and brightfield imaging. The fluorescent signals were detected by five filters with an excitation spectrum of Alexa Fluor 360 nm, 488 nm (FITC), 555 nm (TRITC), 590 nm (Texas Red), and 647 nm (Cy5). The exposure time was varied, but the exposure time for each marker across control and drug-treated HCC827 cells was consistent. The sample for cells grown on the coverslip was imaged at a 40× oil lens for 36 region-of-interest (ROI), and each ROI was imaged across 25-30 z-stacks with 0.4 m/stack. The whole slide tissue was imaged at a 20× dry lens, each ROI was imaged using autofocus and imaged at a 40× oil lens across 25-30 z-stacks with 0.4 m/stack. The resolution for the 20× dry lens and 40× oil lens was 0.37742 m/pixel and 0.18872 m/pixel respectively.

Image Processing: For 2D maximum projection images, stitched images provided by BZ-X800 Analyzer were used. The Hoechst channel from each cycle was used to register the images using a phase cross-correlation algorithm. On the other hand, for 3D per z-stack image processing, ROI images of 1024 by pixels were stitched by using a 30% overlap ratio using a code based on ASHLAR39. After stitching all cycle images were registered per z-stack as the microscope captured each ROI z-stack image at once.

Cell segmentation: Two distinct methods were used for single-cell segmentation for cell culture and tissue images. In cell culture images, Cellpose⁴⁰ deep learning algorithm was used whereas in tissue images Mesmer⁴¹ algorithm from Deepcell⁴² package was used for single-cell segmentation. Single cells in cell culture are more homogenous with more defined cell boundaries whereas cells in mouse tissue exhibited more variation in cell shapes and sizes. Cellpose was chosen for cell culture data and Mesmer for tissue data because the two algorithms were pre-trained on corresponding data modalities. Hoechst was used for nuclei segmentation and p-EGFR as a cytosolic marker.

PPI detection: PPI signals were detected using a custom algorithm leveraging a traditional image processing pipeline. More specifically, each PPI image was preprocessed as followed: images were first transformed using a top-hat filter of size 3 pixels to reduce the noise around PPI signals, then Laplacian of Gaussian images was used to detect bright local maxima as PPI signals. In 3D images, double detection of PPI was filtered out at the same position in consecutive Z stacks. After detecting all the PPI locations in 3D, PPI was observed within a 2.5-pixel radius (0.045 um) by creating a PPI neighborhood graph of radius threshold 2.5 pixels and getting all connected components in the graph. Finally, the mean position of the unique connected components in the graph was extracted as identified unique PPI signals in 3D.

PPI cell assignment in tissue samples: In the mouse tissue data, the cell membrane segmentations were not capturing the whole cell area. To avoid loss of PPI information due to cell segmentation error, a nearest-distance-based assignment method was used to assign the PPI signal detected and not in any cell segmentation mask. For each PPI signal not in any segmented cell regions, the nearest cell mask pixel was observed and if the distance was lower than a user-defined value, it was assigned to the cell to which the nearest cell mask pixel belonged.

Spatial Graph Construction: For each detected PPI in a cell, the corresponding 2D or 3D localization was extracted, and a node was assigned in the created graph. The node labels are assigned by creating a one-hot encoding of the corresponding PPI detected for the node. Delaunay triangulation was used to create edges connecting the nodes in the graph and therefore create a PPI spatial graph for each cell. The models are trained in a multi-instance learning framework, that is a cell label for each instance was assigned based on the cell treatment condition of a group.

SpPPI-GCN model: For the graph neural network, a 2-layer network was used consisting of graph convolutional layers of embedding size 16 for 5 PPI dataset 32 for 9 PPI dataset. The input of the model is the generated PPI graph for each cell with node feature represented by the PPI one-hot encoding. Each layer transforms the input as the following function: H^{l+1}=f(W^{l}H^{l}A*) with l the corresponding layer, f the activation function, H^{l} the node embedding matrix, W^{l} the weight matrix of the layer l, and A* the spectral normalized adjacency matrix. The spectral normalized adjacency matrix can be obtained with the following formula: A*=D^{-1/2}AD^{1/2} with A the corresponding adjacency matrix and D the degree matrix of A. Finally, the node embeddings are then aggregated by a pooling layer and two dense layers were then used to obtain prediction at the cell level (i.e., graph-level prediction).

Multi-instance learning baseline: A multi-layer perception baseline was used to compare the spPPIGCN network. In the same multi-instance learning framework, a class label from the cell treatment condition was assigned. The input of the model is the generated PPI graph for each cell with node feature represented by the PPI one-hot encoding. Here, stacked dense layers of embedding size 16 for 5 PPI dataset and 32 for 9 PPI dataset were used to obtain a node embedding. Each layer transforms the input as the following function: H^{l+1}=f (W^{l}H^{l}) with l the corresponding layer, f the activation function, H^{l} the node embedding matrix and H^{l} the weight matrix of the layer l. Finally, the node embeddings are then aggregated by a pooling layer and two dense layers were then used to obtain prediction at the cell level (i.e., graph-level prediction).

Multi-layer perception baseline: On the other hand, another multi-layer perception model was trained on the total PPI count per event for each cell using the same embedding dimension and number of layers. The input is the sum of each PPI class in (1) whole cell (5 and 9 input size for 5 PPI and 9 PPI dataset respectively) or divided by (2) cytosol and nuclei regions (10 and 18 input size for 5 PPI and 9PPI dataset respectively). The model output is the predicted treatment condition.

Machine learning baseline: Several machine learning models were used for a baseline on a total number of PPI events which includes Naïve Bayes, Random Forest, AdaBoost, Decision Tree, Support Vector Machine (SVM) and Gradient Boosting. The scikit-learn python library with default setting was used when training and testing these machine learning models. The total number of PPI events per whole cell was tested (1) and divided by subcellular regions (2) (Nuclei/Cytosol). Therefore, the input is the sum of each PPI class in (1) whole cell or divided by (2) cytosol and nuclei regions. The model output is the predicted treatment condition.

Prediction metrics: To compare the models' prediction abilities, a 5-fold cross-validation setting was used by separating the dataset into an 80% training set and a 20% validation set. Accuracy, ROC AUC score, and F1 score were used as metrics to evaluate the data prediction in the validation sets.

Statistical testing: The details of statistical tests employed in each case were provided in the figure captions. All P values were corrected for multiple testing and the statistical testing method was indicated in the figure captions. The following convention was used to indicate significance with asterisks: not significant (ns) (P>0.1), * (0.1>P>0.01), ** (0.01>P>0.001), *** (0.001>P>0.0001), and **** (P<0.0001).

### Example 2—Optimizing Single Cell Spatial Signaling Networks Using MEK/ERK or YAP Inhibitors in Combination with Osimertinib

Spatial signaling networks in single cells can validate previously established signaling alterations and provide new signaling coordination in EGFRm NSCLC cells (e.g., PC-9) under control, Osimertinib treatment only, MEK/ERK, or YAP inhibition only, and Osimertinib combined with MEK/ERK or YAP inhibition conditions. Signaling pathways mediate cell communication and cell functions such as cell proliferation, differentiation, and migration. Although the correlation between signaling markers' expression levels was extensively studied, signaling factors' spatial relation is still unclear. Also, the cell-to-cell heterogeneity in the signaling transduction could not be systematically profiled due to technical limitations.⁵⁵ Therefore, RapMIF can be utilized to resolve the subcellular spatial protein analysis in the MEK/ERK and YAP/Hippo signaling pathways (i.e., chosen because MEK/ERK and YAP use independent and dependent activation that can lead to secondary resistance) in established EGFRm PC-9 (responsive), PC-9/AR (first-line treatment resistance) and PC-9/GR/AR (second-line treatment resistance) human cell lines as key models to test drug treatments for EGFRm lung cancers. EGFR-TKI drug Osimertinib has been clinically tested. A portion of the patients suffers from resistance to the Osimertinib after first-line treatments. This resistance poses a challenge in clinical treatments of NSCLC patients with EGFR mutations. YAP in the Hippo pathway remains active under EGFR TKI and EGFR/MEK inhibitions, promoting the cells to enter a senescence-like dormant state in the absence of EGFR downstream signaling.¹⁰ Thus, recent combinatorial interventions using MEK/ERK⁸ or YAP¹⁰ inhibitors have demonstrated promising results to overcome the acquired resistance to Osimertinib. Thus, the establishment of spatial signaling states per cell in PC-9 and H1975 can translate the findings to combinatorial signaling therapies potentially that can later be used in translational settings.

Design and approach: RapMIF targets signaling protein markers (50-plex) including ERK-pERK, ERK1/2, pERK1/2, Mcl-1, Bim, p90RSK, and p-p90RSK to quantify the signaling crosstalk of ERK, AKT, mTOR, EGFR, and Wnt pathways. The previously established targets^{7-9,11,56} for assessing the efficacy of combination drugs (e.g., ACK1, c-Myc, ERK1/2, MEK1/2, YAP1, CD74, and AXL), Hippo signaling related upstream factors^{57,58} (MST1/2, LATS1/2, YAP/TAZ, 14-3-3, and MOB1), and YAP-related apoptosis targets¹⁰ (e.g., BMF, BCL2L11, SAT1, ERBB3, TNFSF10, and PDCD40) is used to reconstruct the spatial signaling network. These targets are benchmarked by mass spectrometry and RNA sequencing results in NSCLC culture and in vivo models, allowing to define on a “targeted panel” from the transcriptome or proteome-wide datasets in Osimertinib therapy. The number of cells needed to accurately reconstruct the spatial signaling network can be determined. The accuracy of the network depends on the estimated pairwise distances of clusters, which is defined as the percentage of different clusters within the neighborhood of a given cluster. It is estimated that with 1000 cells (TABLE 4), the standard deviation of the distance estimates will be within 5% of the true value. Thus, 1000 cells, can accurately construct the network.

TABLE 4
Data structure of RapMIF. Identify Pixel phenotype (UMAP, clusters) and Network
(Pixel's neighborhood). M1-50: Markers.

| Conditions | Pixels | M1 | M2 | M3 | . . . | M50 | Cluster # | Network |
|---|---|---|---|---|---|---|---|---|
| PC9 cell1 | Pxl (x1, y1) | 5 | 15 | 20 | . . . | 8 | cluster 1 | node 1-5 |
| Control | Pxl 2 (x2, y2) | 3 | 7 | 10 | . . . | 20 | cluster 5 | node 1-5-3 |
| | Pxl 3 (x3, y3) | 8 | 9 | 12 | . . . | 15 | cluster 3 | node 3-5 |
| | . . . | . . . | . . . | . . . | . . . | . . . | . . . | |
| | Pxl 100 (x100, y100) | 6 | 5 | 13 | . . . | 13 | cluster 11 | node 11 |
| | . . . | . . . | . . . | . . . | . . . | | | |
| PC9 cell1000 | Pxl (x1, y1) | 3 | 6 | 6 | . . . | 6 | cluster 2 | node 2-3 |
| Control | Pxl 2 (x2, y2) | 2 | 6 | 11 | . . . | 11 | cluster 3 | node 2-3-5 |
| | Pxl 3 (x3, y3) | 7 | 4 | 15 | . . . | 15 | cluster 5 | node 5-3 |
| | . . . | . . . | . . . | . . . | . . . | | | |
| | Pxl 150 (x150, y150) | 6 | 5 | 13 | . . . | 13 | cluster 13 | node 13 |
| | . . . | . . . | . . . | . . . | . . . | | | |
| PC9 treated | Pxl (x1, y1) | 2 | 4 | 6 | . . . | 6 | cluster 2 | node 2-4 |
| cell1 | Pxl 2 (x2, y2) | 2 | 3 | 11 | . . . | 11 | cluster 4 | node 2-4-7 |
| concentration | Pxl 3 (x3, y3) | 12 | 4 | 15 | . . . | 15 | cluster 7 | node 4-7 |
| combination | . . . | . . . | . . . | . . . | . . . | . . . | . . . | |
| | Pxl 120 (x120, y120) | 5 | 8 | 11 | . . . | 11 | cluster 12 | node 12 |
| | . . . | . . . | . . . | . . . | . . . | | | |
| PC9 treated | Pxl (x1, y1) | 3 | 6 | 6 | . . . | 6 | cluster 6 | node 6-1 |
| cell1000 | Pxl 2 (x2, y2) | 2 | 6 | 11 | . . . | 11 | cluster 1 | node 6-1-9 |
| concentration | Pxl 3 (x3, y3) | 7 | 4 | 15 | . . . | 15 | cluster 9 | node 1-9 |
| combination | . . . | . . . | . . . | . . . | . . . | | | |
| | Pxl 140 (x140, y140) | 6 | 5 | 13 | . . . | 13 | cluster 15 | node 15 |

Signaling networks depend on the quality of detection antibodies and inhibitor efficacy in the image-based IF staining experiments. Thus, genetic perturbations⁷ can be used to validate one antibody and one inhibition target at a time before multiplexing experiments. Specifically, a signaling protein of interest (e.g., ACK1, c-Myc, ERK1/2, MEK1/2, YAP1, CD74, BMF, and AXL) can be targeted using small hairpin RNA (shRNA) or small interfering RNA (siRNA) to perform gene knockdown, providing robust suppression of the gene expression and reduction in the protein production levels. A sequence-specific shRNA (Millipore Sigma) for the gene of interest can be performed in pLKO¹. lentiviral vector to knock down the gene target, providing the genetically perturbed PC-9 validation for the RapMIF. Similarly, a siRNA (Santa Cruz Biotechnology) for a gene of interest can be transfected into the PC-9 cell lines in 6-well plates using the HiPerFect transfection reagent (QIAGEN; Germantown, MD). After 48 h, the cells were collected and re-plated in 24-well plates for RapMIF experimental validations. Non-silencing scramble control siRNA (siCtrl) can be performed as before⁵⁹.

Established human cell lines EGFRm PC-9 (responsive), PC-9/AR (first-line treatment resistance), and PC-9/GR/AR (second-line treatment resistance) serve as useful models to test drug treatments for EGFR mutant lung cancers. EGFR-TKI drug Osimertinib has been clinically tested. A portion of the patients suffers from resistance to the Osimertinib after first-line treatments. This resistance poses a challenge in clinical treatments of NSCLC patients with EGFR mutations. Thus, the establishment of signaling states per cell line translates the findings to clinical applications. It has been shown that PC-9 cell cultures can be used to study synergistic combinations of Osimertinib with ERK (VRT752271), MEK (Trametinib), or YAP/TEAD (MYF-01-37) inhibitors^{7,8,10}. Cell apoptosis is enhanced by Osimertinib combined with VRT, Tram, and YAP/TEAD inhibitor drugs. Cell viability counts are performed by sulforhodamine B (SRB). Control experiments can be performed by cells growing in DMSO. Apoptosis can be measured by caspase/PARP cleavage using western blot (WB), and Annexin-V can be quantified by flow cytometry. Drug treatments can be performed in the 0-10 nM for Osim, 0-100 nM for Tram, 0-1000 nM for VRT, and 0-1000 nM for MYF-01-37 in PC-9 cell lines. Thus, RapMIF experiments are designed for 50-plex markers in this range to profile signaling networks in PC-9, PC-9/AR, and PC-9/GR/AR cells for a combination of these four drugs, yielding strategies to overcome the acquired resistance to Osimertinib. Given the marker expressions for each pixel from all cells, a regression-based analysis is run for all markers using drug concentrations and drug combinations as covariates (TABLE 4). This analysis provides markers that are associated with drug treatment.

RapMIF can perform 50-plex signaling measurements in PC-9, PC-9/AR, and PC-9/GR/AR cells using 24-combinatorial drug treatments with four different concentrations, each in a glass-bottom 96-well format during 0.5-2 days cell cultures. Quantification of cell survival is performed by SRB assay. Immunostaining of Annexin-V and Caspase 3 provides cells committing to apoptosis. In this analysis, the data from all the conditions is pooled together and clustered at once. This process reveals signaling networks on a reference map using heatmaps, t-distributed Stochastic Neighbor Embedding (t-SNE), and Uniform Manifold Approximation and Projection (UMAP) representations. Spatial cross-correlation can determine the spatial similarity of multiple cell groups on the colored cluster images. RapMIF is then provide subcellular and cell-to-cell heterogeneity of these synergistic combinations to shed light on treatment for NSCLCs with EGFRm.

Experimental results: A panel of 8 cycles consisting of 20-markers that includes p-EGFR, Phalloidin, WGA, p-AKT, p-β-catenin, RNF 43, non-p-β-catenin, APC, DKK1, Cyclin E, EMMPRIN, Wnt1, AKT, p-mTOR, mTOR, DKK2, AXIN1, EGFR, Concanavalin A, and Cyclin D1 was designed for RapMIF experiments in EGFRm PC-9 cells. To reduce sample incubation time in indirect IF, the antibodies were conjugated with one of three Alexa dyes using Lightning-Link Rapid Alexa Fluor Antibody Labeling Kit (Novus). The order of markers in the experiment was decided based on the marker's brightness and expression level. After mounting, permeabilizing, and blocking the cells on a coverslip, the coverslip was ready for cyclic experiments. In the eight-cycle, the sample with antibodies was incubated for 30 minutes or 1 hour. Following imaging, the dyes were bleached in the NaOH—H₂O₂ NaBH4, and LiBH4 cocktails, and other fluorophore inactivation methods and antibody stripping methods were tested to make rapid switching per cycle. Even though the base-hydrogen peroxide mixture bleaches Alex 488, 555, and 647 at different rates, 30-45 min of treatment is sufficient. In the experiment, cell loss was not observed in any of the cycles. Following fluorophore inactivation, the coverslip was re-blocked with cell staining media, and the next process was performed.

An eight-cycle RapMIF experiment yielded 18-channel images of the samples at the single-cell level. The multiplex single-cell measurement reveals the interactions of signaling proteins in the subcellular networks. Pixel-level correlations of signaling protein expressions in the concatenated RapMIF data (see TABLE 4) provided co-regulation on a heatmap using z-scored spatially resolved data analysis. Note that pixel-level analysis is used to compute local protein density in each cell.

The RapMIF experiment was then performed in EGFRm NSCLC sensitive cell line, PC-9 cells. Osimertinib has shown a prominent effect in inhibiting PC-9 cells, and the cell survival rate has decreased as the concentration has increased from 0 to 10 nM under a 3-day treatment^{7,8}. PC-9 cells responding to Osimertinib treatment have been shown to develop acquired resistance. A preliminary study investigated whether p-EGFR (Tyr1086) would be inhibited by Osimertinib in PC-9 cells. Osimertinib would then affect the proliferation and growth of the PC-9 cells. Under the incubation of 48-hour drug treatment, the number of cells adhering to the coverslips has decreased as the drug concentration has increased. Following drug treatments, the RapMIF data provided the imaging maps of up to 25-plex signaling proteins in PC-9 with (n=451 cells) and without (n=531 cells) the treatment of 40 nM Osimertinib. Validation experiments decreased the p-EGFR expression as the drug concentration increased from 0 to 20 nM to 40 nM, whereas p-EGFR exhibited relatively higher expression under 60 nM than 40 nM. Thus, 40 nM Osimertinib was chosen for RapMIF multiplexing. The Osimertinib treatment increased the WNT1 signaling, while the p-EGFR (Tyr1086) alterations remained minimally enhanced in the heatmap of combined clustering from the signaling factor maps of untreated and 40 nM Osimertinib PC-9 samples. The heatmap indicated the single-cell alterations of signaling in response to Osimertinib treatment, providing sub-groups of control (blue) and drug-treated (red) in the normalized heatmap (dark blue to light orange). The results validated that signaling intensities concurrently alter in Osimertinib drug treatments. After showing similarities of intensities in the multiplexed data at the single-cell level, the pixel-level co-regulation of signaling subsets was determined using clustering analysis in the next analysis.

Spatial signaling factor expression levels in the pixel-level RapMIF datasets were concatenated together with untreated (control) and drug-treated cells, allowing the comparisons of pixel clusters (identified by UMAP) containing a subset of signaling networks modules across different conditions of PC-9 experiments. An interesting finding was that the spatial signaling network maps of the PC-9 cells after 40 nM Osimertinib drug treatment exhibited more dispersed (i.e., less aggregated) distribution in the cytosol, noted as clusters 0, 1, and 2, compared to the control sample. Subcellular spatial networks (identified by pixel neighbors of clusters) were represented as a physical neighborhood map for the assessment of protein-protein proximity at the pixel level for RapMIF data from the control (untreated) and 40 nM Osimertinib treated PC-9 cells. Each node corresponds to one of the cluster phenotypes of pixels (see TABLE 4, Cluster number #column) in the form of cluster number. The color of the node matched with the cluster color assigned to the pixel's identity. The length of the connected line represented the pixel's spatial neighborhood in the form of cluster groups e.g, 1, 5, 3 (see TABLE 4, Network column). The size of the node showed the percentage of the cluster out of all clusters. The results indicated how signaling subsets co-localize or co-regulate in the same or neighbor pixels.

### Example 3—Optimizing Single-Cell Radiometric Signaling Interaction Method in EGFRm Cell Lines Using MEK/ERK or YAP Inhibitors in Combination with Osimertinib

A study was conducted with the following goals:

1) Compare subcellular image-based protein interaction assays using multiplexed proximity ligation assay in sensitive and resistant EGFRm NSCLC lines (HCC827 vs HCC827/AR and PC9 vs PC-9/AR and PC9/GR/AR) using control, Osimertinib only, a MEK/ERK or YAP inhibitor only, and their combinations.

2) Model alterations in cellular protein interaction maps built from distinct subcellular locations (endoplasmic reticulum, mitochondria, nucleus, and cytosol) of sensitive and resistant EGFRm NSCLC lines.

Spatial signaling interactome in single cells can validate previously established signaling alterations and provide new signaling coordination, and combination drug-specific ratiometric protein interactors in sensitive and resistant EGFRm NSCLC lines (HCC827 vs HCC827/AR and PC9 vs PC-9/AR and PC9/GR/AR) under control, Osimertinib treatment only, MEK/ERK or YAP inhibition only, and Osimertinib combined with MEK/ERK or YAP inhibition conditions. PPI assays have shown the efficacy of Osimertinib treatments on blocking the specific EGFR downstream signaling (e.g., Grb2) using a single-molecule-based pulldown assay⁶⁰. This method generated in vitro fluorescence images when PPI was achieved within two pairs of proteins through antibody-based detection and isolation procedures of cell extracts, causing the loss of spatial features in cell shapes, morphology, and signaling. While single-molecule PPI provides alterations of signaling in cell extracts, protein interaction pairs necessitate in-situ labeling and imaging technology to build a direct PPI in intact cells and tissues.

Besides, recent pieces of evidences⁶¹ have suggested the role of EGFR's tyrosine kinase protein interaction partners on the subcellular localization of PPIs across 13 distinct regions of signaling interactions identified by a guilt-by-association pipeline in NSCLCs⁶¹. The correct predictions (green) of subcellular protein positions were limited to less than half of the experimental and prediction datasets, demonstrating a challenge to accurately reconstruct PPIs' subcellular distributions. Thus, the ultimate validation is lacking to co-localize subcellular compartments and PPIs in the multiplexed single-cell data.

Design and approach: Identification and validation of multiplexed signaling interactor targets. Single-cell spatial interaction of PPIs can be targeted for EGFR-based network activity (n+) that contains EGFR, CDH1, P IK3R3, DOK5, TNC, PRKD1, VAV3, PARK7, ANKS1B, ALB, EGF, RB14, SLFN13, AP2B1; EGFR-related resistance markers including FAM157A, SCYL2, ZNF503, AP2B1, TMSBX4, KRT7, FOXA1, CDKN2AIPNL, ERBB3, AP2A2, RBM10, PEBP1, NME2, PLEC, KRT18, and TMSB10, all identified in the EGFR network oncogenesis results⁶¹ Additional targets from the previous Osimertinib drug-induced protein alterations^{7,8} can also be included in this interaction panel.

RapMPI can then profile 25-protein pairs in sensitive and resistant EGFRm NSCLC lines (HCC827 vs HCC827/AR and PC-9 vs PC-9/AR and PC-9/GR/AR) using control, Osimertinib only, a MEK/ERK or YAP inhibitor only, and their combinations. This study can include 5 cell types, 5 drug combinations, and 4 concentrations that can be performed in a 96-well and cultured for 12-24 hours, followed by fixation and RapMPI analysis of protein pairs. In a small pilot experiment, the RapMPI can validate the known and previously uncovered PPIs among proteins of the network1 (WNT1, DKK1, DKK2, RNF43, APC, AXI N1, EGFR, AKT, p-AKT, mTOR, p-mTOR, β-Catenin, Cyclin D1, Cyclin E, and EMMPRIN) and network 2 (YAP/TAZ, pYAP/TAZ, ERK, MEK, RAF, EGFR, RAS, RASSF, MERLIN, NF2, MOB1, MST1/2, and LATS1/2) targets in the same single cells of each cell culture and condition within this 96-well plate. This pilot PPI module and similar experiments can decipher pairwise PPI ratios (e.g., PPI1/PPI2 in the same/different pixel pairs) in many other PPI modules as a function of microwell descriptions. Ratios are calculated to model stochiometric protein balance^{62,63} in the signaling interactome. A multivariate binomial generalized linear model can be run to study the association of ratiometric interactions and covariates including different cell types, drug combinations, and concentrations (TABLE 5). The result can identify ratios that are associated with the covariates.

TABLE 5
Data structure of RapMPI. Identify Interaction ratios (e.g., I1-2/I3-4) and Organelle.
I1-2 and I3-4 etc.: Interaction pairs.

| Conditions | Pixels | I1-2 | I3-4 | I5-6 | . . . | I49-50 | Ratios | Organelle |
|---|---|---|---|---|---|---|---|---|
| PC9 cell1 | Pxl (x1, y1) | 5 | 0 | 0 | . . . | 10 | I1-2/ | ER |
| Control | | | | | | | I49-50 | |
| | Pxl 2 (x2, y2) | 0 | 4 | 8 | . . . | 0 | I3-4/I5-6 | Mito |
| | Pxl 3 (x3, y3) | 0 | 0 | 6 | . . . | 12 | I5-6/ | Golgi |
| | | | | | | | I49-50 | |
| | . . . | . . . | . . . | . . . | . . . | . . . | . . . | |
| | Pxl 100 (x100, y100) | 3 | 9 | 0 | . . . | 0 | I1-2/I3-4 | ER |
| | . . . | . . . | . . . | . . . | . . . | | | |
| PC9 cell1000 | Pxl (x1, y1) | 0 | 0 | 7 | . . . | 14 | I5-6/ | Mito |
| | | | | | | | I49-50 | |
| Control | Pxl 2 (x2, y2) | 8 | 4 | 0 | . . . | 0 | I1-2/I3-4 | Golgi |
| | Pxl 3 (x3, y3) | 5 | 0 | 15 | . . . | 0 | I1-2/I5-6 | ER |
| | . . . | . . . | . . . | . . . | . . . | | | |
| | Pxl 150 (x150, y150) | 3 | 0 | 0 | . . . | 11 | I1-2/ | Mito |
| | | | | | | | I49-50 | |
| | . . . | . . . | . . . | . . . | . . . | | | |
| PC9 treated | Pxl (x1, y1) | 3 | 0 | 0 | . . . | 8 | I1-2/ | ER |
| cell1 | | | | | | | I49-50 | |
| concentration | Pxl 2 (x2, y2) | 0 | 7 | 9 | . . . | 0 | I3-4/I5-6 | Mito |
| combination | Pxl 3 (x3, y3) | 0 | 0 | 3 | . . . | 5 | I5-6/ | Golgi |
| | | | | | | | I49-50 | |
| | . . . | . . . | . . . | . . . | . . . | . . . | . . . | |
| | Pxl 120 (x120, y120) | 4 | 8 | 0 | . . . | 0 | I1-2/I3-4 | ER |
| | . . . | . . . | . . . | . . . | . . . | | | |
| PC9 treated | Pxl (x1, y1) | 0 | 0 | 10 | . . . | 7 | I5-6/ | ER |
| | | | | | | | I49-50 | |
| cell1000 | Pxl 2 (x2, y2) | 4 | 3 | 0 | . . . | 0 | I1-2/I3-4 | Mito |
| concentration | Pxl 3 (x3, y3) | 4 | 0 | 11 | . . . | 0 | I1-2/I3-4 | Golgi |
| combination | . . . | . . . | . . . | . . . | . . . | | | |
| | Pxl 140 (x140, y140) | 2 | 0 | 0 | . . . | 6 | I1-2/ | ER |
| | | | | | | | I49-50 | |

The reconstructed RapMPI data can then associate the alterations in subcellular protein interaction maps to the distinct organelles (e.g., Golgi: Sortilin & GOLPH4, Endoplasmic reticulum: ATF6, Mitochondria: TOM20, nucleus: DAPI, and cytosol: Actin, β-Tubulin) of sensitive and resistant EGFRm NSCLC lines. The RapMPI experiment can be followed by an organelle identifier RapMIF experiment using these established markers, yielding and validating the subcellular localizations of PPIs (see TABLE 5 last column) in individual cells rather than prediction pipelines previously reported and widely used in cancer signaling for evaluating the drug response and resistance.

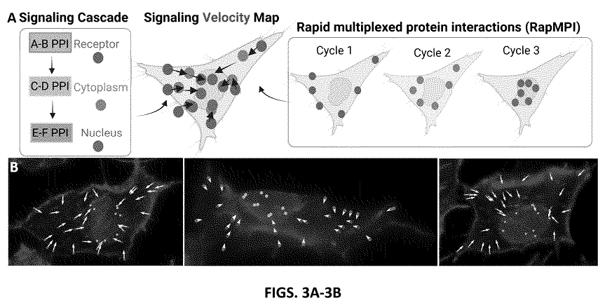

Spatial maps of subcellular PPIs and subcellular localization patterns can then be modeled as spatial signaling velocity maps, akin to the RNA velocity concept⁴⁵. RapMPI results provide multiplexed PPIs in receptor, cytosol, and nuclear locations. Computational modeling can include position (x-y-z) and signaling interaction (e.g., A-B, C-D, and E-F PPIs). Using the priori information on signaling cascades obtained from the bulk and imaging assays, an arrow strategy can be used to trace signaling information flow in each lung cancer cell. For instance, a simple scenario depicts the probabilistic signaling interactions denoted by arrow fields within a cell using the signaling cascade across A-B→C-D→E-F, wherein each PPI was colored by magenta, green, and blue, respectively (FIG. 3A). Experimental and simulated validations (FIG. 3B) can be performed to establish signaling velocity as a new data visualization and bioinformatics method for quantifying spatial interactors and their subcellular distributions.

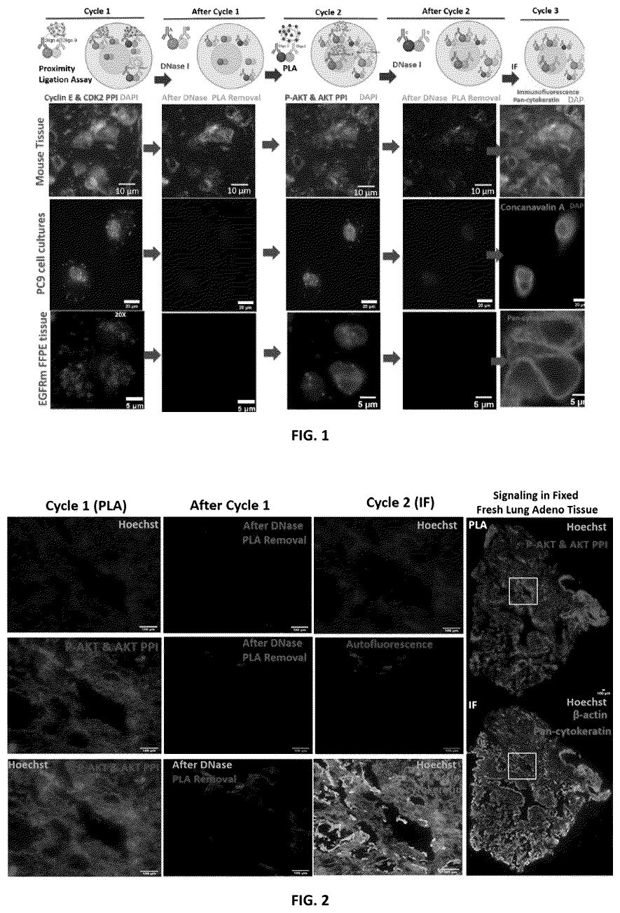

Experimental results: The proof-of-principle of RapMPI was performed by using the two sets of protein targets using one set of distinct antibodies (Cyclin E & CDK2, PPI1) and another total and phosphorylated site of Akt (PPI2), providing unique PPI within the same cell during phosphorylation and Cyclin E and CDK2 interactions within proximity. For instance, the interaction of Cyclin E and CDK2 controls cell cycle regulation, and, at the same time, the interaction between p-AKT(Ser473) and AKT indicates the phosphorylation and activation of p-AKT by PI3K. Therefore, the interaction between p-AKT (Ser473) and AKT indicates the full activation of AKT. To probe the PPI1 of Cyclin E and CDK2, two sets of PLA assemblies were performed in the fresh PDX lung tumors, PC9 cultures, and EGFRm FFPE human tissues, yielding ubiquitous PPI in every single cell denoted by red dots (i.e., the same color for two distinct PPI validated multiplexing). After DNAse I treatment, the PLA was removed. A second cycle was then performed by a different protein pair of Akt and p-Akt (PPI2) to detect previously unidentified PPIs. The PLA in 2 cycles provided PPIs in different subcellular locations (FIG. 1). In the 3^{rd} cycle, regular IF detected pan-cytokeratin stained (CK+) tumors. In a separate experiment, the same PLA for Akt and p-Akt was performed in fixed fresh lung tumors, yielding cellular PPI distributions (FIG. 2).

The PLA removal was also effective and the subsequent cycle was then used to detect immunofluorescence of pan-cytokeratin and B-actin markers, indicating single-cell and tumors (CK+). This demonstration sets the practicability of multiplexing PLAs in tissues. Thus, RapMPI is feasible both in cultures and tissues.

Discussion: Spatial interaction ratios of PPIs from single-cells (RapMPI) and their subcellular localization on distinct organelles (RapMIF) is projected on high-dimensional visualization maps (tSNE & UMAP) to define necessary and sufficient “resistance” or “sensitivity” to drug combinations for rationally designed NSCLC therapies. The signaling velocity concept can be an emerging approach to quantify signaling responses by tracing PPIs across the subcellular volumes quantitatively. The proposed signaling framework is widely applicable to many other TKI proteins and their spatial interactome analysis.

To overcome any issues of antibody already bound, which would lead to the second staining suffering from the previous antibody blocking the e.g., A antigens, two strategies can be used to overcome this issue. The first one is to strip the antibody instead of PLA removal, making the antigens from the previous cycles accessible. The second one is to access distinct phosphorylation sites of the same antibody to make the relevance of tri-protein interactions. These strategies open up directions in scaling up the barcoding space of RapMPI assay from F×N (linear) to F^{N} (exponential), where F is the number of colors and N is the number of cycles. This further advances the capacity of spatial PPI mapping in single-cells to the proteome level as next-generation spatial proteomics technology.

### Example 4—Validating Signaling Networks and Interaction Assays In Vivo Using MEK/ERK or YAP Inhibitors in Combination with Osimertinib in Mouse Xenograft and PDX Models and Human Tissues

A study was conducted with the following goals:

1) Construct single cell and subcellular networks and protein interactions in tissues from Osimertinib-treated PC-9 xenografts or EGFRm PDX models that are sensitive to EGFR-TKIs including pretreatment or baseline condition (n=5), 1-week post-treatment (n=5), and after 1-month treatment (n=5) or until the resistance occurs using 10 tissue slices per condition.

2) Develop a predictive signaling response tool using in vivo signaling networks and interaction ratios from human tissues (n=20) obtained from EGFRm patients collected as the baseline pretreatment condition.

Single cell and subcellular networks and protein interactions in tissues extracted from Osimertinib-treated PC-9 xenografts or an EGFRm PDX model and EGFRm human biopsies can confirm or reform signaling co-regulation and interactors from in-vitro cell cultures in physiologically relevant in-vivo models and translational settings. While cell cultures shed light on signaling mechanisms to evaluate the acquired resistance to Osimertinib, molecular heterogeneity of in vivo tumors further contributes to the differences in the resistance mechanisms. Osimertinib-treated PC-9 xenografts or EGFRm PDX models provide signaling response studies in both pre and post-treatment conditions, while the human studies are only limited to pre-treatment biospecimens. Thus, this study can employ RapMIF and RapMPI to profile tissues from PC-9 xenografts or an EGFRm PDX model that is sensitive to EGFR-TKIs including pretreatment or baseline condition, 1-week post-treatment, and after 1-month treatment or until the resistance occurs.

EGFR-TKI inhibitors' role in improving NSCLC patients' treatment outcomes has been identified in phase 3 clinical trial. Osimertinib (80 mg once daily) showed longer progression-free survival and overall survival than other EGFR-TKI drugs, while the tolerability was improved compared to that of others³. However, a fraction of patients show resistance to first-line therapy, and some others also exhibit secondary resistance in other signaling and genetic mechanisms.4 Previous work on immunohistochemistry (IHC) and plasma analysis of these patients with EGFR mutations yielded significant variability between the patients and lung cancer subtypes^{64,65}. The selection of patients for Osimertinib can benefit from personalized molecular profiling of patient tissues for clinical applications. Thus, RapMIF and RapMPI profile spatial signaling networks and interactions in baseline pre-treatment patient biopsies with EGFR mutations as a molecular screening method as a potential companion diagnostic tool using multivariate classification.

Design and approach: RapMIF and RapMPI can be performed to reconstruct single cell and subcellular networks and protein interactions in tissues from Osimertinib-treated PC-9 xenografts or an EGFRm PDX model that is sensitive to EGFR-TKIs including pretreatment or baseline condition (n=5), 1-week post-treatment (n=5), and after 1-month treatment (n=5) or until the resistance occurs using 10 tissue slices per condition (n=5 tumors). Previously validated⁸ drug concentrations can include Osim (15 mg/kg, once per day), Tram (1 mg/kg, once/2 days), or the combination of Osim and Tram. The YAP inhibitor (MYF-01-37) can be optimized in the range of 1-15 mg/kg once/1-3 days for establishing the efficient pharmacologic inhibition of Osim, Tram, and MYF. Tumor size (0.5 length×width²) can be measured with caliper quantities. Fresh tissues (10 slices per condition) can be extracted and stored at −80° C., followed by 50-plex RapMIF and RapMPI experiments. Spatial image-based data can be processed by joint bio statistical modeling for comparing distinct conditions. It is estimated that with 1000 cells, the standard deviation of the distance estimates will be within 5% of the true value. Thus, with 1000 cells, the network can be accurately constructed.

Signaling networks in tissues of EGFR mutant patients in response to EGFR TKIs treatments would help predict the drug response and isolate signaling mechanisms responsible for drug resistance. RapMIF & RapMPI can image and visualize 50-plex signaling biomarkers to predict development, progression, and response to drug treatment of lung cancers in retrospective studies involving pre-treatment biopsies and Osimertinib treatment outcomes. The study focuses on baseline because practically obtaining post-treatment biopsies are limited to small biopsies used for clinical purposes and not sufficient for research investigations. Initially, RapMIF & RapMPI can quantify a 50-plex antibody panel in 27 tissue cores from lung cancer patients with EGFR mutations and another wild-type group (TriStar Tech Group, SKU: 69572826-2826). The feasibility of tissue imaging can be tested, and the results can be compared to cell colonies obtained in cancer cultures. This comparison can be performed by clustering all the data into one pool and comparative analysis of signaling expression levels and subcellular patterns. Next, clinically archived tissues (n=20 patients, 5 tissue sections each) from EGFRm NSCLC patients can be used for signaling network validations. These retrospective tissues can be from two patient groups comprising Osimertinib resistant (n=10) and responder (n=10) cohort, providing opportunities to correlate signaling networks and clinical outcome/survival. RapMIF & RapMPI can then profile FFPE & fresh frozen tissue sections after fixing and performing to validate tissue staining and analysis protocols (FIG. 2). The resulting spatial signaling networks and interactors can be integrated into a single data analytics pipeline, providing multivariate classification^{67,68} of spatial signaling features per patient using random forest and naive Bayes classifiers. The EGFRm data size of n=10 responder and resistant and 10 tissue sections each with n=1,000 single cells and diverse signaling data can provide decent multivariate classification (0.95 power) using a permutation test, nearest neighbor, or a Gaussian model.

Experimental results: Continuous and intermittent pharmacologic inhibition of MEK/ERK signaling was demonstrated to delay the emergence of Osimertinib resistance in PC-9 xenografts in nude mice. Complementary study¹⁰ showed Osimertinib and trametinib treatment of mice bearing xenograft tumors from YAP1 knock-out (KO) PC-9 or, from the corresponding control cells, led to a durable response for the entire 4-week treatment period.

RapMIF screened up to 25-plex signaling proteins in a tissue lung adenocarcinoma microarray (BS04081a, Biomax) that contained 63 cores, 21 patient cases, varying from normal, stage I, II, III tumor. The tissue cores were then classified into pan-cytokeratin positive and negative regions. Patients with higher tumor stages demonstrate upregulated WNT and AKT pathways with larger variation in the pan-cytokeratin-positive region. The p-β-catenin was upregulated in tumor regions while the constituent activation of WNT and AKT pathway could promote the stabilization of non-p-β-catenin and the degradation of p-β-catenin.⁶⁶ Multiplexed signaling maps across 55 cores were classified by stages and pan-cytokeratin expression at the single-cell level. The signaling markers in pan-cytokeratin-positive regions exhibited higher expression levels than normal tissue regions. The majority of signaling markers, RNF43, EGFR, H3K27me3, WNT1, p-EGFR, AKT, mTOR, and cyclin D1, demonstrated upregulation in the tumor regions.

Discussion: Non-small cell lung cancer (NSCLC) comprises 80% to 85% of lung cancer, and it is the second leading cause of death after cardiovascular disease.¹ While there is an increasing number of treatments for NSCLC, the survival rates are slightly increasing from 15% to 20%.² NSCLC patients with epidermal growth factor receptor (EGFR) gene mutations, harboring 19 deletions (19del) and exon 21 point mutation (L858R), are treated with EGFR tyrosine kinase inhibitors (EGFR-TKIs). One of these drugs has been Osimertinib that extended patients' progression-free survival and overall survival in phase 3 clinical trial settings.³ However, a subset of patients have primary resistance to these therapies and the initial responders will ultimately develop secondary or acquired resistance to Osimertinib.⁴ Tumors' molecular and cellular alterations such as the appearance of new EGFR resistant mutations and signaling rewiring cause emergence of acquired resistance and eventual cancer progression. Bulk genomics and proteomics assays have yielded significant signaling mechanisms in cells and tissues. However, these signaling assays fail to capture single-cell and subcellular features in the spatial context in cancer specimens. They also lack cell-to-cell variations due to the averaging of samples during the measurements. Thus, there is an essential need for single-cell imaging and analysis methods to visualize individual cells' signaling dynamics and protein interactions of intracellular labels under drug perturbations.

This proposed system directly visualizes signaling processes in single cells as signaling factors interact with each other in fixed samples. Image-based data spatially resolve cell membrane, cytosol, organelles, and nucleus to reconstruct direct signaling maps in EGFR mutant (EGFRm) cell models. EGFRm cell lines such as PC-9 are sensitive to Osimertinib and the expected signaling distributions were obtained from western blot data, providing opportunities to validate the proposed signaling visualization technology. In-vivo validations of signaling responses can be obtained from PC-9 engrafted mouse models and human tissues from EGFRm NSCLC patients. The proposed platform can inform signaling therapy design or Osimertinib resistance at ultimate resolution and sensitivity.

Multiplexed subcellular protein imaging methods are desirable for spatial signaling networks as a screening platform for clinical use. Provided is an advanced multiplexed protein imaging method to provide rapid experiments, automated microfluidics-based re-labeling and removal of signal, and imaging on the same platform. Here, a rapid multiplexed immunofluorescence (RapMIF) method can be developed to visualize many molecules (30 to potentially 100 parameters) at high optical resolution in cells and tissues. The multiplexing three-color experiments label three antibodies for unique targets at a time. In this experiment, “n cycles” can provide “n×3” markers in the reconstructed image. For instance, ten cycles can provide 30 biomarkers per cell. The experiment is performed on a wide-field microscope using 40× to 60× optical magnification to capture subcellular details. Fixed (1.6% formaldehyde) and permeabilized (0.5% Triton-PBS1×) cells are then labeled, washed, and re-labeled (after signal removal by rapid chemistry⁴⁰) using an autosampler, valve, and fluidic components. The sample is positioned on a custom microscope stage that allows both sample treatments and imaging on the same RapMIF platform.

Thirty signaling protein factors are defined based on prior literature on signaling cascades. Both total and phosphorylated proteins are targeted in the RapMIF experiments for mapping spatial distributions of signaling patterns across the cytosol and nucleus of a cell. RapMIF maps thousands of cells to target thirty to hundreds of markers in 24 hours over three dimensions after integrating automated labeling, imaging, and analysis pipelines. Spatial bioinformatics models were also developed to create a digital spatial signaling network model of cells as a result of pixel-level clustering of signaling factor distributions measured at a high-optical resolution up to super-resolution⁴¹ maps.

Deciphering the PPIs within Osimertinib-related signaling pathways reveals the potential targets for overcoming the acquired resistance. Proximity ligation assay (PLA), also referred to as Duolink PLA technology,^{42,43} provides an approach to detect direct PPIs in situ at endogenous protein levels. For the detection of one PPI, the cells need to be stained with two primary antibodies targeting two different proteins or proteins phosphorylated at different sites. These primary antibody pairs are conjugated to PLA probes with one PLU, and another one to MINUS. The PLA probe, attached to the heavy chain of the primary antibody, contains a unique oligonucleotide. When the protein of interest interacts with each other, the DNA probes from two antibodies hybridize and ligate to form circular DNA. The amplified circular DNA can be visualized using fluorescence microscopy. The PLA detection kit allows visualization and quantification of the individual PPI. Multiplexing PLA is limited to 3 protein pairs. Here, a highly multiplexed PLA assay, termed rapid multiplexed protein interactions (RapMPI), can be developed based on sequential PLA labeling of 3 antibody pairs, removal of PLA assembly using DNase I, and re-labeling of the same cell with a different set of 3 antibody pairs. This process is then repeated “n” times, to create a multiplexed protein interaction map in the same single cell. For instance, 10 cycles would provide 30 protein pairs identified by 60 antibodies specific to protein pairs of interest. RapMPI is akin to sequential FISH (our previous invention to multiplex RNAs),⁴⁴ but it has a unique DNA assembly to detect protein-protein proximity within 20-nm physical range. In the RapMPI assay, the proteins are targeted by the same antibodies used for RapMIF, however, antibodies are conjugated to plus and minus oligos for building a rolling circle amplification (RCA) to make individual protein interactions detectable as a diffraction-limited spot in the microscopic image of a cell.

Compared to other PPI assays using overexpression of vector engineered cells,³⁹ the native state of protein interactions are detected by the RapMPI assay, an important feature of PPIs in physiologically relevant conditions. The PLA assay was assembled using the commercial DuoLink platform, but RapMPI utilizes Co-detection by Indexing (CODEX) protocols to develop custom PLA panels for highly multiplexed subcellular protein interactions maps. Automated microfluidics and spatial bioinformatics analysis are shared between the RapMPI and RapMIF platforms using the same automated valve, fluidic, and autosampler setup mounted on the sample holder located on the microscope. One key difference is to spatially trace subcellular signaling cascades using prior information on downstream signaling molecules. This allows the creation of “signaling velocity” maps akin to RNA velocity⁴⁵ implemented by the differential equations of biomolecular conversions.

The proposed multiplexed in-situ signaling analysis and data mining can validate or restructure baseline signaling states in pre-treatment samples of retrospective biorepositories and how they would respond to Osimertinib to further develop predictive pipelines for incoming patient's signaling therapy design to overcome acquired resistance. A multivariate model can provide regression statistics to link aggregated data (X signals) from all RapMIF and RapMPI experiments in all conditions to the outcome (Y response).

The predictive signaling response studies can benefit from tissue biopsies during and post-therapies. Once a large library (n>40) is collected, only RapMIF & RapMPI images in tissues can be analyzed using machine learning (e.g., Data clustering, tSNE & UMAP) and convolutional neural networks (CNN) on the results of clusters per tissue type. Using 70/30 data split, CNN can train and test the Osimertinib response in available prospective patient specimens, providing opportunities to implement deep learning for the prediction of combinatorial regimens from the signaling maps in tissues of NSCLC patients with EGFRm status and clinical outcome data.

Methods and scientific rigor are provided in individual aims to validate results using multiple complementary assays. The mean percent of individual cell types and single cells is compared between groups using t-tests or Mann-Whitney U tests or ANOVA with posthoc test. Spatial protein expression, network, or interactors are associated are tested with clinical, demographic, and pathologic variables of interest using Fisher's exact/chi-squared tests or Wilcoxon rank-sum/ANOVA tests for categorical and continuous variables, respectively. Survival is estimated using the Kaplan-Meier method and evaluate the impact of race, spatial signaling network, and interactome on survival using Cox proportional hazards models. Longevity, proliferation, and signaling expression levels on drug-sensitive and resistant cell lines or control samples in triplicate are analyzed with a linear mixed-effects model with fixed effects of 5 drug combinations, 4 concentrations, spatial neighboring cells, and their interactions with a random cell line effect to control for variability between cell lines. With 5 mice per combination of treatment and cell line, an effect size difference of 1.3 between any two treatment arms can be detected with 80% power and 0.05 significance level. All experiments are done in triplicate. >50% cell viability in untreated cells. >3-days cultures are used for cell viability. >4-replicates are used for each condition. SD and Mean (CV %) values can be used for comparisons. <0.05 p-value can be significant in cell counts. >80% cell confluency can be used for drug treatments. N, the number of cells can be normalized per drug condition. >1,000 cells per well can be imaged and analyzed. % fold change as designed in the drug concentration. >80% imaging reproducibility in experiments, >0.05 p-value in two-sided T-tests, >0.9 accuracies for pathology validation using Hematoxylin and Eosin (H&E) stain, >0.5 correlation between 30-markers in similar tissue types. Statistical >95% confidence using ANOVA with Bonferroni or Tukey's test can be used to compare spatial signaling network profiles of human and mouse tissues. Spatial signaling network feature analysis of tumors aims to achieve a power level of 0.95 based on a single false positive, desired 2-fold difference, and standard deviation of 1. The scientific rigor can be established by ensuring that variables such as age and sex are considered.

### Example 5—Rapid Multiplexed Immunofluorescence for Subcellular Spatial Protein Analysis

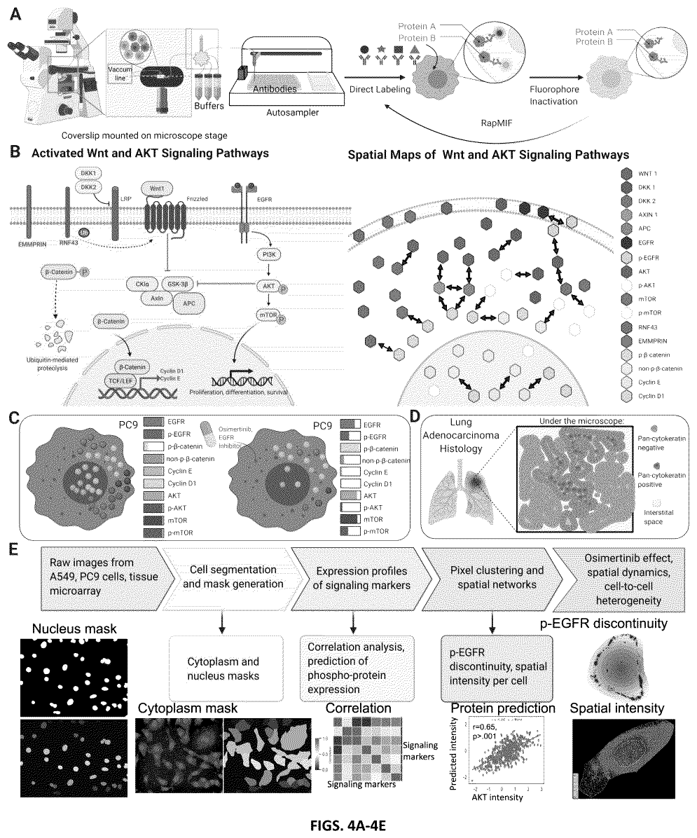

To resolve the subcellular spatial protein networks in the WNT/β-catenin and AKT/mTOR pathways, the presented RapMIF method was performed on A549 cells, EGFR-mutant PC9 cells, and lung adenocarcinoma tissues. RapMIF technology has been built upon the validated protocols of the CycIF method that typically utilizes robust fluorophore inactivation and repeated labeling of dyes to overcome the limitations of the microscopes constrained with 4-6 color channels in conventional IF (Lin et al., 2016). The RapMIF approach incorporates an autostainer setup and pre-conjugated antibodies to expedite the sample processing for highly multiplexed imaging of cells or tissues located on coverslips (FIG. 4A). Specifically, the RapMIF reduces the sample staining time from 12-16 hours to an hour. The RapMIF achieved imaging of up to 25 markers and revealed the signaling proteins as part of the WNT/β-catenin and AKT/mTOR pathways using quantitative expression profiles and spatial distributions of target biomarkers (FIG. 4B). The spatial signaling networks were altered upon Osimertinib treatment in PC9 cells (FIG. 4C). The RapMIF approach on tissue samples yielded signaling alterations in complex tumor microenvironments (FIG. 4D).

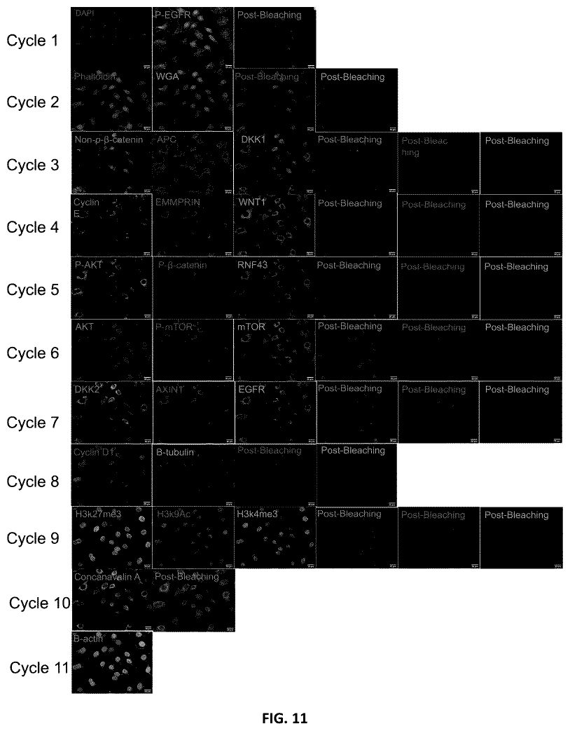

For the multiplexing experiment, RapMIF utilized a panel of 11 cycles, resulting in a total of up to 25 markers, including 5 structural, 17 signaling, and 3 epigenetic markers. For robust segmentation, the panel included Concanavalin A (Endoplasmic reticulum), Phalloidin (Actin), wheat germ agglutinin/WGA (Golgi and plasma membrane) as cell painting markers (Bray et al., 2016), and 3-actin (Actin filaments) and β-tubulin (Microtubules) to accurately delineate nucleus and cell body using an automated algorithm. To improve the staining dilution rate, titration was performed on antibodies. IF experiments evaluated antibody staining using two different dilution rates. The signal-to-noise ratios (SNRs) of these IF images was compared, yielding the condition with higher SNR used for RapMIF experiments (TABLE 1). The staining dilutions were consistent with the manufacturers' suggested dilution range. To reduce the time of sample incubation in indirect IF, most antibodies were conjugated with one of the 488, 555, and 647 Alexa dyes using Lightning-Link Rapid Alexa Fluor Antibody Labeling Kit (Novus/Abcam). These markers were evaluated in four conditions based on conjugated antibodies, unconjugated antibodies, overnight incubation at 4° C., and 1-hour incubation at room temperature (RT). The comparisons of antibody staining experiments at overnight and RT temperature incubation yielded similar signaling patterns, denoted in the cross-sections of plots across pixel histograms. For each round of staining, the sample was incubated with antibodies conjugated to distinct dyes for 1 hour at RT after blocking with cell staining media (CSM). Following imaging of labeled cells, the dyes were bleached in the NaOH—H₂O₂ mixture under white light for 1 hour (FIGS. 10A-10D). Although the base-hydrogen peroxide mixture bleaches Alexa 488, 555, and 647 at different rates, a common incubation for 30-45 min within a fluorophore inactivation buffer was sufficient (Lin et al., 2018). Post-bleaching images were acquired after the inactivation of the dyes before the next round of staining (FIG. 11). To confirm the bleaching efficacy, quantitative analysis was performed on post-bleach images of the A549 cells (FIGS. 12A-12C). Negligible cell loss was noticed in the first and second cycles in the sample, corresponding to the 2-5% and 0-2% of cells per cycle, subsequently (Lin et al., 2018). Following fluorophore inactivation, the coverslip was re-blocked with CSM to make the cells ready for the next round of staining. The details of customized parameter settings for fluid transfer and antibody incubation are included in TABLE 2. The 11-cycle RapMIF experiment yielded up to 25 protein-type images on the stained slides at the single-cell level. The multiplexed single-cell measurements revealed the subcellular distribution of signaling protein networks. Compared to CycIF, RapMIF has reduced the cycle time from 12 hours to 5 hours (TABLE 3). This prevents cell degradation, a common issue for the multiplexed experiments performed over long-time periods. To maximize the efficiency of epitope detection in tissue, overnight incubation for staining was performed, consistent with the settings in t-CycIF. Current experiments rely on the commercially available tissue on 1-mm thick glass slides, however, tissue can be sectioned on the thin coverslips to be compatible with the acrylic holder used for RapMIF automation (FIG. 10D).

To reduce the need for conjugated antibodies for each marker, both indirect labeling and direct labeling were combined in the panel. To minimize the effect of unspecific binding of secondary antibodies to conjugated primary antibodies, the indirect labeling of p-EGFR was designed in the first cycle. Indirect labeling of 0-tubulin and 3-actin allowed cell segmentation and pan-cytokeratin distinguished the cancerous tissue regions in the last cycle. The images were taken at 40× with a step size of 0.4-μm to reveal the slowly varying signaling molecules across the z-axis of a cell. The best focus images were generated from +/−2 z-stacks and they were used for post-processing of the single cells. To avoid the effect of the dye inactivation solution on Phalloidin labeled structures, p-EGFR targeting antibodies were directly incubated between the first and second cycles for the cell and tissue experiments without an intermediate bleaching step.

To study the spatial distributions of proteins, a single-pixel analysis was performed to demonstrate the nuclear and cytosolic expression levels and translocations of signaling proteins in WNT/β-catenin and AKT/mTOR pathways. The measurement of the expression level of each marker in two regions, cytosol and nucleus, was quantified separately (FIG. 4E). Pixel-level colocalization of signaling proteins was then performed by unsupervised clustering of the protein images in subcellular regions, revealing the spatially distinct signaling neighborhood regions (FIG. 4E). Machine learning (e.g., Random forest) analysis of subcellular maps and expression profiles of the signaling targets yielded the phosphorylation activity of four markers, p-AKT, p-mTOR, p-EGFR, and p-β-catenin, demonstrating the interdependency of spatial signaling networks that are altered in wild-type and drug-perturbed cells (FIG. 4E). Together, the RapMIF approach resolved the cell-to-cell heterogeneity in expression profiles and spatial signaling maps and deciphered the phosphorylation activities of the kinases and their direct role in regulating signaling pathways.

Discussion: Signaling pathways mediate cell communication, proliferation, differentiation, and migration. The pathways are usually regulated through protein complexes, controlled via PPIs. To avoid the need of modifying samples genetically, which is not feasible with patients' samples, proximity ligation assay has shown the capability of detecting the PPI in situ (distance <40 nm) at endogenous protein levels (Alam, 2018). Förster resonance energy transfer (FRET) has also been used for observing dynamics and reversible PPIs in vivo by transferring the fluorophores from a donor to an acceptor in its close proximity. Time-resolved FRET advances the current FRET methods with a faster, more sensitive, and less complex detection of PPIs (Maurel et al., 2008; Rajapakse et al., 2010). Due to spectral bleed and limited energy transfer efficiency, it is difficult to implement FRET with super-resolution imaging methods. Biomolecular fluorescence complementation (BiFC) has shown the feasibility of detecting PPIs based on fluorescent complementation reporters. Implementing BiFC on a super-resolution imaging approach, photoactivated localization microscopy (PALM), achieves imaging of the subcellular dynamics of PPIs at high spatial-temporal resolution (Liu et al., 2014).

Phosphorylation can be used to modulate PPIs, thereby regulating signaling transduction through either modifying proteins post-translationally or producing secondary messengers. Protein kinases play an important role in activating cellular processes, and the target proteins can be phosphorylated at different sites resulting in heterogeneous functions (Xue et al., 2012). Conventional methods for identifying kinase substrates suffer from several limitations. Kinase assay is one of the methods to identify kinase-substrate pairs by using analog-sensitive alleles. The entire in vitro substrate can be assayed for a particular kinase. Various synthesized analogs provided a panel for substrate identification and protein kinase function (Elphick et al., 2007). However, this method is not applicable when the introduced analog does not affect the kinase's function (Mok et al., 2009). Protein microarray analysis can also reveal the potential phosphorylation reactions as well as novel functions. To identify potential substrates, the protein kinases are added to arrays of immobilized proteins and generate signals upon detection (Meng et al., 2008). However, the relationships are identified in vitro and may not represent the relationships in vivo (Mok et al., 2009). Kinase substrate tracking and elucidation (KESTREL) has also been used to identify protein kinase substrates. The high background phosphorylation in the cell extracts can obscure the detection of putative phosphorylation, however, KESTREL achieves the identification of physiologically relevant substrates of proteins in crude cell extracts with an improved signal-to-noise ratio by limiting the phosphorylation reaction time and increasing concentrations of protein kinase of interest. Performing chromatography steps before the analysis could also greatly improve the SNR ratio. Also, to establish high physiological relevance, two or three closely related protein kinases with similar binding specificities were used for one substrate detection. However, the temporal and spatial information is largely ignored in the cell lysate (Knebel et al., 2001). However, the assays have experienced challenges with the background in cell extracts and medium-to-low specific activity to kinases (Knebel et al., 2001; Troiani et al., 2005).

High-throughput techniques such as mass cytometry have been widely used to identify the phosphorylation sites of the target protein. Although the bulk level correlation between the protein expression levels of markers was previously mapped out, the spatial correlations and the precise association between the kinases and their direct substrates are still unclear (Xue et al., 2012). Also, the cell-to-cell heterogeneity in the signaling transduction resulting from factors crucial for cancer development could not be systematically profiled due to technical limitations (Lun and Bodenmiller, 2020). The study could advance the current single-cell mass cytometry (CyTOF) in detecting kinases involved in signaling pathways using additional antibodies in the multiplexed imaging data (Han et al., 2015; Leelatian et al., 2015; Mingueneau et al., 2014). RapMIF provides spatial information on the protein distributions compared to traditional protein analysis assays. Since kinase assay requires the synthesis and introduction of ATP-analogs and is only applicable when the introduced analogs do not interfere with kinase’ function (Mok et al., 2009), RapMIF circumvents the complex process of synthesizing ATP-analogs by directly detecting protein activity from multiple cycles of IF. Also, unlike KESTREL, RapMIF conserves the subcellular structure by avoiding lysate in the cells.

The multiplexed single-pixel analyses have quantified the subcellular protein correlations, indicating potential direct physical neighborhood among signaling protein subsets. Distinct multiplexed intensity profiles of signaling markers revealed the crosstalk between heterogeneous signaling pathways. The presented RapMIF method simplified multiplexed protein analysis by pre-conjugating the antibodies with commercially available dyes. Also, it was observed that the bleaching solution interfered with Phalloidin signals in A549 cells. Therefore, in the A549 data analysis, a combination of p-EGFR, Concanavalin A, B-actin, WGA, APC, and WNT1 markers was used to outline the cell boundary for segmentation. Also, to avoid the bleaching effect, cycle 2 (Phalloidin and WGA) staining was done directly after cycle 1 (p-EGFR) without an intermediate bleaching step in PC9 multiplexing samples. A549 cells showed a larger cytosolic area than that of PC9 cells, providing a more detailed spatial network model for studying the protein distribution in the cytosol. The spatial maps demonstrated the translocation patterns of signaling markers corresponding to their functions in both cell types. The combination of ERK or MEK inhibition with Osimertinib could delay or overcome the acquired Osimertinib resistance (Gu et al., 2020a). Thus, the spatial signaling patterns in the MEK/ERK pathway, another downstream pathway of EGFR, regulating cell proliferation and survival could further be determined using additional antibody targets.

Even in the same population of cells, the cells may display heterogeneous signaling profiles due to the difference in signaling-acting pathways. Autocrine signaling involves the production and secretion of an extracellular mediator, acting on the same cell, while paracrine signaling occurs between different types of cells. Both signaling pathways mediate tumor growth, invasion, and metastasis. During cancer development, the growth factors are usually produced in an autocrine manner, allowing the cell to simulate itself in positive feedback (Caicedo, 2013; Ungefroren, 2021). Current methods have been developed to decipher the cell-cell communication in single cells, however, they failed to demonstrate the heterogeneity in autocrine and paracrine signaling interactions. CellChat as a predictive model for cell-cell communication has been achieved to study the autocrine-acting vs. paracrine-acting pathways in the same cell type or same subpopulation of cells. However, this approach highly relies on the accuracy of the ligand-receptor databases and it produces fewer interactions than other methods when detecting communications from distant cells (Jin et al., 2021). Therefore, combining predictive models and proteomics could optimize cell-cell communication methods. Also, the complex crosstalk among signaling pathways could contribute to the heterogeneity in signaling profiles. Fluorescent reporters can be introduced and utilized to track protein activity in the subpopulation of cells following drug treatment (Kurppa et al., 2020). However, the current study focuses on the design of signaling pathway analysis using molecular snapshots, instead of the subpopulation validation in real-time.

RapMIF multiplexed 17 signaling markers and converted their distributions into 33-pixel spatial clusters. Physical proximity analysis between two distinct proteins yielded a total of 272 (17 choose 2 combinations) possible pairwise neighborhoods, yielding their correlations in cytosolic and nuclear expression levels at the single-pixel level. Pairwise analysis of protein neighborhoods could reveal potential critical signaling proteins or networks (Ramana, 2019). Besides, coordination of the multi-protein neighborhood can be extracted from the multiplexed signaling data. For instance, the tri-protein interactions in cytosol and nucleus are assessed with 1360 (17 choose 3 combinations) possible three-way neighborhoods. Combinatorial signaling analysis would predict the dynamics of signaling networks when multiple proteins physically interact, a task that would be challenging to achieve using bulk signaling assays.

The system design achieved a fully automated staining system including blocking, staining, imaging, and bleaching. The fluidic automation takes the antibody diluted in CSM media in the multi-well in a 2.5-fold dilution. Therefore, to save the antibody volume, the antibody loading step by step could be done by manually pipetting the antibodies to the sample. If needed, the automated system could be further optimized to reduce the dilution rate of the antibody during the staining step. Aspiration settings and washing speed could be adjusted to minimize cell loss across cycles. The p-EGFR signaling receptors in PC9 cells were dependent on the sample quality due to the step of drying the coverslip before mounting it to the holder. The freshness of the sample may affect the morphology of p-EGFR. Thus, the multiplexing experiments in PC9 cells were initiated on the same day of cell fixation to make sure the quality of the samples. Thus, the RapMIF could provide a precision oncology framework for defining the signaling dynamics in both intensities and spatial distribution of human cancers.

For multiplexing experiments, two factors are concerned to ensure cell quality, the efficiency of the bleaching approach and the total spent time for one set experiment. RapMIF utilizes fluorophore-conjugated antibodies for direct labeling. Fluorophore-conjugated antibodies generally produce relatively weaker signals than indirect labeling. However, indirect labeling raised some problems in the context of multiplexed experiments. It is constrained by the availability of the reactivity of secondary antibodies to limited species (e.g., mouse, human, donkey). Also, multiplexing indirect IF requires antibody stripping rather than a regular bleaching approach. The harsh process may cause cell loss and tissue detachment. Compared to the antibody stripping approach removing the whole secondary antibody, the RapMIF bleaching approach is effective in removing dyes while preserving the cells. One concern of the RapMIF is whether the direct IF staining at RT for 1-h for each cycle can label all the antigens in the cell. This trade-off can be addressed by improving the antibody concentration to generate similar signals in 1-h RT staining compared to overnight incubation. The approach aims to achieve rapid profiling of signaling pathways while maximizing the efficiency of protein detection. The staining settings were kept consistent across different samples, making it reliable for studying drug efficiency. Also, staining the sample for 1 hour instead of overnight reduces the 12-cycle multiplexing experiments from 24 days to 6 days, and prevents cell degradation. Another drawback of the RapMIF on FFPE tissues is that FFPE tissue generates a high autofluorescence background and is affected by shading or vignetting due to imaging artifacts. Signaling proteins are expected to express nearly everywhere, making it harder to distinguish between background and real signals. Therefore, background correction steps were performed using the BASIC algorithm to reduce the contribution of background on real signals (Peng et al., 2017). Also, post-bleach imaging data serve as a control of native autofluorescence signals and confirm the background corrected images using the stained raw tissue image that is subtracted from the post-bleach image.

In terms of antibody specificity validation, a positive control (e.g., a cell line expressing the target) and negative control (e.g., a cell line not expressing the target) can be utilized to confirm the selective binding of the antibody. Sometimes, it is difficult to find cell lines that do not express the target. Therefore, knock-out (KO) models such as CRISPR-Cas9 and siRNA can serve as negative controls, and be applied to various approaches, including mass spectrometry, western blot, IHC, and IF (Kurppa et al., 2020; Uhlen et al., 2016). In previous studies, independent antibody strategies are also suggested to confirm antibody specificity. When two antibodies bind to different epitopes of the same target, they can yield correlated signals, suggesting both antibodies recognize the same target (Uhlen et al., 2016). However, this is beyond the scope of current work and independent validations of antibody specificity are lacking in the current study. In the study, the antibody staining was evaluated using conventional IF protocol before performing the multiplexing to improve the staining quality of the antibodies.

Another important consideration is the physical dimensions of signaling protein visualizations. The diffraction limit of light microscopy is around 200 nm, while the antibody size is around 10 nm (Erickson, 2009; Tan et al., 2008; Xing et al., 2016). Due to the low spatial resolution, standard light microscopy fails to resolve the protein complex interactions based on the fluorescent signals. To determine the colocalization of two proteins, their spatial distributions are needed. When two proteins are colocalized with one another, the overlay of the two spatial distributions should show a correlation of intensity at the pixel level. In this case, the pixel size is around 188 nm using an oil immersion 40× objective lens with a 1.4 numerical aperture and the protein molecule is around 2-9 nm (Erickson, 2009). For a single cell, the number of protein molecules is a median of 170,000 (Li et al., 2014) to 2×10⁹, considering the number of ribosomes, tRNA, synthesis rate, and translation rate, among others (Princiotta et al., 2003). Each pixel would contain around a few to 15,700 proteins. Thus, due to the limitation on the resolution of the microscope, it is unable to resolve the single protein interaction. Additional color deconvolution methods might be needed to reach single protein detection. The pixel-level correlations can distinguish the random color overlap due to protein compartmentalization from protein colocalization and indicate that true colocalization only occupied 3% of total colocalization (Costes et al., 2004). To remove nonspecific interactions in data analysis, a probabilistic protein interaction network has previously been developed to identify protein interactions based on the normalized spectral abundance factors using Bayes' approach. The probabilistic model predicts each pair of proteins by providing quantitative information on the preference of each interaction. It takes advantage of quantitative proteomics by measuring spectral counts to generate a probabilistic network of protein interactions, providing an alternative framework to quantify probabilistic pixel-level protein interactions in the RapMIF data (Sardiu et al., 2008). While it is not implemented, it can provide an alternative data analysis approach to quantify probabilistic pixel-level protein distributions in the same pixel area in the RapMIF data. Current studies focus on the development of the RapMIF pipelines for studying the crosstalk of multiple signaling pathways and the protein neighborhood likelihood using spatial networks by studying the neighboring protein distribution in the same pixel area. However, super-resolution can be implemented in this technology to further resolve protein complexes. Protein interaction detection assays, such as proximity ligation assays, can be multiplexed to achieve multiparameter detection of PPIs.

### Example 6—Highly Multiplex Spatial Decoding of WNT/β-Catenin and AKT/mTOR in A549 Cells

To optimize the staining protocols and data analysis pipeline, RapMIF was performed to quantify the expression and spatial distribution of signaling markers involved in WNT/β-catenin and AKT/mTOR pathways in the A549 NSCLC cell line (FIGS. 5A-5F). The raw IF images (FIG. 5B) were processed for cell segmentation and quantification of the protein expression levels (FIGS. 5C-5D). To consider the cell morphology difference, the mean intensity was used for correlation analysis (FIGS. 5E-5F), and total intensity correlation also showed the location-dependent intensity comparison. The analysis of WNT/β-catenin and AKT/mTOR pathways in stabilizing β-catenin demonstrated that non-p-β-catenin was positively associated with the expression of cyclin E and cyclin D1 (R=0.6˜0.7). This finding indicated non-p-β-catenin's role in the progression of the cell cycle (FIG. 5E). The co-expression of cytosolic protein distributions was detected in non-p-β-catenin, AKT, and p-AKT signaling markers (FIG. 5F). It is speculated that the p-AKT can promote the activity of β-catenin by inhibiting GSK30, one member of the β-catenin degradation complex (McCubrey et al., 2014b). The stabilization of β-catenin by p-AKT could then facilitate the activity of cyclin E and cyclin D1 in the nucleus. The pairwise analysis between two proteins determined their correlation in expression levels, yielding 272 pairs in cytosol and nucleus of individual cells. The possibility of using 3-way intensity correlations to illustrate the 3-protein neighborhood in single cells was also shown. The expression level of cytosolic p-mTOR proteins yielded a higher correlation with cytosolic p-β-catenin than with cytosolic non-p-β-catenin in the A549 cell line. This pattern may be due to the role of mTORC1 in suppressing the WNT/β-catenin pathway by inhibiting LRP6 phosphorylation and FZD activation (Zeng et al., 2018). To confirm the removal of fluorescence signals, the marker correlations of single-cells was demonstrated from the post-bleach images.

To capture the spatial information among the two pathways, signaling protein maps of the multiplexed panel were converted into 33-pixel clusters and uniquely colored back onto the original images of the single cancer cells (FIGS. 6A-6D). Each pixel cluster corresponded to a set of signaling markers co-expressing in a pixel distribution based on similarities of multiplexed intensity profiles (Gut et al., 2018). Background pixels were filtered out based on semi-automatically gated parameters. The UMAP visualizes pixel-wise clusters in A549 cells (FIG. 6B). It was observed that cluster 13 has relatively higher expression levels of WNT1 and p-AKT, indicating the potential neighborhood between the two pathways (FIG. 6D). The pixel clustering map demonstrated that non-p-β-catenin was distributed in the nucleus in cluster 2, while another portion of the signal was detected in the cytosol and along the cell membrane in cluster 16 (FIG. 6D). Such subcellular signaling patterns may indicate non-p-β-catenin functions as a component of cell-cell adhesion structures by interacting with E-cadherin and actin (Kase et al., 2000). Besides, p-β-catenin (Ser45) was highly expressed in the cytosol in cluster 21 of A549 NSCLC cells (FIG. 6B), supporting the degradation of the phosphorylated β-catenin in the cytosol, but not in the nucleus. Cytosolic p-β-catenin degradation is dependent on the phosphorylation at Ser33/Ser37/Thr41 by GSK30 (Li et al., 2012; Parker et al., 2020). The p-β-catenin signaling has previously been found almost exclusively expressed in the nucleus in melanoma and colorectal cancer cells, while p-β-catenin has been detected in both cytoplasm and nucleus in invasive breast carcinomas (Chung et al., 2001; Kielhorn et al., 2003; Sinnberg et al., 2011). The presence of p-β-catenin in the nucleus may be due to the overexpression of p-β-catenin. The overexpressed p-β-catenin weighs much larger than the proteins undergoing degradation, thereby leading to the translocation to the nucleus (Nakopoulou et al., 2006). The p-EGFR (Tyr1086) signaling proteins were mainly distributed in cytosol shown in clusters 14 and 32, while some activity was detected in the nucleus denoted in cluster 2 (FIG. 6D). This translocation pattern is possibly due to the activation of EGFR at different phosphorylation sites. This can facilitate the translocation of EGFR to the nucleus, whereas nuclear EGFR functions differently from the EGFR on the cell membrane, for instance, to promote the transcription of cyclin D1 (Brand et al., 2011, 2013).

The colocalization of WNT/β-catenin and p-AKT pathway components was also observed in the spatial signaling network maps, demonstrating the physical neighborhood between two pathways in the same pixel area (FIG. 6D), providing the common downstream effectors. Both pathways can cause the stabilization, and accumulation of β-catenin and increase the activity of cyclin D1 and cyclin E to facilitate the progression of the cell cycle at the single-pixel level. In addition to the indirect stabilization of β-catenin by p-AKT via GSK30, p-AKT and non-p-β-catenin may also have potential direct neighborhood due to the coexpression and colocalization of the two proteins (FIG. 5E, FIG. 6D). AKT can phosphorylate β-catenin at Ser552 directly, causing β-catenin to dissociate from cell contacts, and promote its translocation from cytosol to nucleus to increase transcriptional activity and tumor cell growth (Fang et al., 2007).

Next, a machine learning model was designed to predict the β-catenin phosphorylation activity based on the 20 biomarkers using a Random forest (RF) algorithm (FIGS. 6E-6G). The activity of β-catenin is highly correlated with cell growth, proliferation, and migration. There is no reliable biomarker to predict the activity of β-catenin. In the prediction model, the subset of signaling proteins including WNT1, RNF43, and AKT exhibited relatively high importance in predicting the phosphorylation of β-catenin, yielding an r=0.76 fit (p<0.001) in prediction accuracy. The same RF model provided predictions of other phosphorylated markers, including p-AKT, p-mTOR, and p-EGFR. The multiplexed dataset revealed potential biomarkers as features to predict the phosphorylation activity of β-catenin.

Cell cycle analysis was also conducted based on the quantification of nuclear cyclin D1 expression level, classifying the cells into G1, S, and G2 phases (FIG. 6H). Most of the cells were harvested in the G1 phase. This indicated the regulatory role of cyclin D1 in hyper-phosphorylating the retinoblastoma tumor suppressor protein (Rb1) by forming cyclin D1/CDK4 complex, necessary for the cell to enter the S phase in the cell cycle (Gautschi et al., 2007). The number of cells expressing cyclin D1 was low during the S and G2 phases due to the exportation of cyclin D1 from the nucleus to the cytoplasm for degradation by the proteasome (Guo et al., 2005). Similarly, cell cycle analysis was assessed based on cyclin E expression levels. Cyclin E demonstrated similar results that most cells expressing cyclin E were harvested in G1, and some in S phases, indicating its role in promoting G1 and S phases for cell proliferation and growth by binding to CDK2 (Hwang and Clurman, 2005). In a study, cyclin E accumulated around the nuclear membrane and was mainly distributed in the cytosol (FIG. 6D). The accumulation of cyclin E in the cytoplasm may be the result of the formation of low molecular weight cyclin E (LMW-E) with the loss of the canonical NH2-terminal nuclear localization sequence. The overexpression of LMW-E is associated with breast cancer recurrence and poor overall survival; it has also been observed in NSCLC (Hunt et al., 2017; Koutsami et al., 2006). Besides, the role of DKK1 in NSCLC was also explored. DKK1 plays multiple roles in different diseases; it functions as an oncogene in hepatocellular carcinoma, liver cancer, prostate cancer, and myeloma (Kagey and He, 2017; Zhang et al., 2017). However, it can also promote epithelial-mesenchymal transition (EMT) by stabilizing β-catenin and inducing its nuclear localization (Zhang et al., 2017). In the RapMIF data, the nuclear DKK1 proteins exhibited a higher correlation to non-p-β-catenin than p-β-catenin in both cytosol and nucleus. This reveals that the nuclear DKK1 may act as a promoter in the WNT/β-catenin pathway. However, cytosolic DKK1 yielded a similar correlation to cytosolic p-β-catenin and non-p-β-catenin. The modulation of DKK1 in cancer cell development may not be limited to WNT signaling pathways. Cytoskeleton-associated protein 4 (CKAP4) has been found as a novel receptor of DKK1. The binding of DKK1 to CKAP4 could activate PI3K/AKT pathways, increasing cell proliferation (Kagey and He, 2017). Therefore, the role of DKK1 in the WNT/β-catenin pathway may be location-dependent, and further studies are needed to decipher the function of DKK1 in NSCLC.

### Example 7—Spatial Networks in EGFR Mutant Cells for Analysis of EGFR, WNT, and AKT-mTOR Crosstalks

To reconstruct the signaling networks in EGFR mutant cells under drug perturbations, the RapMIF was used to profile multiple signaling molecules in EGFR mutant NSCLC-sensitive PC9 cell lines. Osimertinib demonstrated a prominent effect in inhibiting PC9 cells, and the cell survival rate decreased as the concentration was increased from 0 to 10-nM under a 3-day treatment (Gu et al., 2020b, 2020a). PC9 cells responding to Osimertinib treatment can develop the acquired resistance. However, in previous work, the western blot experiments of signaling proteins in PC9 cells treated with the combination of Osimertinib and either ACK1 inhibitor or MEK inhibitor exhibited effectiveness to overcome the acquired resistance to Osimertinib (Gu et al., 2020b). Besides, it showed that Osimertinib can effectively inhibit the p-AKT (S473) level in PC9 cells, but not AKT itself. Similar results were obtained from the signaling studies in H1975 cells, a drug-resistant cell line (Gu et al., 2020c). Osimertinib inhibited the phosphorylation of EGFR in a phosphorylation site-dependent manner and decreased the p-EGFR (Y1173) level but failed to alter the p-EGFR (Y1068) in PC9 cells (Gu et al., 2020b, 2020a). However, Osimertinib could affect the downstream effector of EGFR, Grb2, by suppressing its binding to p-EGFR (Tyr1086) in H1975 cells (Lee et al., 2018).

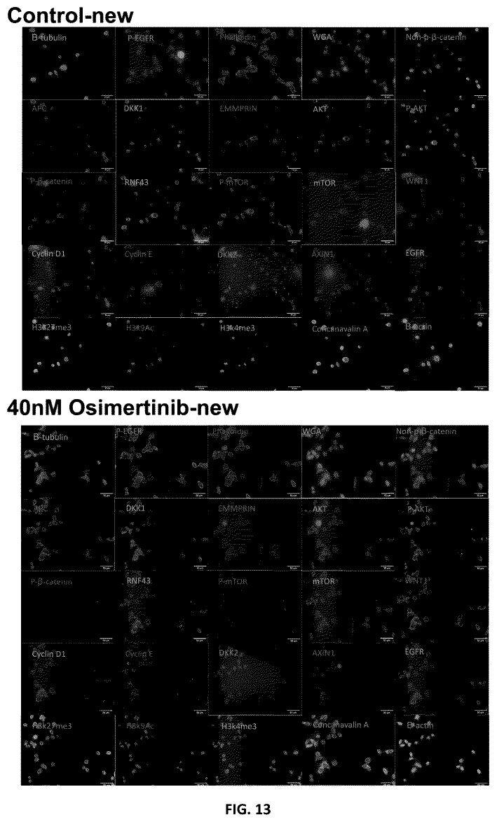

In a study, it was first asked whether the EGFR pathway would be inhibited by Osimertinib in PC9 cells, eventually affecting the proliferation and growth of the PC9 cells. A 48-hour Osimertinib treatment reduced the number of adherent PC9 cells compared to untreated ones. Higher drug concentrations further decreased the cell viability compared to lower drug treatments. Following drug treatments, the RapMIF was used to generate the multiplexed imaging maps of up to 25-plex signaling proteins in PC9 with and without the treatment of 40 nM Osimertinib (FIGS. 7A-7B, FIG. 13). The drug treatment disrupted spatial continuity in the p-EGFR distributions on the cell membrane (FIGS. 7A-7B). This finding may be due to the effect of Osimertinib on inhibiting the dimerization and phosphorylation of EGFR. Furthermore, the p-EGFR signaling proteins were highly expressed in both cell membrane and nucleus in the absence of Osimertinib. However, p-EGFR was mainly localized in the cell membrane and the nuclear p-EGFR expression was relatively low under the drug treatment (FIGS. 7A-7B). Similar trends were observed in p-EGFR validations across four different drug concentrations. As a positive control, β-tubulin exhibited uniform distributions across the cytoplasm of the cell with and without drug treatments. In addition to staining in the cytosol, the 0-tubulin expression level was enriched in the nucleolus due to the role of 0-tubulin in the mitotic spindle in helping nucleolus division at mitosis or nonspecific staining in the nucleolus (Jouhilahti et al., 2008; Walsh, 2012). The nuclear p-EGFR proteins are typically shed from the cell membrane in the sample without Osimertinib. The translocation of p-EGFR occurs via a nuclear importing system, and the nuclear p-EGFR can promote cell proliferation in an EGF-dependent manner to control the cyclin D1 gene expression (Lin et al., 2001; Nishimura et al., 2008). Also, previous literature suggested that the p-EGFR yields a high correlation to the cells with high proliferation in native tissues (Hoshino et al., 2007; Lin et al., 2001). Thus, the Osimertinib treatment not only inhibited the phosphorylation of p-EGFR at the cell membrane but also reduced the translocation of p-EGFR to the nucleus, impeding cell proliferation.

To evaluate the effect of Osimertinib on EGFR's downstream proteins, it was investigated whether p-EGFR would correlate with p-AKT expression level. The p-EGFR signaling proteins demonstrated a high correlation to p-AKT after drug treatment based on the mean-intensity correlation of single-cells (FIGS. 7C-7D), suggesting the role of Osimertinib in inhibiting both EGFR and the downstream targets of AKT/mTOR pathways. Furthermore, the p-EGFR expression decreased as the drug concentration increased from 0 to 20 nM to 40 nM (FIG. 7E), whereas p-EGFR exhibited relatively higher expression under 60 nM than 40 nM. This may be due to concentration-dependent drug response, causing the discontinuity in receptor distributions and downregulation of the p-EGFR expression. Interestingly, the p-EGFR (Tyr1086) expression level remained minimally altered under the Osimertinib treatment (FIG. 7F). This may be due to the difference in phosphorylation sites as a different site of p-EGFR (Y1173) was downregulated upon Osimertinib treatment (Gu et al., 2020b). Therefore, multiple phosphorylation sites of p-EGFR can contribute to the effect of Osimertinib in inhibiting p-EGFR. More importantly, p-AKT was consistently downregulated (FIG. 7G), demonstrating the function of Osimertinib in decreasing the downstream pathway of p-EGFR (Gu et al., 2020b).

Next, it was asked whether Osimertinib would alter the WNT pathway in PC9 cells. Prior studies on breast tumors and HC11 mammary epithelial cells yielded constituent activation of WNT1 to transactivate the EGFR pathway by increasing the ligand availability (Civenni et al., 2003; Faivre and Lange, 2007; Schlange et al., 2007). However, in the RapMIF results, WNT1 showed a low correlation with p-EGFR in both conditions, whereas a high correlation with p-AKT in the presence of the drug was obtained (FIGS. 7C-7D). This observation indicated that the activation of WNT1 would not act on EGFR directly, but could transactivate EGFR downstream targets. Also, the drug-treated cells exhibited a higher positive correlation between p-EGFR and non-p-β-catenin compared to the control (FIGS. 7C-7D), and the Osimertinib treatment increased the WNT1 signaling (FIG. 7F). Thus, the inhibited EGFR pathway could promote the activity of WNT signaling and β-catenin under the Osimertinib treatment.

The effect of Osimertinib in spatial distributions and networks in PC9 cells was then analyzed. The spatial signaling network maps of the PC9 cells after drug treatment exhibited more dispersed distribution in the cytosol, noted as clusters 0, 1, and 2, compared to the control sample (FIGS. 8A-8C). Subsets of spatially resolved clusters exhibited high expression levels of WNT1, indicating that inhibition of the AKT pathway could increase the activity of WNT1 as a compensatory pathway. Subcellular spatial signaling networks were represented as a physical neighborhood map for the assessment of protein-protein proximity at the pixel level (FIG. 8D). Each node corresponds to one of the most expressed signaling markers in that cluster of pixels (FIG. 8E). The color of the node matches the cluster color assigned to the pixel's identity. The length of the connected line represents the pixel-to-pixel closeness between two clusters. The color of the line demonstrates the probability of neighboring with another cluster. The size of the node shows the percentage of the cluster out of all clusters. For instance, Cluster 2 (WNT1) and cluster 6 (non-p-β-catenin) occupied a large proportion (frequency) after drug treatment, indicating the Osimertinib inhibited the AKT pathway while upregulating the WNT pathway. Also, cluster 6 (non-p-β-catenin) was mainly distributed in the nucleus, indicating the location of non-p-β-catenin. Cyclin D1 was highly expressed in cluster 14, indicating the activation of cyclin D1/CDK4 complex in the cytoplasm to promote the G1 phase and cell proliferation (Gladden and Diehl, 2005). Another interesting finding was that the degree of the node of WNT1 in cluster 2 reduced from 10 to 4 under the drug treatment. This phenomenon may be due to the effect of Osimertinib in reducing the neighborhoods and clustering between the WNT and AKT pathways. The p-β-catenin in cluster 12 and cluster 19 exhibited a higher degree of a node and more neighborhoods with p-EGFR clusters under the drug treatment. These observations indicated that the production and degradation of most p-β-catenin resulted from the EGFR pathway. The p-AKT displayed consistent downregulation in the replicates of multiplexed signaling experiments. Therefore, RapMIF data supported the role of the WNT1 pathway as a compensatory pathway, contributing to drug resistance.

### Example 8—Subpopulation Analysis on NSCLC Cells

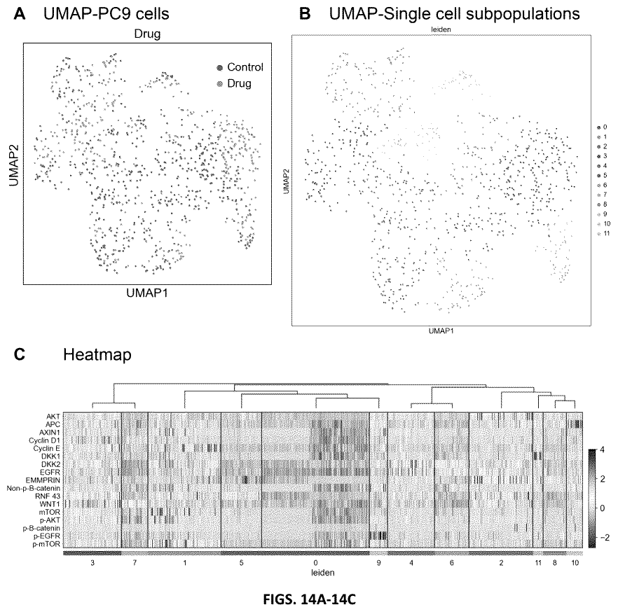

To visualize the heterogeneity of signaling expression levels, subpopulation analysis was performed on both EGFR-wild type (A549) and EGFR-mutant (PC9) NSCLC cell lines. First, A549 was clustered into 12 subpopulations based on their single-cell mean intensity levels. Each subpopulation represented a distinct signaling profile across 17 signaling markers. A549 cells in subpopulation 3 displayed relatively lower expression levels of all signaling proteins. In contrast, cells in subpopulation 0 and subpopulation 6 showed higher signaling expression levels and highly expressed WNT1, p-EGFR, cyclin E, mTOR, p-AKT, and non-p-β-catenin, indicating these cells may have higher activation of WNT/β-catenin and AKT/mTOR signaling. To decipher the cell heterogeneity, a similar analysis was performed on PC9 cells in the presence and absence of drug treatment (FIGS. 14A-14C). The single cells were organized into 12 subpopulations based on their single-cell mean intensity. PC9 40 nM Osimertinib-treated cells were mainly distributed in subpopulation 3 and subpopulation 0. However, the two populations displayed distinct signaling profiles. Subpopulation 3 demonstrated a signaling profile with high expression levels of 17 signaling markers, while the cells in subpopulation 0 have relatively low signaling expression levels. This population structure suggested that cells can exhibit various signaling profiles even after drug treatment. Therefore, dissecting the heterogeneity in the signaling distributions of cells in response to drug perturbations is necessary for combinational therapy design.

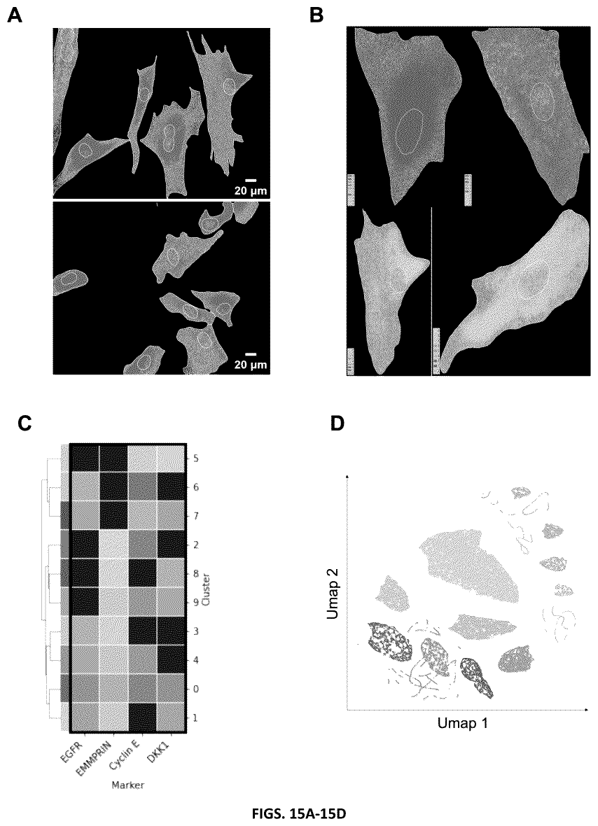

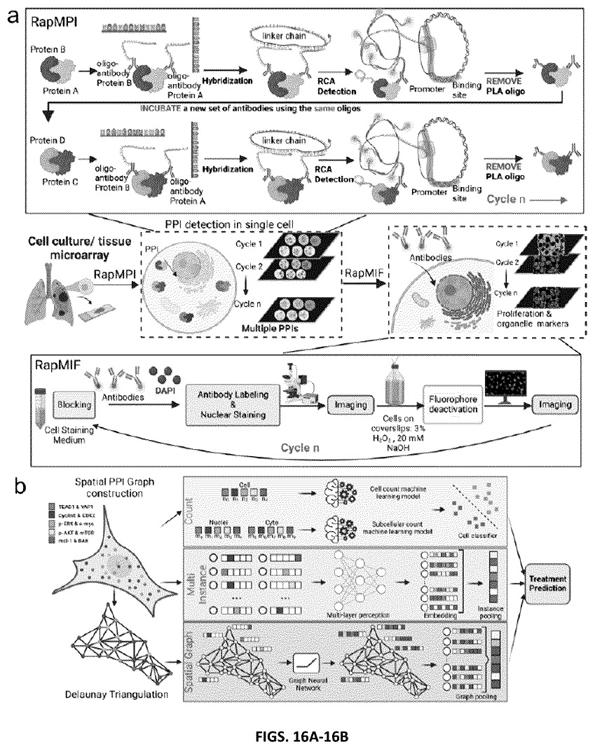

The signaling network concept and subpopulation analysis can be extended to other cell types. Thus, RapMIF examined a 7-plex protein panel in Umbilical cord mesenchymal stem cells (UC-MSC) (FIGS. 15A-15D). The more stretched cells provided better visualization of the signaling networks in the cytoplasm. Since WNT and AKT pathways also play an important role in cell differentiation and development, it would be worthwhile to study the signaling networks in MSCs for stem cell-based therapeutic designs (He et al., 2021; Takam Kamga et al., 2021)

### Example 9—Cross-Scale Analysis of Spatial Signaling Networks in Lung Tissues

To further verify the feasibility of signaling measurements in situ, RapMIF on FFPE tissues was investigated. The RapMIF on cell culture resolved the subcellular distribution among target proteins. However, pixel-level signaling analysis in tissues was challenging due to the densely packed cells with a limited spatial barcoding space in the cytosol of each cell. Thus, single-cell signaling maps were generated in tissues to decipher the architecture of normal and diseased cellular distributions. Haematoxylin and eosin (H&E) staining and Immunohistochemistry (IHC) have been widely used to diagnose disease (Lin et al., 2018). However, the information from H&E is limited, and IHC is a single-channel imaging method. Tissue imaging typically requires intricate spatial analysis for feature extraction and cell classification algorithms due to the native tissue architecture. The RapMIF made it possible to study potential signaling imaging biomarkers to predict drug response using the baseline tissues obtained from the patients before targeted therapies.

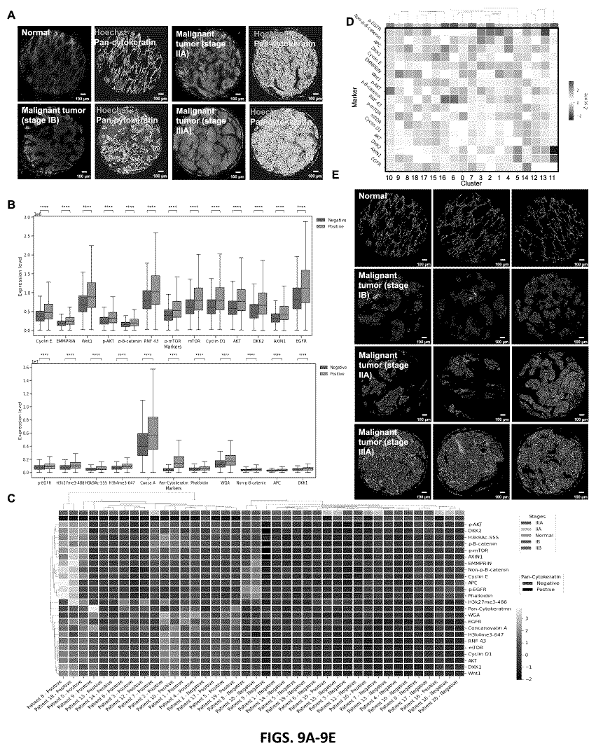

RapMIF screened up to 25-plex signaling proteins in a tissue lung adenocarcinoma microarray (BS04081a, Biomax) that contained 63 cores, 21 patients' cases, varying from normal, stage I, II, and III tumors. The images were first processed to correct the line artifacts caused by the uneven illumination from the flat field and dark field using the BASIC function in ImageJ (Peng et al., 2017). Background corrections improved the downstream image analysis. Single cells were then segmented in the tissue cores and classified into pan-cytokeratin positive and negative regions (FIG. 9A). Patients with higher tumor stages demonstrated upregulated WNT and AKT pathways with larger variation in the pan-cytokeratin-positive region (FIG. 9B). The constituent activation of the WNT and AKT pathway promotes the stabilization of non-p-β-catenin and the degradation of p-β-catenin (Jin et al., 2017). However, upregulated p-β-catenin was observed in tumor regions (FIG. 9B). The Crosstalk mechanisms between the WNT pathway and other pathways, such as JAK-STAT and NF-κB signaling pathways, may contribute to the upregulation of p-β-catenin (Bai et al., 2017; Ma and Hottiger, 2016). Also, the activity of β-catenin is phosphorylation site-dependent. Of note, β-catenin is phosphorylated at Thr41, Ser37, and Ser33 residues by GSK3, and then at Ser45 by CKI to undergo ubiquitination and degradation by the proteasome (Li et al., 2012).

Next, the signaling proteins were assessed across various cancer stages. Multiplexed signaling maps across 55 cores were classified by cancer stages and pan-cytokeratin expression at the single-cell level. The signaling markers in pan-cytokeratin-positive regions exhibited higher expression levels than normal tissue regions (FIG. 9C). The majority of signaling markers, RNF43, EGFR, H3K27me3, WNT1, p-EGFR, AKT, mTOR, and cyclin D1, demonstrated upregulation in the tumor regions. The variances of signaling expression levels were larger in pan-cytokeratin-positive regions, especially in stage IB tissues. However, there was no obvious trend in the signaling levels across different stages of the tumor. For patients 8 and 18, the expression levels of EMMPRIN, non-p-β-catenin, p-EGFR, APC, and cyclin E were higher in both pan-cytokeratin-positive and -negative regions.

To investigate the spatial distribution of signaling proteins, a single-cell clustering analysis was performed in the pan-cytokeratin-positive areas. The malignant tissue exhibited more variance in signaling expression from the same patient (FIGS. 9D-9E). Five distinct clusters were highly distributed in the malignant IIIA tumors, while only one cluster was mainly expressed in the normal tissues (FIG. 9E). In malignant stage IIIA, the sample was dominated by clusters 15 and 16, corresponding to the high expression levels of non-p-β-catenin, cyclin E, p-AKT, and p-mTOR. This finding indicated the upregulation of both WNT and Akt pathways. The signaling maps were similar in the pan-cytokeratin positive regions of normal and malignant stage IIA tissue (cluster 12, 14 respectively), however, the tissues presented higher expression of p-EGFR and non-p-β-catenin in malignant stage IIA, revealing the upregulation of AKT pathways (FIGS. 9D-9E).

The next step of RapMIF in tissues would be to study how AKT/mTOR and WNT/0-catenin pathways coordinate in wild-type, EGFR mutant FFPE tissues, and fresh frozen tissues. Fresh frozen tissues may provide varying staining results, dependent on the conditions of fixation and antigen retrieval (Shi et al., 2008). Signaling networks in tissues of EGFR mutant patients in response to EGFR TKI treatments would help predict the drug response and isolate signaling mechanisms responsible for drug resistance. Considering the complexity of tumor samples, combinations of additional signaling targets would enhance the prediction accuracy of patient classification using deep learning methods.

Discussion: Signaling pathways mediate cell communication, proliferation, differentiation, and migration. The pathways are usually regulated through protein complexes, controlled via PPIs. To avoid the need of modifying samples genetically, which is not feasible with patients' samples, proximity ligation assay has shown the capability of detecting the PPI in situ (distance <40 nm) at endogenous protein levels (Alam, 2018). Förster resonance energy transfer (FRET) has also been used for observing dynamics and reversible PPIs in vivo by transferring the fluorophores from a donor to an acceptor in its close proximity. Time-resolved FRET advances the current FRET methods with a faster, more sensitive, and less complex detection of PPIs (Maurel et al., 2008; Rajapakse et al., 2010). Due to spectral bleed and limited energy transfer efficiency, it is difficult to implement FRET with super-resolution imaging methods. Biomolecular fluorescence complementation (BiFC) has shown the feasibility of detecting PPIs based on fluorescent complementation reporters. Implementing BiFC on a super-resolution imaging approach, photoactivated localization microscopy (PALM), achieves imaging of the subcellular dynamics of PPIs at high spatial-temporal resolution (Liu et al., 2014).

Phosphorylation can be used to modulate PPIs, thereby regulating signaling transduction through either modifying proteins post-translationally or producing secondary messengers. Protein kinases play an important role in activating cellular processes, and the target proteins can be phosphorylated at different sites resulting in heterogeneous functions (Xue et al., 2012). Conventional methods for identifying kinase substrates suffer from several limitations. Kinase assay is one of the methods to identify kinase-substrate pairs by using analog-sensitive alleles. The entire in vitro substrate can be assayed for a particular kinase. Various synthesized analogs provided a panel for substrate identification and protein kinase function (Elphick et al., 2007). However, this method is not applicable when the introduced analog does not affect the kinase's function (Mok et al., 2009). Protein microarray analysis can also reveal the potential phosphorylation reactions as well as novel functions. To identify potential substrates, the protein kinases are added to arrays of immobilized proteins and generate signals upon detection (Meng et al., 2008). However, the relationships are identified in vitro and may not represent the relationships in vivo (Mok et al., 2009). Kinase substrate tracking and elucidation (KESTREL) has also been used to identify protein kinase substrates. The high background phosphorylation in the cell extracts can obscure the detection of putative phosphorylation, however, KESTREL achieves the identification of physiologically relevant substrates of proteins in crude cell extracts with an improved signal-to-noise ratio by limiting the phosphorylation reaction time and increasing concentrations of protein kinase of interest. Performing chromatography steps before the analysis could also greatly improve the SNR ratio. Also, to establish high physiological relevance, two or three closely related protein kinases with similar binding specificities were used for one substrate detection. However, the temporal and spatial information is largely ignored in the cell lysate (Knebel et al., 2001). However, the assays have experienced challenges with the background in cell extracts and medium-to-low specific activity to kinases (Knebel et al., 2001; Troiani et al., 2005).

High-throughput techniques such as mass cytometry have been widely used to identify the phosphorylation sites of the target protein. Although the bulk level correlation between the protein expression levels of markers was previously mapped out, the spatial correlations and the precise association between the kinases and their direct substrates are still unclear (Xue et al., 2012). Also, the cell-to-cell heterogeneity in the signaling transduction resulting from factors crucial for cancer development could not be systematically profiled due to technical limitations (Lun and Bodenmiller, 2020). This study could advance the current single-cell mass cytometry (CyTOF) in detecting kinases involved in signaling pathways using additional antibodies in the multiplexed imaging data (Han et al., 2015; Leelatian et al., 2015; Mingueneau et al., 2014). RapMIF provides spatial information on the protein distributions compared to traditional protein analysis assays. Since kinase assay requires the synthesis and introduction of ATP-analogs and is only applicable when the introduced analogs do not interfere with kinase’ function (Mok et al., 2009), RapMIF circumvents the complex process of synthesizing ATP-analogs by directly detecting protein activity from multiple cycles of IF. Also, unlike KESTREL, RapMIF conserves the subcellular structure by avoiding lysate in the cells.

The multiplexed single-pixel analyses have quantified the subcellular protein correlations, indicating potential direct physical neighborhood among signaling protein subsets. Distinct multiplexed intensity profiles of signaling markers revealed the crosstalk between heterogeneous signaling pathways. The presented RapMIF method simplified multiplexed protein analysis by pre-conjugating the antibodies with commercially available dyes. Also, it was observed that the bleaching solution interfered with Phalloidin signals in A549 cells. Therefore, in the A549 data analysis, a combination of p-EGFR, Concanavalin A, B-actin, WGA, APC, and WNT1 markers was used to outline the cell boundary for segmentation. Also, to avoid the bleaching effect, cycle 2 (Phalloidin and WGA) staining was done directly after cycle 1 (p-EGFR) without an intermediate bleaching step in PC9 multiplexing samples. A549 cells showed a larger cytosolic area than that of PC9 cells, providing a more detailed spatial network model for studying the protein distribution in the cytosol. The spatial maps demonstrated the translocation patterns of signaling markers corresponding to their functions in both cell types. The combination of ERK or MEK inhibition with Osimertinib could delay or overcome the acquired Osimertinib resistance (Gu et al., 2020a). Thus, the spatial signaling patterns in the MEK/ERK pathway, another downstream pathway of EGFR, regulating cell proliferation and survival could further be determined using additional antibody targets.

Even in the same population of cells, the cells may display heterogeneous signaling profiles due to the difference in signaling-acting pathways. Autocrine signaling involves the production and secretion of an extracellular mediator, acting on the same cell, while paracrine signaling occurs between different types of cells. Both signaling pathways mediate tumor growth, invasion, and metastasis. During cancer development, the growth factors are usually produced in an autocrine manner, allowing the cell to simulate itself in positive feedback (Caicedo, 2013; Ungefroren, 2021). Current methods have been developed to decipher the cell-cell communication in single cells, however, they failed to demonstrate the heterogeneity in autocrine and paracrine signaling interactions. CellChat as a predictive model for cell-cell communication has been achieved to study the autocrine-acting vs. paracrine-acting pathways in the same cell type or same subpopulation of cells. However, this approach highly relies on the accuracy of the ligand-receptor databases and it produces fewer interactions than other methods when detecting communications from distant cells (Jin et al., 2021). Therefore, combining predictive models and proteomics could optimize cell-cell communication methods. Also, the complex crosstalk among signaling pathways could contribute to the heterogeneity in signaling profiles. Fluorescent reporters can be introduced and utilized to track protein activity in the subpopulation of cells following drug treatment (Kurppa et al., 2020). However, the current study focuses on the design of signaling pathway analysis using molecular snapshots, instead of the subpopulation validation in real-time.

RapMIF multiplexed 17 signaling markers and converted their distributions into 33-pixel spatial clusters. Physical proximity analysis between two distinct proteins yielded a total of 272 (17 choose 2 combinations) possible pairwise neighborhoods, yielding their correlations in cytosolic and nuclear expression levels at the single-pixel level. Pairwise analysis of protein neighborhoods could reveal potential critical signaling proteins or networks (Ramana, 2019). Besides, coordination of the multi-protein neighborhood can be extracted from the multiplexed signaling data. For instance, the tri-protein interactions in cytosol and nucleus are assessed with 1360 (17 choose 3 combinations) possible three-way neighborhoods. Combinatorial signaling analysis would predict the dynamics of signaling networks when multiple proteins physically interact, a task that would be challenging to achieve using bulk signaling assays.

Our system design achieved a fully automated staining system including blocking, staining, imaging, and bleaching. The fluidic automation takes the antibody diluted in CSM media in the multi-well in a 2.5-fold dilution. Therefore, to save the antibody volume, the antibody loading step by step could be done by manually pipetting the antibodies to the sample. If needed, the automated system could be further optimized to reduce the dilution rate of the antibody during the staining step. Aspiration settings and washing speed could be adjusted to minimize cell loss across cycles. The p-EGFR signaling receptors in PC9 cells were dependent on the sample quality due to the step of drying the coverslip before mounting it to the holder. The freshness of the sample may affect the morphology of p-EGFR. Thus, the multiplexing experiments in PC9 cells were initiated on the same day of cell fixation to make sure the quality of the samples. Thus, the RapMIF could provide a precision oncology framework for defining the signaling dynamics in both intensities and spatial distribution of human cancers.

For multiplexing experiments, two factors are concerned to ensure cell quality, the efficiency of the bleaching approach and the total spent time for one set experiment. RapMIF utilizes fluorophore-conjugated antibodies for direct labeling. Fluorophore-conjugated antibodies generally produce relatively weaker signals than indirect labeling. However, indirect labeling raised some problems in the context of multiplexed experiments. It is constrained by the availability of the reactivity of secondary antibodies to limited species (e.g., mouse, human, donkey). Also, multiplexing indirect IF requires antibody stripping rather than a regular bleaching approach. The harsh process may cause cell loss and tissue detachment. Compared to the antibody stripping approach removing the whole secondary antibody, the RapMIF bleaching approach is effective in removing dyes while preserving the cells. One concern of the RapMIF is whether the direct IF staining at RT for 1-h for each cycle can label all the antigens in the cell. This trade-off can be addressed by improving the antibody concentration to generate similar signals in 1-h RT staining compared to overnight incubation. This approach aims to achieve rapid profiling of signaling pathways while maximizing the efficiency of protein detection. The staining settings were kept consistent across different samples, making it reliable for studying drug efficiency. Also, staining the sample for 1 hour instead of overnight reduces the 12-cycle multiplexing experiments from 24 days to 6 days, and prevents cell degradation. Another drawback of the RapMIF on FFPE tissues is that FFPE tissue generates a high autofluorescence background and is affected by shading or vignetting due to imaging artifacts. Signaling proteins are expected to express nearly everywhere, making it harder to distinguish between background and real signals. Therefore, background correction steps were performed using the BASIC algorithm to reduce the contribution of background on real signals (Peng et al., 2017). Also, post-bleach imaging data serve as a control of native autofluorescence signals and confirm the background corrected images using the stained raw tissue image that is subtracted from the post-bleach image.

In terms of antibody specificity validation, a positive control (e.g., a cell line expressing the target) and negative control (e.g., a cell line not expressing the target) can be utilized to confirm the selective binding of the antibody. Sometimes, it is difficult to find cell lines that do not express the target. Therefore, knock-out (KO) models such as CRISPR-Cas9 and siRNA can serve as negative controls, and be applied to various approaches, including mass spectrometry, western blot, IHC, and IF (Kurppa et al., 2020; Uhlen et al., 2016). In previous studies, independent antibody strategies are also suggested to confirm antibody specificity. When two antibodies bind to different epitopes of the same target, they can yield correlated signals, suggesting both antibodies recognize the same target (Uhlen et al., 2016). However, this is beyond the scope of current work and independent validations of antibody specificity are lacking in the current study. In this study, the antibody staining has been evaluated using conventional IF protocol before performing the multiplexing to improve the staining quality of the antibodies.

Another important consideration is the physical dimensions of signaling protein visualizations. The diffraction limit of light microscopy is around 200 nm, while the antibody size is around 10 nm (Erickson, 2009; Tan et al., 2008; Xing et al., 2016). Due to the low spatial resolution, standard light microscopy fails to resolve the protein complex interactions based on the fluorescent signals. To determine the colocalization of two proteins, their spatial distributions are needed. When two proteins are colocalized with one another, the overlay of the two spatial distributions should show a correlation of intensity at the pixel level. In this case, the pixel size is around 188 nm using an oil immersion 40× objective lens with a 1.4 numerical aperture and the protein molecule is around 2-9 nm (Erickson, 2009). For a single cell, the number of protein molecules is a median of 170,000 (Li et al., 2014) to 2×10⁹, considering the number of ribosomes, tRNA, synthesis rate, and translation rate, among others (Princiotta et al., 2003). Each pixel would contain around a few to 15,700 proteins. Thus, due to the limitation on the resolution of the microscope, it is unable to resolve the single protein interaction. Additional color deconvolution methods might be needed to reach single protein detection. The pixel-level correlations can distinguish the random color overlap due to protein compartmentalization from protein colocalization and indicate that true colocalization only occupied 3% of total colocalization (Costes et al., 2004). To remove nonspecific interactions in data analysis, a probabilistic protein interaction network has previously been developed to identify protein interactions based on the normalized spectral abundance factors using Bayes' approach. The probabilistic model predicts each pair of proteins by providing quantitative information on the preference of each interaction. It takes advantage of quantitative proteomics by measuring spectral counts to generate a probabilistic network of protein interactions, providing an alternative framework to quantify probabilistic pixel-level protein interactions in the RapMIF data (Sardiu et al., 2008). While it is not implemented, it can provide an alternative data analysis approach to quantify probabilistic pixel-level protein distributions in the same pixel area in the RapMIF data. Current studies focus on the development of the RapMIF pipelines for studying the crosstalk of multiple signaling pathways and the protein neighborhood likelihood using spatial networks by studying the neighboring protein distribution in the same pixel area. However, super-resolution can be implemented in this technology to further resolve protein complexes. Protein interaction detection assays, such as proximity ligation assays, can be multiplexed to achieve multiparameter detection of PPIs.

### Example 10—Multiplex PPI Detection Using RapMPI

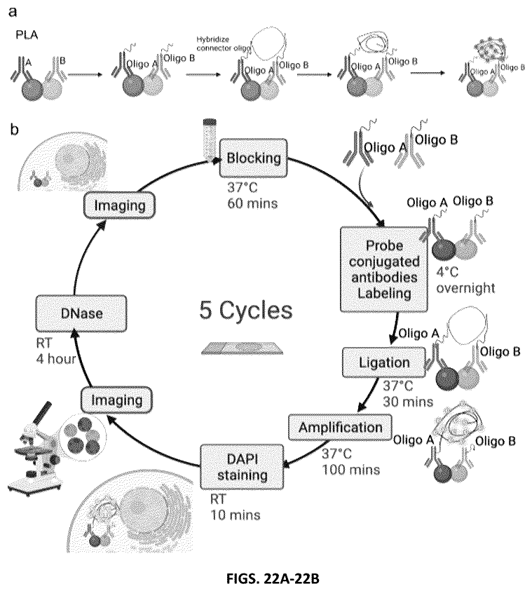

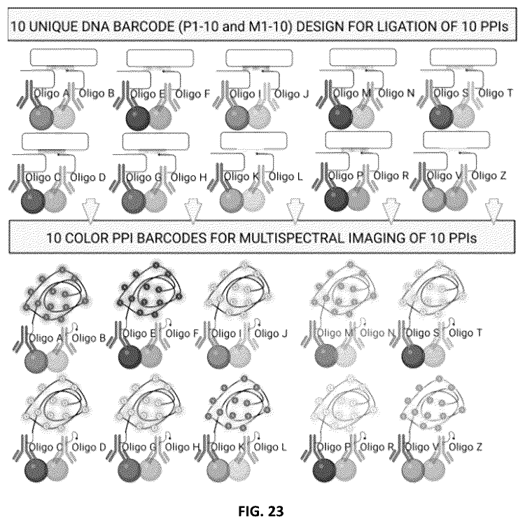

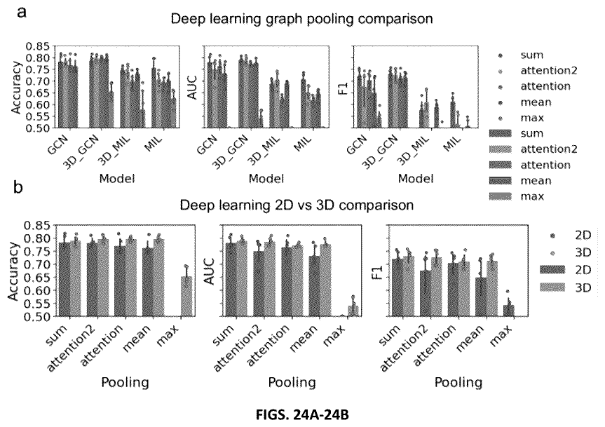

To detect complex PPI networks, a multiplexed PLA assay, RapMPI was developed that provides higher sensitivity and is compatible with both cells and tissues (frozen or FFPE). The detection can be either indirect or direct. For the detection of one PPI using direct labeling, the cells need to be stained with two primary antibodies targeting two different proteins or proteins phosphorylated at different sites^{17,18}. These primary antibody pairs are conjugated to one of the PLA probes, one to PLUS, and another one to MINUS (FIG. 22A). The PLA probe contains a unique oligonucleotide and is attached to the heavy chain of the primary antibody, and it permits the detection of PPIs in situ with a distance <40 nm at endogenous protein levels¹⁹. For indirect labeling where the oligonucleotides are conjugated to secondary antibodies. When the protein of interest interacts with each other, the DNA probes from two antibodies hybridize and ligate to form circular DNA¹⁹. Amplified circular DNA can be visualized using fluorescence microscopy. The commercial PLA detection kit allows visualization and quantification of the individual PPI. The current PLA technique was advanced to overcome the limitation on the number of protein pairs that can be detected by the conventional microscope. RapMPI can detect multiple PPIs by utilizing iterative cycles of labeling, imaging, DNasing, and re-labeling (FIG. 16A, FIG. 22B). The oligonucleotides on primary antibodies can be removed using DNase I. RapMPI utilizes commercial DuoLink PLA to detect highly multiplexed subcellular protein interaction maps. This iterative process can be repeated to create multiplexed signaling interaction maps in the same single cell. There are several ways to multiplex the PPI detection process. One pair of PPIs could be detected per cycle or three pairs of PPIs per cycle using multicolor detection. Also, by utilizing a multi-spectrum microscope, the detection of more than 3 pairs of PPIs per cycle could be achieved (FIG. 23).

RapMPI achieves profiling of multiple PPIs in the multiplexed single-cell and reconstructed their subcellular distributions. By incorporating the localization information of organelles and proliferation proteins using Rapid Multiplexed Immunofluorescence (RapMIF) (FIG. 16A)²⁰, RapMPI visualizes the protein associates across AKT/mTOR, MEK/ERK, and YAP/TEAD pathways at subcellular levels in NSCLC EGFRm cell cultures and EGFRm frozen mouse tissue samples. RapMPI experiments of cell cultures treated with Osimertinib demonstrate the PPIs dynamics and responses involved in the signaling pathways. SpPPi-GCN framework successfully predicts the cell perturbation states from the underlying PPI network graph created using PPI event distances. To validate the model, the spPPI-GCN model was compared with traditional machine learning (ML) models using PPI event counts, a multilayer perception (MLP) model using PPI counts, and a multi-instance learning (MIL) model using multilayer perception (FIG. 16B).

### Example 11—Highly Multiplex Spatial PPI Networks of PI3K/AKT/mTOR, MEK/ERK, and Hippo Pathways in EGFRm Cells

To reconstruct the signaling networks and PPI in EGFRm cells under drug perturbations, RapMPI was used to profile 5 PPIs involved in the AKT/mTOR, MEK/ERK, and YAP/TEAD pathways in NSCLC EGFRm Osimertinib-sensitive cell line, HCC827 cells. In a study, it was first investigated whether the EGFR pathways would be affected by Osimertinib in HCC827 cells, eventually affecting cell proliferation and growth. HCC827 cells were treated with and without 100 nM O for 6, 12, and 24 h. p-ERK was used to indicate the efficacity of Osimertinib treatment. P-ERK was suppressed initially after 6-hour treatment, however, Osimertinib even increased p-ERK after 12-hour treatment, suggesting Osimertinib may exert transient inhibitory effects on MEK/ERK pathway. To reveal the PPIs dynamics under Osimertinib treatment, RapMPI was performed on HCC827 cells treated without and with 100 nM Osimertinib for 12 h. To determine the best staining conditions for both RapMIF and RapMPI, titrations were performed on each antibody in the multiplexed panel. Immunofluorescence (IF) was performed to evaluate antibody staining using two different dilution rates. The SNRs of these IF images were compared, and the conditions with higher SNRs were used for either RapMPI or RapMIF experiments (TABLE 6). The staining dilutions were consistent with the manufacturer's suggested dilution range.

TABLE 6
SNR and dilution rates for signaling markers. The dilution rates
and corresponding SNR are displayed in the table. The values italicized
are the dilution rate used in the multiplex experiments.

| Marker | Dilution rate | SNR |
|---|---|---|
| CyclinD1 | 1:400 | 51.83062 |
| | 1:800 | 30.67618 |
| CDK4 | 1:200 | 65.32337 |
| | 1:400 | 29.55581 |
| Bim | 1:5000 | 31.10428 |
| | 1:6000 | 25.3655 |
| Sox2 | 1:100 | 103.459 |
| | 1:200 | 109.3032 |
| | 1:400 | 199.46 |
| Oct4 | 1:200 | 16.76949 |
| | 1:500 | 15.07917 |
| NFkb p65 | 1:800 | 41.04319 |
| | 1:1000 | 41.83676 |
| p-p90RSK | 1:800 | 28.82151 |
| | 1:1000 | 135.5758 |
| p-ERK | 1:100 | 20415.40 |
| | 1:200 | 45054.4 |
| YAP | 1:100 | 56040.8 |
| | 1:500 | 38597.6 |
| TEAD1 | 1:100 | 13573.20 |
| | 1:200 | 13969.8 |
| p-EGFR(y1068) | 1:100 | 14.59 |
| | 1:200 | 16.59 |
| mTOR | 1:200 | 12.52 |
| | 1:500 | 12.26729 |
| C-myc | 1:500 | 89.32363 |
| | 1:1000 | 90.24384 |
| BAK | 1:200 | 244.7008 |
| | 1:400 | 268.1081 |
| Mcl1 | 1:1600 | 37.94148 |
| | 1:4000 | 44.22946 |

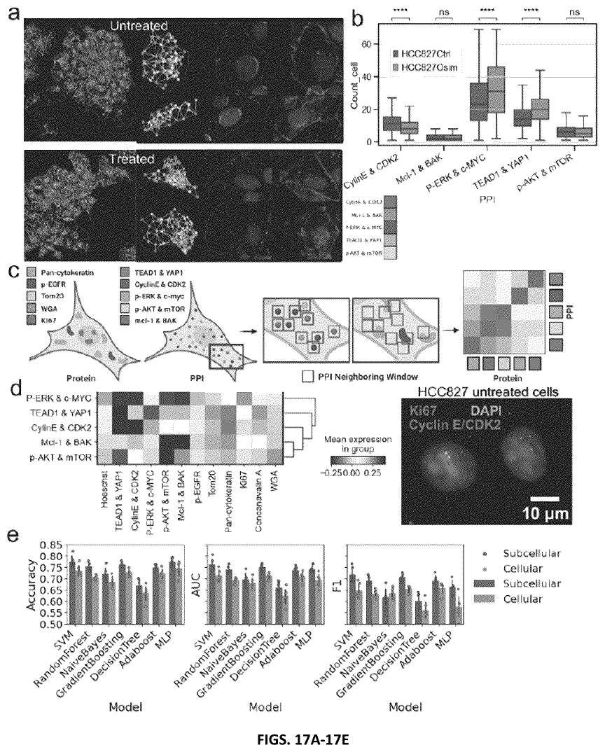

RapMPI demonstrated and compared the PPIs dynamics among proteins including YAP & TEAD1, Cyclin E & CDK2, p-ERK & c-MYC, Mcl-1 & Bak, and p-AKT & mTOR. The PPI distributions have been visualized using their spatial localization. Each node represents one detected PPI event, and for each single cell, a spatial graph of PPI events is constructed using Delaunay triangulation which captures the underlying spatial neighboring information of PPI events. The cell boundary was obtained from segmentation on the p-EGFR signaling protein (FIG. 17A).

The effect of Osimertinib on cell apoptosis and proliferation was first examined by comparing the Cyclin E/CDK2 and Mcl-1/Bak interactions. MCL-1 is known as an anti-apoptotic factor, and it can be phosphorylated by ERK, resulting in enhanced proteasome-dependent degradation. Osimertinib downregulates Mcl-1, therefore, enhancing cell apoptosis²¹. Mcl-1 can sequester Bak activity via direct interaction, therefore preventing cell apoptosis²². The interaction between Mcl-1 and Bak is related to the inhibition of cell apoptosis. After Osimertinib treatment, the cells did not exhibit an obvious change in the Mcl1/Bak interaction in either the cytoplasm or the whole cell (FIG. 17B). However, Osimertinib effectively inhibited the interaction between cyclin E and CDK2 (FIG. 17B), which functions in initiating the S phase and cell proliferation²³.

The effects of Osimertinib on the EGFR-related signaling pathways were next determined. Osimertinib as an inhibitor to EGFR downregulated the p-AKT/mTOR pathway reducing the interactions in the cytoplasm. Osimertinib can induce the degradation of c-MYC, which regulates cell growth and proliferation in EGFRm-sensitive cells, and ERK can phosphorylate c-Myc at S62 linked to c-MYC's stabilization²⁴. However, the inhibitory effect is limited in Osimertinib-resistant cell lines, and the upregulation of c-Myc is related to the acquired resistance to Osimertinib²⁴. In terms of the YAP/TEAD1 pathway, YAP can remain active under EGFR TKIs and EGFR/MEK inhibitions contributing to the tumor dormancy in EGFRm NSCLC4. It was observed that after treatment the cells exhibited significantly increased interactions of YAP/TEAD1 and P-ERK/c-Myc (FIG. 17B), indicating that Osimertinib may change the signaling dynamics by upregulating YAP and ERK pathways as a compensatory to EGFR inhibition. Previous studies examining the effect of Osimertinib on p-ERK using western blot demonstrated the inhibitory role of Osimertinib on p-ERK for up to 24 hours using western blot1. However, western blot measures the total protein level, which is different from the protein interactions where proteins function in active forms.

To resolve the spatial proteomics and PPIs, RapMIF was also into the panel²⁰. RapMIF was utilized to detect the localization of distinct organelles (e.g., Golgi: WGA, Endoplasmic reticulum: concanavalin A, Mitochondria: TOM20, nucleus: DAPI). The organelle information can be associated with the distribution of PPIs from RapMPI to identify the subcellular localization of PPIs (receptors, cytosol, or nucleus) in individual cells (FIG. 17C). The PPI co-expression confirmed the localization of Mcl-1/Bak interactions in mitochondria with high coexpression of TOM20 (FIG. 17D). The co-expression of Cyclin E/CDK2 and ki67 in the nucleus was also observed (FIG. 17D). All three markers are related to cellular proliferation, and they are highly correlated with each other in colorectal carcinoma²⁵. Also, RapMPI revealed the co-localization of MCL-1/Bak and p-AKT/mTOR PPIs (FIG. 17D). That is potentially due to the regulation of the AKT pathway in mitochondria-mediated functions such as redox states, apoptosis, and metabolism²⁶.

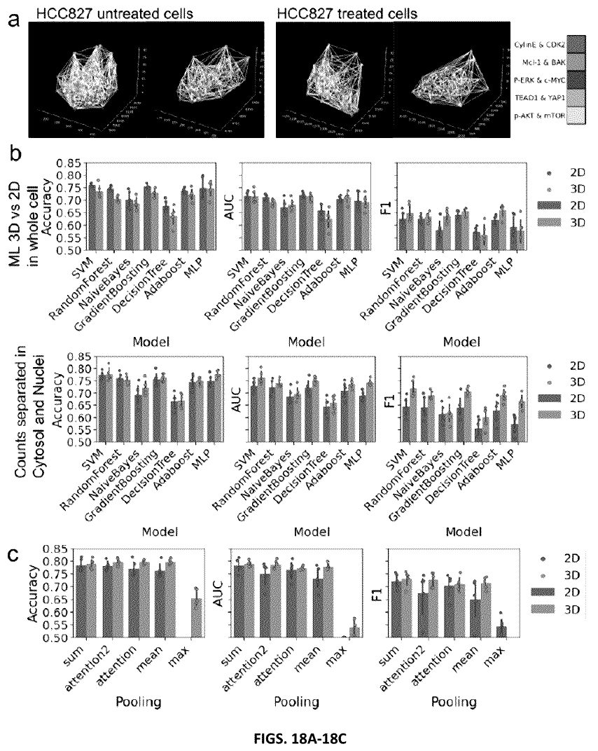

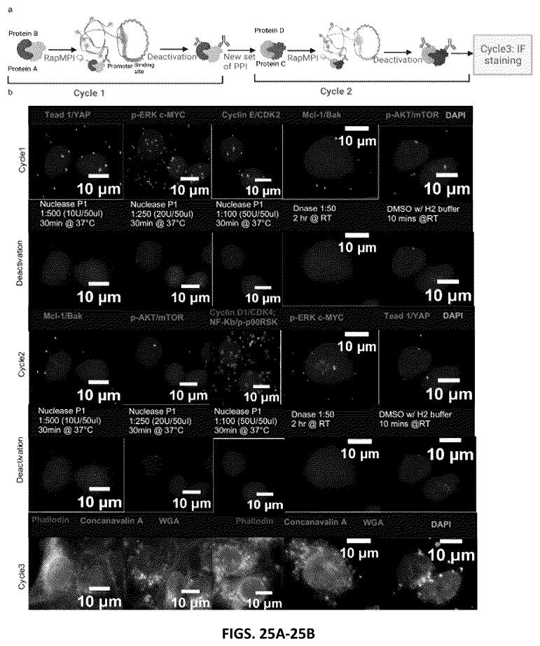

PPI events distribution as predictive features to respond to drug treatment was investigated by training models to predict the treatment output based on the PPI event counts. First, six machine learning models and one MLP model were trained to evaluate the accuracy, Area Under the Curve (AUC), and F1 scores to compare subcellular feature importance. The MLP model consists of stacked fully connected layers with input the same as the machine learning models. The separation of PPI events into cytoplasmic and nucleic compartments showed improvement in all metrics across models (FIG. 17E, TABLE 7). Next, the importance of 3D information was explored by comparing the PPI count obtained from maximum projection images (2D) or individual z-stack images (3D). 3D PPI events information gives better prediction compared to 2D in both whole cell and subcellular (FIGS. 18A-18B, TABLES 7-8). MLP model performance is comparable to machine learning models in 3D while worse in 2D. On the other hand, the MLP model seems to have similar performance in subcellular settings showing good generalizability of deep learning models. Finally, deep-learning models for prediction were compared by using MIL and spPPI-GCN models (FIG. 18C, FIGS. 24A-24B, TABLE 9). The MIL model is similar to the MLP model but instead of PPI event counts as input a matrix concatenating the one-hot encoding of the PPI events for each cell was fed and a pooling layer was introduced. The spPPI-GCN model works in the same setting as MIL, but a spatial graph of PPI events was input, and therefore spatial neighboring information was introduced to the model. It was showed that the spPPI-GCN model performed better in 2D, and 3D compared to all other benchmark models (TABLE 10). In the spPPI-GCN and MIL model, different graph pooling layers were assessed to see how different feature embedding aggregations affect the model (FIGS. 24A-24B).

TABLE 7
Machine learning model prediction of single-cell drug treatment
perturbation from 2D maximum projection for 5 PPI dataset.

| Model | Type | Accuracy | AUC | F1 |
|---|---|---|---|---|
| Adaboost | Cellular | 0.738 +− 0.01 | 0.705 +− 0.012 | 0.62 +− 0.02 |
| | Subcellular | 0.744 +− 0.032 | 0.711 +− 0.036 | 0.628 +− 0.054 |
| DecisionTree | Cellular | 0.676 +− 0.023 | 0.658 +− 0.026 | 0.573 +− 0.037 |
| | Subcellular | 0.665 +− 0.036 | 0.645 +− 0.039 | 0.556 +− 0.047 |
| GradientBoosting | Cellular | 0.755 +− 0.009 | 0.721 +− 0.011 | 0.641 +− 0.018 |
| | Subcellular | 0.756 +− 0.029 | 0.722 +− 0.032 | 0.641 +− 0.047 |
| MLP | Cellular | 0.748 +− 0.045 | 0.696 +− 0.037 | 0.592 +− 0.052 |
| | Subcellular | 0.748 +− 0.025 | 0.689 +− 0.02 | 0.574 +− 0.041 |
| NaiveBayes | Cellular | 0.702 +− 0.037 | 0.671 +− 0.031 | 0.579 +− 0.043 |
| | Subcellular | 0.692 +− 0.045 | 0.686 +− 0.032 | 0.615 +− 0.039 |
| RandomForest | Cellular | 0.746 +− 0.011 | 0.71 +− 0.012 | 0.626 +− 0.018 |
| | Subcellular | 0.761 +− 0.022 | 0.724 +− 0.03 | 0.643 +− 0.048 |
| SVM | Cellular | 0.761 +− 0.009 | 0.716 +− 0.016 | 0.626 +− 0.03 |
| | Subcellular | 0.772 +− 0.016 | 0.728 +− 0.024 | 0.645 +− 0.04 |

TABLE 8
Machine learning model prediction of single-cell drug treatment
perturbation from 3D z-stacks for 5 PPI dataset.

| Model | Type | Accuracy | AUC | F1 |
|---|---|---|---|---|
| Adaboost | Cellular | 0.722 +− 0.024 | 0.71 +− 0.023 | 0.659 +− 0.028 |
| | Subcellular | 0.75 +− 0.015 | 0.738 +− 0.019 | 0.691 +− 0.028 |
| DecisionTree | Cellular | 0.637 +− 0.038 | 0.625 +− 0.04 | 0.56 +− 0.049 |
| | Subcellular | 0.671 +− 0.029 | 0.66 +− 0.033 | 0.602 +− 0.044 |
| GradientBoosting | Cellular | 0.729 +− 0.017 | 0.713 +− 0.015 | 0.656 +− 0.017 |
| | Subcellular | 0.763 +− 0.016 | 0.751 +− 0.015 | 0.707 +− 0.017 |
| MLP | Cellular | 0.747 +− 0.034 | 0.692 +− 0.033 | 0.577 +− 0.058 |
| | Subcellular | 0.776 +− 0.016 | 0.742 +− 0.014 | 0.665 +− 0.029 |
| NaiveBayes | Cellular | 0.687 +− 0.024 | 0.681 +− 0.024 | 0.636 +− 0.026 |
| | Subcellular | 0.721 +− 0.031 | 0.696 +− 0.03 | 0.618 +− 0.043 |
| RandomForest | Cellular | 0.706 +− 0.011 | 0.691 +− 0.012 | 0.633 +− 0.02 |
| | Subcellular | 0.754 +− 0.018 | 0.74 +− 0.017 | 0.691 +− 0.018 |
| SVM | Cellular | 0.736 +− 0.027 | 0.714 +− 0.027 | 0.649 +− 0.036 |
| | Subcellular | 0.776 +− 0.028 | 0.763 +− 0.029 | 0.719 +− 0.036 |

TABLE 9
Deep learning model prediction of single-cell drug treatment perturbation
from 2D maximum projection and 3D z-stacks for 5 PPI dataset.

| Model | Pooling | Accuracy | AUC | F1 |
|---|---|---|---|---|
| scPPI-GNN 3D | attention | 0.796 +− 0.009 | 0.772 +− 0.008 | 0.71 +− 0.03 |
| | attention2 | 0.796 +− 0.015 | 0.786 +− 0.017 | 0.726 +− 0.03 |
| | max | 0.655 +− 0.035 | 0.541 +− 0.035 | 0.204 +− 0.167 |
| | mean | 0.796 +− 0.011 | 0.776 +− 0.012 | 0.714 +− 0.024 |
| | sum | 0.788 +− 0.02 | 0.789 +− 0.012 | 0.731 +− 0.031 |
| MIL 3D | attention | 0.698 +− 0.037 | 0.62 +− 0.02 | 0.437 +− 0.048 |
| | attention2 | 0.739 +− 0.034 | 0.704 +− 0.035 | 0.61 +− 0.054 |
| | max | 0.577 +− 0.131 | 0.5 +− 0.0 | 0.105 +− 0.235 |
| | mean | 0.73 +− 0.02 | 0.688 +− 0.013 | 0.588 +− 0.03 |
| | sum | 0.746 +− 0.013 | 0.689 +− 0.02 | 0.575 +− 0.046 |
| scPPI-GNN | attention | 0.769 +− 0.032 | 0.764 +− 0.037 | 0.703 +− 0.047 |
| | attention2 | 0.78 +− 0.019 | 0.749 +− 0.047 | 0.675 +− 0.09 |
| | max | 0.374 +− 0.034 | 0.5 +− 0.0 | 0.544 +− 0.036 |
| | mean | 0.762 +− 0.03 | 0.732 +− 0.047 | 0.649 +− 0.086 |
| | sum | 0.783 +− 0.026 | 0.78 +− 0.028 | 0.721 +− 0.029 |
| MIL | attention | 0.693 +− 0.023 | 0.618 +− 0.019 | 0.437 +− 0.037 |
| | attention2 | 0.704 +− 0.036 | 0.65 +− 0.033 | 0.516 +− 0.049 |
| | max | 0.626 +− 0.034 | 0.5 +− 0.0 | 0.0 +− 0.0 |
| | mean | 0.701 +− 0.033 | 0.644 +− 0.024 | 0.508 +− 0.032 |
| | sum | 0.756 +− 0.038 | 0.706 +− 0.027 | 0.612 +− 0.036 |

TABLE 10
Maximum metrics for each category of machine learning,
MLP, MIL, and GNN models for 5 PPI dataset.

| Model | Type | Accuracy | AUC | F1 | Dimension |
|---|---|---|---|---|---|
| SVM | Cellular | 0.768 | 0.737 | 0.667 | 2D |
| MLP | Cellular | 0.748 | 0.696 | 0.592 | |
| GradientBoosting | Subcellular | 0.801 | 0.773 | 0.717 | |
| MLP | Subcellular | 0.748 | 0.689 | 0.574 | |
| GCN | sum | 0.806 | 0.813 | 0.745 | |
| MLP | sum | 0.796 | 0.736 | 0.648 | |
| SVM | Cellular | 0.779 | 0.753 | 0.694 | 3D |
| MLP | Cellular | 0.747 | 0.692 | 0.577 | |
| SVM | Subcellular | 0.821 | 0.806 | 0.766 | |
| MLP | Subcellular | 0.776 | 0.742 | 0.665 | |
| GCN | attention2 | 0.812 | 0.809 | 0.752 | |
| MLP | attention2 | 0.771 | 0.742 | 0.667 | |

### Example 12—Scaling Up the Profiling of Signaling Networks in EGFRm Cells

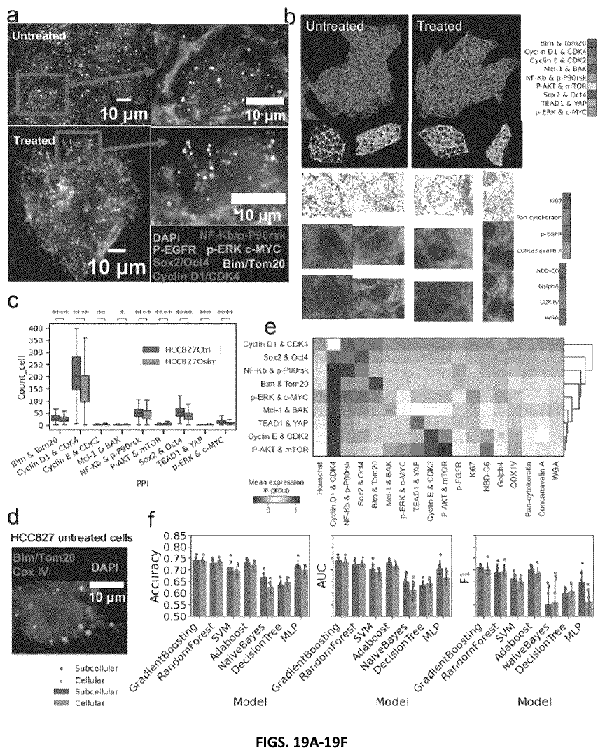

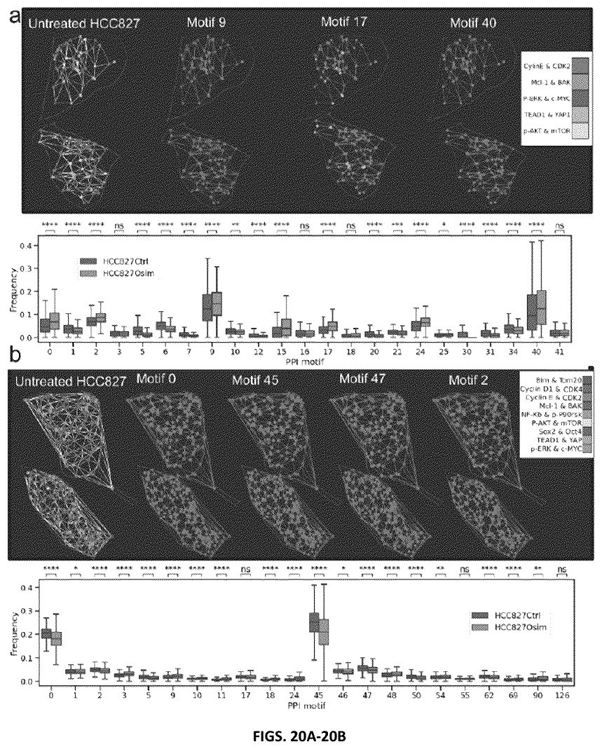

To confirm the feasibility of scaling up the PPI detection, 9 PPIs were profiled in HCC827 cells treated with and without Osimertinib for 12 hours. The cells were performed with two cycles of multicolor detection followed by five cycles of single detection of PPIs (YAP/TEAD1, Cyclin E/CDK2, p-ERK/c-Myc, p-AKT/mTOR, Mcl-1/Bak). The multicolor detection allows detection of 2 to 3 pairs of PPIs in a single cycle. Followed by RapMPI, RapMIF was performed to detect organelle locations, cell proliferation, and tumor cells. The PPI distribution in every single cell can be visualized using the spatial network (FIGS. 19A-19B).

In addition to cyclin E/CDK2, cyclin D1/CDK4 was used to detect the GUS transition in the cell cycle²⁷. Osimertinib exhibited an inhibitory effect on cyclin D1/CDK4 (FIG. 19C). However, similar upregulation patterns of p-ERK/c-Myc were not observed after treatment in 9 PPI data (FIG. 19C) compared to 5 PPI data. That is probably due to the subpopulation heterogeneity among cells, and p-ERK/c-Myc exhibited a relatively higher expression level in the 5PPI dataset from the UMAP highlighted in red. The interaction between p-p90RSK and NF-κB p65 was also examined. P-p90RSK as an ERK substrate phosphorylates p65 at S276 in an ERK-dependent manner, leading to inflammation response²⁸. Osimertinib effectively inhibited the PPIs of NF-κB/p-p90RSK and p-ERK/c-Myc, suggesting the downregulation of the MEK/ERK pathway followed by Osimertinib treatment. Sox2 as a transcription factor can incorporate with Oct4 to maintain stem-like properties. The downregulation of sox2/oct4 demonstrates the inhibitory effect of Osimertinib in EGFRm-sensitive cells (FIG. 19C). However, it has been found that Osimertinib-resistant EGFRm NSCLC cell lines express high levels of sox2 and increased autophagy^{29,30}. To evaluate the cell apoptosis, in addition to Mcl-1/Bak, the interaction between Tom20 and Bim was also profiled. Bim as a pro-apoptotic Bcl-2-family protein can interact with Tom20 independent of the binding to anti-apoptotic proteins³¹. Tom20 protein is inserted in the outer mitochondria membrane (OMM) and may function in the regulation of Bim-localization into mitochondria. Reduced interactions between Tom20 and Bim were observed after 12-hour Osimertinib. However, Bim may translocate into OMM without TOM receptors, and Tom20 also mediates the transfer of antiapoptotic Bcl2 proteins into mitochondria^{31,32}. More studies are needed to conclude if Osimertinib enhances cell apoptosis in HCC827 cells. From the image, Cox IV, a mitochondria marker colocalized with Bim/Tom20 PPI (FIG. 19D), was observed, confirming the location of Bim/Tom20 interaction in mitochondria. Due to the ununiform signals of Cox IV in large ROI, the Cox IV positive regions were filtered out for co-expression and correlation analysis. No colocalization was observed between Bim/Tom20 and Cox IV (FIG. 19E). That's potentially due to the large-scale normalization overlooking the co-expression variety. The co-expression analysis was performed between 5 relatively highly expressed PPIs and 8 protein markers and confirmed the colocalization between Bim/Tom20 and Cox IV.

Cyclin E/CDK2 colocalizes in the Golgi with high co-expression of NBD-C6 (FIG. 19E). Cyclin E regulates the transition of the G1 to S phase in the cell cycle. The activity of Cyclin E can be directly regulated by RhoBTB3, a Golgi-localized and -associated protein. The direct interaction between cyclin E and RhoBTB3 mediates the ubiquitylation and turnover of cyclin E during the S phase³³. Also, the co-expression between p-AKT/mTOR and NBD-C6 or Golph4 in cytosol demonstrated the potential regulation of Golgi on mTOR signaling. Golgi can modulate mTOR activity in several ways including downregulating autophagy by activating mTOR, and Golph2, a Golgi protein, has been found to promote mTOR activity through the PI3K/AKT pathway³⁴.

It was observed that the later cycles of RapMPI such as TEAD1/YAP and Cyclin E/CDK4 exhibited much fewer signals. That may be because of Dnase digesting DNA-connected proteins in the nucleus, especially transcription factors. To minimize the Dnase effect on nuclear proteins, the order of PPI detections was changed, and TEAD1/YAP and Cyclin E/CDK4 PPIs were not affected in the first two cycles. It was observed that multicolor detection was less sensitive to DNase and bleaching, and the residuals of PPI could be re-detected after 6 times DNase and bleaching.

To preserve the quality of single-color PPI detection, nuclease P1 as an alternative reagent to DNase to digest single-strand DNA was tested on five pairs of PPIs using single-color detection in HCC827 untreated cells (FIGS. 25A-25B). Nuclease P1 concentrations ranging from 1:500 to 1:100 worked effectively in removing single-stranded DNAs bound to fluorophores. In addition to nuclease P1, it was also demonstrated that shortening the DNase incubation from 4 hours to 2 hours can still remove the oligos and fluorophores. DMSO stripping solution exhibited practicability in deactivating the signals³⁵. By re-staining the samples with another set of PPIs, the feasibility of multiplexing PPIs using nuclease P1 and DMSO stripping approaches was confirmed. It was also observed that nuclease P1 has a lower effect on phalloidin by preserving its phenotype compared to DNase and DMSO.

Similarly, machine learning (FIG. 19F) and deep learning pipelines (TABLES 11-13) were compared for the prediction of drug perturbation. Using the same models and settings as previously described, prediction metrics were obtained using PPI events spatial graphs. The overall prediction results showed higher Accuracy, AUC, and F1 when using spPPIGCN compared to other benchmark models (TABLE 14).

TABLE 11
Machine learning model prediction of single-cell drug treatment
perturbation from 2D maximum projection for 9 PPI dataset.

| Model | Type | Accuracy | AUC | F1 |
|---|---|---|---|---|
| Adaboost | Cellular | 0.72 +− 0.017 | 0.717 +− 0.016 | 0.685 +− 0.029 |
| | Subcellular | 0.733 +− 0.013 | 0.731 +− 0.014 | 0.703 +− 0.017 |
| DecisionTree | Cellular | 0.647 +− 0.023 | 0.644 +− 0.024 | 0.608 +− 0.035 |
| | Subcellular | 0.638 +− 0.019 | 0.636 +− 0.02 | 0.601 +− 0.02 |
| GradientBoosting | Cellular | 0.738 +− 0.018 | 0.735 +− 0.02 | 0.703 +− 0.032 |
| | Subcellular | 0.744 +− 0.016 | 0.742 +− 0.017 | 0.713 +− 0.012 |
| MLP | Cellular | 0.7 +− 0.027 | 0.668 +− 0.037 | 0.563 +− 0.082 |
| | Subcellular | 0.719 +− 0.029 | 0.706 +− 0.041 | 0.646 +− 0.067 |
| NaiveBayes | Cellular | 0.626 +− 0.034 | 0.613 +− 0.053 | 0.564 +− 0.21 |
| | Subcellular | 0.669 +− 0.025 | 0.648 +− 0.053 | 0.553 +− 0.162 |
| RandomForest | Cellular | 0.732 +− 0.017 | 0.729 +− 0.017 | 0.696 +− 0.027 |
| | Subcellular | 0.731 +− 0.019 | 0.726 +− 0.024 | 0.691 +− 0.044 |
| SVM | Cellular | 0.697 +− 0.025 | 0.689 +− 0.021 | 0.648 +− 0.026 |
| | Subcellular | 0.712 +− 0.036 | 0.705 +− 0.032 | 0.664 +− 0.026 |

TABLE 12
Machine learning model prediction of single-cell drug treatment
perturbation from 3D z-stacks for 9 PPI dataset.

| Model | Type | Accuracy | AUC | F1 |
|---|---|---|---|---|
| Adaboost | Cellular | 0.713 +− 0.03 | 0.685 +− 0.022 | 0.6 +− 0.017 |
| | Subcellular | 0.719 +− 0.033 | 0.691 +− 0.03 | 0.605 +− 0.035 |
| DecisionTree | Cellular | 0.64 +− 0.021 | 0.625 +− 0.028 | 0.544 +− 0.045 |
| | Subcellular | 0.614 +− 0.016 | 0.593 +− 0.018 | 0.493 +− 0.03 |
| GradientBoosting | Cellular | 0.722 +− 0.023 | 0.689 +− 0.016 | 0.601 +− 0.022 |
| | Subcellular | 0.746 +− 0.029 | 0.718 +− 0.033 | 0.639 +− 0.05 |
| MLP | Cellular | 0.7 +− 0.027 | 0.668 +− 0.037 | 0.563 +− 0.082 |
| | Subcellular | 0.719 +− 0.029 | 0.706 +− 0.041 | 0.646 +− 0.067 |
| NaiveBayes | Cellular | 0.633 +− 0.034 | 0.544 +− 0.032 | 0.238 +− 0.089 |
| | Subcellular | 0.621 +− 0.032 | 0.531 +− 0.015 | 0.207 +− 0.035 |
| RandomForest | Cellular | 0.706 +− 0.027 | 0.674 +− 0.025 | 0.58 +− 0.037 |
| | Subcellular | 0.741 +− 0.033 | 0.709 +− 0.032 | 0.627 +− 0.046 |
| SVM | Cellular | 0.689 +− 0.039 | 0.632 +− 0.03 | 0.474 +− 0.065 |
| | Subcellular | 0.731 +− 0.036 | 0.687 +− 0.029 | 0.581 +− 0.039 |

TABLE 13
Deep learning model prediction of single-cell
drug treatment perturbation from 2D maximum
projection and 3D z-stacks for 9 PPI dataset.

| Model | Pooling | Accuracy | AUC | F1 |
|---|---|---|---|---|
| GCN 3D | attention | 0.796 +− 0.021 | 0.786 +− 0.031 | 0.743 +− 0.046 |
| | attention2 | 0.787 +− 0.018 | 0.774 +− 0.018 | 0.728 +− 0.034 |
| | max | 0.667 +− 0.031 | 0.6 +− 0.027 | 0.366 +− 0.095 |
| | mean | 0.783 +− 0.017 | 0.759 +− 0.024 | 0.701 +− 0.04 |
| | sum | 0.794 +− 0.013 | 0.776 +− 0.022 | 0.725 +− 0.034 |
| MIL 3D | attention | 0.7 +− 0.012 | 0.653 +− 0.022 | 0.512 +− 0.049 |
| | attention2 | 0.722 +− 0.017 | 0.696 +− 0.018 | 0.62 +− 0.029 |
| | max | 0.441 +− 0.09 | 0.524 +− 0.054 | 0.581 +− 0.032 |
| | mean | 0.722 +− 0.011 | 0.685 +− 0.013 | 0.581 +− 0.032 |
| | sum | 0.723 +− 0.021 | 0.693 +− 0.02 | 0.612 +− 0.037 |
| GCN | attention | 0.742 +− 0.048 | 0.716 +− 0.06 | 0.639 +− 0.1 |
| | attention2 | 0.748 +− 0.036 | 0.732 +− 0.039 | 0.68 +− 0.072 |
| | max | 0.699 +− 0.026 | 0.661 +− 0.019 | 0.539 +− 0.028 |
| | mean | 0.75 +− 0.031 | 0.744 +− 0.032 | 0.698 +− 0.058 |
| | sum | 0.729 +− 0.07 | 0.729 +− 0.034 | 0.689 +− 0.017 |
| MIL | attention | 0.711 +− 0.01 | 0.685 +− 0.021 | 0.604 +− 0.051 |
| | attention2 | 0.701 +− 0.017 | 0.668 +− 0.023 | 0.566 +− 0.046 |
| | mean | 0.709 +− 0.023 | 0.68 +− 0.021 | 0.59 +− 0.027 |
| | sum | 0.71 +− 0.02 | 0.674 +− 0.035 | 0.565 +− 0.079 |
| | max | 0.417 +− 0.014 | 0.5 +− 0.0 | 0.588 +− 0.014 |

TABLE 14
Maximum metric for each category of machine learning,
MLP, MIL, and GNN models for 9 PPI dataset.

| Model | Type | Accuracy | AUC | F1 | 2D |
|---|---|---|---|---|---|
| GradientBoosting | Cellular | 0.7660 | 0.7655 | 0.7559 | 2D |
| MLP | Cellular | 0.7003 | 0.6677 | 0.5634 | |
| MLP | Subcellular | 0.7628 | 0.7661 | 0.7280 | |
| MLP | Subcellular | 0.7186 | 0.7061 | 0.6462 | |
| GCN | attention2 | 0.7949 | 0.7861 | 0.7500 | |
| MLP | sum | 0.7399 | 0.7197 | 0.6570 | |
| Adaboost | Cellular | 0.7456 | 0.7162 | 0.6282 | 3D |
| MLP | Cellular | 0.7003 | 0.6677 | 0.5634 | |
| MLP | Subcellular | 0.7628 | 0.7661 | 0.7280 | |
| MLP | Subcellular | 0.7186 | 0.7061 | 0.6462 | |
| GCN | attention | 0.8108 | 0.8111 | 0.7803 | |
| MLP | attention2 | 0.7413 | 0.7229 | 0.6633 | |

The small groups of interconnections among PPIs were also visualized using network motifs. These network motifs are statistically significant patterns within large spatial signaling networks. Increased autoregulation of p-ERK/c-Myc PPI activity was observed after Osimertinib treatment in motif 9 (p-ERK/c-Myc & p-ERK/c-Myc), and the PPI of p-ERK/c-Myc occupies a large proportion in the network (FIG. 20A), which suggests p-ERK/c-Myc may be the main effector of Osimertinib. Also, more interactions between p-ERK/c-Myc and Tead1/Yap were observed after treatment in a three-node motif 17 (Tead1/Yap&Tead1/Yap& p-ERK/c-Myc). That indicates Osimertinib may potentially increase the crosstalk between ERK and TEAD pathways. It has been found that with the combination of ERK and Osimertinib treatment, the cells can still survive with upregulated YAP activity4. In the 9 PPIs dataset, the autoregulation of cyclin D1/CDK2 in motif 0 and motif 45 was inhibited by Osimertinib treatment (FIG. 20B), indicating suppressed cell cycle progression. Also, downregulation of the interaction between sox2/oct4 and Cyclin D1/CDK4 was observed in both two-node (motif 2) and three-node interaction motifs (motif 47). Sox2 is highly expressed while repressing the expression of Cyclin D1 in stem cells³⁶ The reduced interactions may indicate the potential role of Osimertinib in regulating stem-cell-like properties and cell proliferation. Dissecting the spatial networks into subgraph motifs reveals the organization of signaling networks. These small functional building blocks reflect the interactions between PPIs are not random, which provides important clues to identify potential biomarkers for targeting signaling crosstalk.

### Example 13—Spatial Signaling PPI Networks in Lung Tissues

To verify the feasibility of detecting PPIs in situ, RapMPI was investigated on HCC827-derived mouse xenografts (CDX). The RapMPI on cell culture resolves the PPI at the subcellular level, however, it fails to consider the architecture of cellular distributions. A study utilized RapMPI on tissue achieving to generate single-cell signaling maps within the context of the tumor microenvironment.

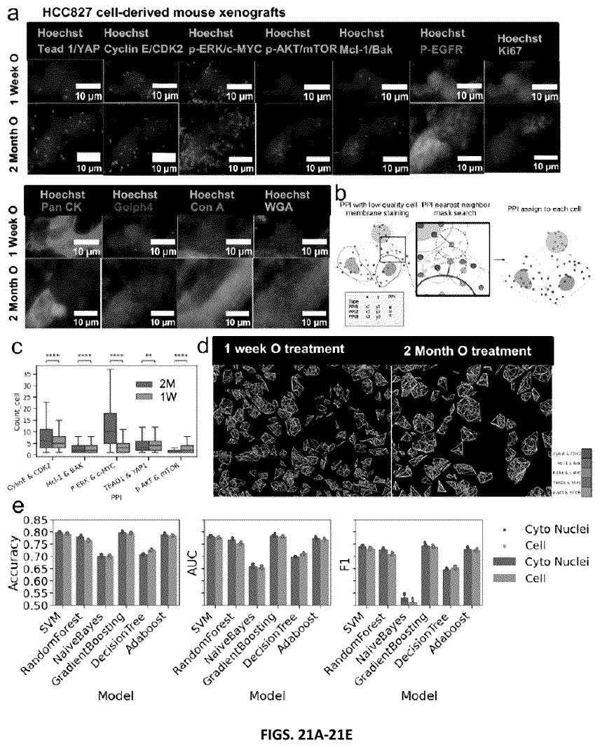

HCC827-derived xenografts in mice were treated with and without Osimertinib for a sustained period, and OCT sections were obtained to compare the emergence of resistance. Osimertinib was given to mice daily. HCC827 xenografts in mice receiving Osimertinib treatment were effectively inhibited for the first week. The tumor almost disappeared after a 5-day Osimertinib treatment. As treatment continued, the tumor grew back and larger, indicating the development of acquired resistance to Osimertinib. Therefore, in the study, RapMPI was performed on mouse HCC827 cells-derived xenografts tissues in the presence of Osimertinib treatment for 1 week and 2 months, representing responders and non-responders. The dynamics of 5 PPIs related to organelle localization, proliferation markers, and gene expression were detected (FIG. 21A). Pan-cytokeratin was utilized to help differentiate tumor regions. The nearest-pixel method was utilized to assign PPI signals to the nearest cell with incomplete cell segmented regions (FIG. 21B). It was determined that Osimertinib enhanced MEK/ERK signaling while downregulating AKT/mTOR pathways after 2-month treatment (FIG. 21C). That indicates mice became less sensitive to Osimertinib and even developed acquired resistance to Osimertinib functioning as non-responders after 2-month treatment (FIG. 21C). Also, mouse tissues treated with 2-month Osimertinib expressed more pancytokeratin-positive cells. The downregulation of cyclin E/CDK2 in responders also indicates inhibited cell cycle progression (FIG. 21C).

A spatial network has been used to visualize the PPI distributions at the single-cell level in the tissues (FIG. 21D). The predictive models exhibited similar performance between whole cell and subcellular regions (FIG. 21E). The architecture of the dense-packed cells in tissues may minimize the difference in PPI distribution in cytosol and nuclei while single cell segmentation might affect accurate subcellular compartment detection.

To demonstrate the feasibility of detecting PPIs using a super-resolved microscope, the RapMPI detected by a widefield microscope and Zeiss 900A with Airy scan was compared. The negative control with one MINUS probe alone confirmed the validity of the RapMPI protocol.

Discussion: RapMPI is an image-based multiplexing approach to detect PPI at the subcellular level. The iterative processes of ligation, amplification, imaging, and DNasing allows detection of 9 PPIs in cell cultures and 5 PPIs in tissues. The feasibility of integrating RapMPI with RapMIF to profile both PPIs and signaling, proliferation, and organelles' markers is demonstrated herein. The upregulation of Tead1/YAP and p-ERK/c-Myc PPIs after Osimertinib treatment in HCC827 cells may indicate the activation of YAP and the p-ERK pathway as compensatory pathways to EGFR inhibition. The co-expression analysis evaluated the co-localization of signaling markers with organelles. Also, it was observed that Dnase interfered with Phalloidin signaling in HCC827 cells. Therefore, a combination of p-EGFR, concanavalin, and WGA was used for cell segmentation. DNase digests both single-strand and double-strand DNA in the nucleus, suggesting that it may change the localization of proteins connected with DNA and reduce PPI detection in later cycles. To reduce the negative effect of Dnase on PPI detection, further experiments were conducted on examining the effects of nuclease P1 and DMSO stripping solution on deactivating oligos and fluorophores. Nuclease P1 and DMSO exhibited comparable effects on digesting DNA as Dnase. Nuclease P1 targeting only single-strand DNA serves as a good alternative to nuclease P1. Also, it preserves the decent staining of Phalloidin, indicating that it has minimal effect on changing the localization and structure of proteins.

Using machine learning models, it was shown that subcellular information allows better predictivity of single-cell drug perturbation states. To better incorporate spatial information into the predictive model the single-cell PPI events were transformed into graphs, and scPPI-GCN, a predictive pipeline, was developed for determining the drug treatment outcome from single-cell PPI data. The scPPIGCN predictive pipeline was benchmarked using spatial PPI graphs with ML, MLP, and MIL models and showed that spatial information plays an important role in improving the prediction of single-cell states. It was also shown that 3D graphs of PPI resulted in better cell state predictive abilities compared to 2D graphs of PPI.

To select the PPI of interest, a bioluminescence resonance energy transfer (BRET) based differential PPI discovery platform can be performed to reveal the differential interactions between wildtype NSCLC and EGFRm NSCLC cells. This quantitative high-throughput PPI screening platform can detect direct PPI with a proximity of <10 nm³⁷. By comparing the PPI between wild-type and mutant cells, mutation-directed novel PPIs can be discovered which function in potential alternative signaling pathways. The selected PPIs are involved in the EGFRm-related signaling pathways. The selection includes the upstream, downstream, and effectors in the AKT/mTOR, ERK/MEK, and YAP/TEAD1 pathways. They not only indicate the activity of the signaling pathway, but they also demonstrate the cell response including cell proliferation and apoptosis in response to drug perturbations. The selection of PPIs in the panel is based on the literature review, the OncoPPi, and Bioplex interactome networks.

Another method to determine potential PPIs is to prescreen interesting pairs using affinity purification-mass spectrometry. The underlying bait-prey pairs can be selectively purified and quantified by mass spectrometry analysis. This can help determine the interactome of target proteins between mutant and wide-type cells to understand the EGFRm-resistant mechanisms. The potential target proteins include p-AKT, ERK, and YAP. Due to the unknown biological questions involved in the signaling cascades, it is hard to determine one pair of proteins that do not interact with each other at all. To examine the specificity of RapMPI, protein knockout could be used as a negative control.

The results discussed herein exhibited an image-based approach to detect multiple PPI events and signaling markers in both cell culture and tissues. The conventional PPI detection approaches using MS cause the loss of spatial information due to the sample preparation step and peptide extraction³⁸. Unlike coimmunoprecipitation (Co-IP), PLA preserves the spatial information of proteins without cell lysis and can be performed on both cell cultures and tissues. The efficiency of detection can be improved using UnFold probes, which prevent cross-reactive detection of irrelevant proteins using a hairpin loop structure¹².

In conclusion, RapMPI illustrates the feasibility of multiplexing PLA and detecting multiple PPI distributions at the subcellular level. It also demonstrates the value of modeling drug treatment outcomes with graph-based PPI inputs, which integrates both the quantification and spatial information of protein interactions. This predictive model would predict patients' treatment outcomes with the signaling network inputs, which overcomes the limitation of lack of spatial details of signaling pathways using bulk signaling assays.

The following patents, applications and publications as listed below and throughout this document are hereby incorporated by reference in their entirety herein.

### REFERENCE LIST FOR EXAMPLES 2-4

1. Stewart, D. J. Wnt signaling pathway in non-small cell lung cancer. J Natl Cancer Inst 106, djt356 (2014).
2. Hommura, F. et al. Increased expression of β-catenin predicts better prognosis in nonsmall cell lung carcinomas. Cancer 94, 752-758 (2002).
3. Ramalingam, S. S. et al. Overall Survival with Osimertinib in Untreated, EGFR-Mutated Advanced NSCLC. New England Journal of Medicine 382, 41-50 (2020).
4. Leonetti, A. et al. Resistance mechanisms to Osimertinib in EGFR-mutated non-small cell lung cancer. British Journal of Cancer 121, 725-737 (2019).
5. Shi, P. et al. Overcoming Acquired Resistance to AZD9291, A Third-Generation EGFR Inhibitor, through Modulation of MEK/ERK-Dependent Bim and Mcl-1 Degradation. Clin Cancer Res 23, 6567-6579 (2017).
6. Li, Y. et al. ERK inhibition effectively overcomes acquired resistance of epidermal growth factor receptor-mutant non-small cell lung cancer cells to Osimertinib. Cancer 126, 1339-1350 (2020).
7. Gu, J. et al. Inhibition of ACK1 delays and overcomes acquired resistance of EGFR mutant NSCLC cells to the third generation EGFR inhibitor, Osimertinib. Lung Cancer 150, 26-35 (2020).
8. Gu, J. et al. MEK or ERK inhibition effectively abrogates emergence of acquired Osimertinib resistance in the treatment of epidermal growth factor receptor-mutant lung cancers. Cancer 126, 3788-3799 (2020).
9. Zhu, L. et al. Targeting c-Myc to Overcome Acquired Resistance of EGFR Mutant NSCLC Cells to the Third-Generation EGFR Tyrosine Kinase Inhibitor, Osimertinib. Cancer Res 81, 4822-4834 (2021).
10. Kurppa, K. J. et al. Treatment-Induced Tumor Dormancy through YAP-Mediated Transcriptional Reprogramming of the Apoptotic Pathway. Cancer Cell 37, 104-122.e12 (2020).
11. Kashima, Y. et al. Single-Cell Analyses Reveal Diverse Mechanisms of Resistance to EGFR Tyrosine Kinase Inhibitors in Lung Cancer. Cancer Res 81, 4835-4848 (2021).
12. Hu, Y., Peng, T., Gao, L. & Tan, K. CytoTalk: De novo construction of signal transduction networks using single-cell transcriptomic data. Science Advances (2021) doi:10.1126/sciadv.abf1356.
13. Gocho, Y. et al. Network-based systems pharmacology reveals heterogeneity in LCK and BCL2 signaling and therapeutic sensitivity of T-cell acute lymphoblastic leukemia. Nat Cancer 2, 284-299 (2021).
14. Han, L. et al. Single cell transcriptomics identifies a signaling network coordinating endoderm and mesoderm diversification during foregut organogenesis. Nat Commun 11, 4158 (2020).
15. Sufi, J. et al. Multiplexed single-cell analysis of organoid signaling networks. Nat Protoc 16, 4897-4918 (2021).
16. Bendall, S. C. et al. Single-Cell Mass Cytometry of Differential Immune and Drug Responses Across a Human Hematopoietic Continuum. Science 332, 687-696 (2011).
17. Leelatian, N., Diggins, K. E. & Irish, J. M. Characterizing Phenotypes and Signaling Networks of Single Human Cells by Mass Cytometry. Methods Mol Biol 1346, 99-113 (2015).
18. Linghu, C. et al. Spatial Multiplexing of Fluorescent Reporters for Imaging Signaling Network Dynamics. Cell 183, 1682-1698.e24 (2020).
19. Yang, J.-M. et al. Deciphering cell signaling networks with massively multiplexed biosensor barcoding. Cell 184, 6193-6206.e14 (2021).
20. Jones, S. et al. Core Signaling Pathways in Human Pancreatic Cancers Revealed by Global Genomic Analyses. Science 321, 1801-1806 (2008).
21. Mortazavi, A., Williams, B. A., McCue, K., Schaeffer, L. & Wold, B. Mapping and quantifying mammalian transcriptomes by RNA-Seq. Nature Methods 5, 621-628 (2008).
22. Blagoev, B. et al. A proteomics strategy to elucidate functional protein-protein interactions applied to EGF signaling. Nature Biotechnology 21, 315-318 (2003).
23. Powell, A. A. et al. Single cell profiling of circulating tumor cells: transcriptional heterogeneity and diversity from breast cancer cell lines. PLoS ONE 7, e33788 (2012).
24. Bendall, S. C. et al. Single-cell trajectory detection uncovers progression and regulatory coordination in human B cell development. Cell 157, 714-725 (2014).
25. Krutzik, P. O. & Nolan, G. P. Fluorescent cell barcoding in flow cytometry allows high-throughput drug screening and signaling profiling. Nature Methods 3, 361-368 (2006).
26. Huang, B., Jones, S. A., Brandenburg, B. & Zhuang, X. Whole-cell 3D STORM reveals interactions between cellular structures with nanometer-scale resolution. Nature Methods 5, 1047 (2008).
27. Hein, B., Willig, K. I. & Hell, S. W. Stimulated emission depletion (STED) nanoscopy of a fluorescent protein-labeled organelle inside a living cell. PNAS 105, 14271-14276 (2008).
28. Jungmann, R. et al. Multiplexed 3D cellular super-resolution imaging with DNA-PAINT and Exchange-PAINT. Nature Methods 11, 313-318 (2014).
29. Rust, M. J., Bates, M. & Zhuang, X. Sub-diffraction-limit imaging by stochastic optical reconstruction microscopy (STORM). Nature Methods 3, 793-796 (2006).
30. Giesen, C. et al. Highly multiplexed imaging of tumor tissues with subcellular resolution by mass cytometry. Nature Methods 11, 417-422 (2014).
31. Keren, L. et al. MIBI-TOF: A multiplexed imaging platform relates cellular phenotypes and tissue structure. Science Advances 5, eaax5851 (2019).
32. Goltsev, Y. et al. Deep Profiling of Mouse Splenic Architecture with CODEX Multiplexed Imaging. Cell 174, 968-981.e15 (2018).
33. Schürch, C. M. et al. Coordinated Cellular Neighborhoods Orchestrate Antitumoral Immunity at the Colorectal Cancer Invasive Front. Cell 0, (2020).
34. Gerdes, M. J. et al. Highly multiplexed single-cell analysis of formalin-fixed, paraffin-embedded cancer tissue. Proc Natl Acad Sci USA 110, 11982-11987 (2013).
35. Lin, J.-R., Fallahi-Sichani, M. & Sorger, P. K. Highly multiplexed imaging of single cells using a high-throughput cyclic immunofluorescence method. Nature Communications 6, ncomms9390 (2015).
36. Gut, G., Herrmann, M. D. & Pelkmans, L. Multiplexed protein maps link subcellular organization to cellular states. Science 361, (2018).
37. Ko, J., Oh, J., Ahmed, M. S., Carlson, J. C. T. & Weissleder, R. Ultra-fast Cycling for Multiplexed Cellular Fluorescence Imaging. Angewandte Chemie 132, 6906-6913 (2020).
38. Lin, J.-R. et al. Highly multiplexed immunofluorescence imaging of human tissues and tumors using t-CyCIF and conventional optical microscopes. eLife 7, e31657 (2018).
39. Li, Z. et al. The OncoPPi network of cancer-focused protein-protein interactions to inform biological insights and therapeutic strategies. Nature Communications 8, 14356 (2017).
40. Xu, J., Ma, H. & Liu, Y. Stochastic optical reconstruction microscopy (STORM). Curr Protoc Cytom 81, 12.46.1-12.46.27 (2017).
41. Gustafsson, N. et al. Fast live-cell conventional fluorophore nanoscopy with ImageJ through super-resolution radial fluctuations. Nature Communications 7, 12471 (2016).
42. Fredriksson, S. Visualizing signal transduction pathways by quantifying protein-protein interactions in native cells and tissue. Nat Methods 6, i-ii (2009).
43. Söderberg, O. et al. Direct observation of individual endogenous protein complexes in situ by proximity ligation. Nat Methods 3, 995-1000 (2006).
44. Coskun, A. F. & Cai, L. Dense transcript profiling in single cells by image correlation decoding. Nat Methods 13, 657-660 (2016).
45. La Manno, G. et al. RNA velocity of single cells. Nature 560, 494-498 (2018).
46. Coskun, A. F. & Cai, L. Dense transcript profiling in single cells by image correlation decoding l. Nature Methods 13, 657-660 (2016).
47. Lubeck, E., Coskun, A. F., Zhiyentayev, T., Ahmad, M. & Cai, L. Single-cell in situ RNA profiling by sequential hybridization l. Nature Methods 11, 360-361 (2014).
48. Ganesh, S. et al. Spatially resolved 3D metabolomic profiling in tissues. Science Advances 7, eabd0957 (2021).
49. Coskun, A. F. et al. Nanoscopic subcellular imaging enabled by ion beam tomography. Nature Communications 12, 789 (2021).
50. Leal, T. A. & Ramalingam, S. S. Immunotherapy in previously treated non-small cell lung cancer (NSCLC). J Thorac Dis 10, S422-S432 (2018).
51. Steffen McLouth, L. E. et al. Patient-reported tolerability of veliparib combined with cisplatin and etoposide for treatment of extensive stage small cell lung cancer: Neurotoxicity and adherence data from the ECOG ACRIN cancer research group E2511 phase II randomized trial. Cancer Med 9, 7511-7523 (2020).
52. Kamphorst, A. O. et al. Rescue of exhausted CD8 T cells by PD-1-targeted therapies is CD28-dependent. Science 355, 1423-1427 (2017).
53. Ma, W., Su, K. & Wu, H. Evaluation of some aspects in supervised cell type identification for single-cell RNA-seq: classifier, feature selection, and reference construction. Genome Biology 22, 264 (2021).
54. Lowe, M. C. et al. Winship 4851-19: A pilot study of neoadjuvant and adjuvant cemiplimab for high-risk cutaneous squamous cell carcinoma. JCO 39, TPS9593-TPS9593 (2021).
55. Lun, X.-K. & Bodenmiller, B. Profiling Cell Signaling Networks at Single-cell Resolution *. Molecular & Cellular Proteomics 19, 744-756 (2020).
56. Zhang, X. et al. Alterations in the Global Proteome and Phosphoproteome in Third Generation EGFR TKI Resistance Reveal Drug Targets to Circumvent Resistance. Cancer Res 81, 3051-3066 (2021).
57. Ando, T. et al. EGFR Regulates the Hippo pathway by promoting the tyrosine phosphorylation of MOB1. Commun Biol 4, 1-15 (2021).
58. Nguyen, C. D. K. & Yi, C. YAP/TAZ Signaling and Resistance to Cancer Therapy. Trends in Cancer 5, 283-296 (2019).
59. Liu, X., Yue, P., Zhou, Z., Khuri, F. R. & Sun, S.-Y. Death receptor regulation and celecoxib-induced apoptosis in human lung cancer cells. J Natl Cancer Inst 96, 1769-1780 (2004).
60. Lee, H.-W. et al. Profiling of protein-protein interactions via single-molecule techniques predicts the dependence of cancers on growth-factor receptors. Nat Biomed Eng 2, 239-253 (2018).
61. Kaushik, S. et al. A tyrosine kinase protein interaction map reveals targetable EGFR network oncogenesis in lung cancer. 2020.07.02.185173 https://www.biorxiv.org/content/10.1101/2020.07.02.185173v1 (2020) doi:10.1101/2020.07.02.185173.
62. Holland, D. O. & Johnson, M. E. Stoichiometric balance of protein copy numbers is measurable and functionally significant in a protein-protein interaction network for yeast endocytosis. PLOS Computational Biology 14, e1006022 (2018).
63. Maculins, T. et al. Discovery of Protein-Protein Interaction Inhibitors by Integrating Protein Engineering and Chemical Screening Platforms. Cell Chem Biol 27, 1441-1451.e7 (2020).
64. Dacic, S. et al. Significance of EGFR protein expression and gene amplification in non-small cell lung carcinoma. Am J Clin Pathol 125, 860-865 (2006).
65. Gray, J. E. et al. Tissue and Plasma EGFR Mutation Analysis in the FLAURA Trial: Osimertinib versus Comparator EGFR Tyrosine Kinase Inhibitor as First-Line Treatment in Patients with EGFR-Mutated Advanced Non-Small Cell Lung Cancer. Clin Cancer Res 25, 6644-6652 (2019).
66. Mei, X. D., Su, H., Song, J. & Dong, L. Prognostic significance of β-catenin expression in patients with non-small cell lung cancer: a meta-analysis. Biosci Trends 7, 42-49 (2013).
67. Jamalabadi, H., Alizadeh, S., Schönauer, M., Leibold, C. & Gais, S. Multivariate classification of neuroimaging data with nested subclasses: Biased accuracy and implications for hypothesis testing. PLOS Computational Biology 14, e1006486 (2018).
68. Misaki, M., Kim, Y., Bandettini, P. & Kriegeskorte, N. Comparison of multivariate classifiers and response normalizations for pattern-information fMRI. Neurolmage (2010) doi:10.1016/j.neuroimage.2010.05.051.

### REFERENCE LIST FOR EXAMPLE 1A, EXAMPLES 5-9

Alam, M. S. (2018). Proximity Ligation Assay (PLA). Curr Protoc Immunol 123, e58. https://doi.org/10.1002/cpim.58.
Baharlou, H., Canete, N. P., Cunningham, A. L., Harman, A. N., and Patrick, E. (2019). Mass Cytometry Imaging for the Study of Human Diseases—Applications and Data Analysis Strategies. Front Immunol 10, 2657. https://doi.org/10.3389/fimmu.2019.02657.
Bai, M., Li, W., Yu, N., Zhang, H., Long, F., and Zeng, A. (2017). The crosstalk between (3-catenin signaling and type I, type II and type III interferons in lung cancer cells. Am J Transl Res 9, 2788-2797.
Barldsi, F., Giaccone, G., Gallegos-Ruiz, M. I., Loundou, A., Span, S. W., Lefesvre, P., Kruyt, F. A. E., and Rodriguez, J. A. (2007). Global Histone Modifications Predict Prognosis of Resected Non-Small-Cell Lung Cancer. JCO 25, 4358-4364. https://doi.org/10.1200/JCO.2007.11.2599.
Brand, T. M., Iida, M., Li, C., and Wheeler, D. L. (2011). The Nuclear Epidermal Growth Factor Receptor Signaling Network and its Role in Cancer. Discov Med 12, 419-432.
Brand, T. M., Iida, M., Luthar, N., Starr, M. M., Huppert, E. J., and Wheeler, D. L. (2013). Nuclear EGFR as a Molecular Target in Cancer. Radiother Oncol 108, 10.1016/j.radonc.2013.06.010. https://doi.org/10.1016/j.radonc.2013.06.010.
Bray, M.-A., Singh, S., Han, H., Davis, C. T., Borgeson, B., Hartland, C., Kost-Alimova, M., Gustafsdottir, S. M., Gibson, C. C., and Carpenter, A. E. (2016). Cell Painting, a high-content image-based assay for morphological profiling using multiplexed fluorescent dyes. Nat Protoc 11, 1757-1774. https://doi.org/10.1038/nprot.2016.105.
Bu, J., Zhang, P., Zhu, K., Yan, Y., Shi, B., Wang, J., and Xu, S. (2020). Constructing a global transcriptional regulatory landscape for early non-small cell lung cancer to identify hub genes and key pathways. Aging (Albany NY) 12, 17948-17957. https://doi.org/10.18632/aging.103475.
Caicedo, A. (2013). PARACRINE AND AUTOCRINE INTERACTIONS IN THE HUMAN ISLET: MORE THAN MEETS THE EYE. Semin Cell Dev Biol 24, 11-21. https://doi.org/10.1016/j.semcdb.2012.09.007.
Chen, S., Guttridge, D. C., You, Z., Zhang, Z., Fribley, A., Mayo, M. W., Kitajewski, J., and Wang, C.-Y. (2001). WNT-1 Signaling Inhibits Apoptosis by Activating β-Catenin/T Cell Factor-Mediated Transcription. J Cell Biol 152, 87-96.
Cheng, H., Shcherba, M., Pendurti, G., Liang, Y., Piperdi, B., and Perez-Soler, R. (2014). Targeting the PI3K/AKT/mTOR pathway: potential for lung cancer treatment. Lung Cancer Manag 3, 67-75. https://doi.org/10.2217/lmt.13.72.
Chung, G. G., Provost, E., Kielhorn, E. P., Charette, L. A., Smith, B. L., and Rimm, D. L. (2001). Tissue Microarray Analysis of β-Catenin in Colorectal Cancer Shows Nuclear Phospho-β-catenin Is Associated with a Better Prognosis. Clin Cancer Res 7, 4013-4020.
Civenni, G., Holbro, T., and Hynes, N. E. (2003). de. EMBO Rep 4, 166-171. https://doi.org/10.1038/sj.embor.embor735.
Costes, S. V., Daelemans, D., Cho, E. H., Dobbin, Z., Pavlakis, G., and Lockett, S. (2004). Automatic and Quantitative Measurement of Protein-Protein Colocalization in Live Cells. Biophysical Journal 86, 3993-4003. https://doi.org/10.1529/biophysj.103.038422.
De Las Rivas, J., and Fontanillo, C. (2012). Protein-protein interaction networks: unraveling the wiring of molecular machines within the cell. Briefings in Functional Genomics 11, 489-496. https://doi.org/10.1093/bfgp/els036.
Duda, P., Akula, S. M., Abrams, S. L., Steelman, L. S., Martelli, A. M., Cocco, L., Ratti, S., Candido, S., Libra, M., Montalto, G., et al. (2020). Targeting GSK3 and Associated Signaling Pathways Involved in Cancer. Cells 9, 1110. https://doi.org/10.3390/cells9051110.
Elphick, L. M., Lee, S. E., Gouverneur, V., and Mann, D. J. (2007). Using Chemical Genetics and ATP Analogues To Dissect Protein Kinase Function. ACS Chem. Biol. 2, 299-314. https://doi.org/10.1021/cb700027u.
Erickson, H. P. (2009). Size and Shape of Protein Molecules at the Nanometer Level Determined by Sedimentation, Gel Filtration, and Electron Microscopy. Biol Proced Online 11, 32-51. https://doi.org/10.1007/s12575-009-9008-x.
Faivre, E. J., and Lange, C. A. (2007). Progesterone Receptors Upregulate Wnt-1 To Induce Epidermal Growth Factor Receptor Transactivation and c-Src-Dependent Sustained Activation of Erk1/2 Mitogen-Activated Protein Kinase in Breast Cancer Cells. Mol Cell Biol 27, 466-480. https://doi.org/10.1128/MCB.01539-06.
Fang, D., Hawke, D., Zheng, Y., Xia, Y., Meisenhelder, J., Nika, H., Mills, G. B., Kobayashi, R., Hunter, T., and Lu, Z. (2007). Phosphorylation of β-Catenin by AKT Promotes β-Catenin Transcriptional Activity *. Journal of Biological Chemistry 282, 11221-11229. https://doi.org/10.1074/jbc.M611871200.
Gautschi, O., Ratschiller, D., Gugger, M., Betticher, D. C., and Heighway, J. (2007). Cyclin D1 in non-small cell lung cancer: A key driver of malignant transformation. Lung Cancer 55, 1-14. https://doi.org/10.1016/j.lungcan.2006.09.024.
Giesen, C., Wang, H. A. O., Schapiro, D., Zivanovic, N., Jacobs, A., Hattendorf, B., Schuffler, P. J., Grolimund, D., Buhmann, J. M., Brandt, S., et al. (2014). Highly multiplexed imaging of tumor tissues with subcellular resolution by mass cytometry. Nat Methods 11, 417-422. https://doi.org/10.1038/nmeth.2869.
Gladden, A. B., and Diehl, J. A. (2005). Location, location, location: The role of cyclin D1 nuclear localization in cancer. Journal of Cellular Biochemistry 96, 906-913. https://doi.org/10.1002/jcb.20613.
Gu, J., Yao, W., Shi, P., Zhang, G., Owonikoko, T. K., Ramalingam, S. S., and Sun, S.-Y. (2020a). MEK or ERK inhibition effectively abrogates emergence of acquired Osimertinib resistance in the treatment of epidermal growth factor receptor-mutant lung cancers. Cancer 126, 3788-3799. https://doi.org/10.1002/cncr.32996.
Gu, J., Qian, L., Zhang, G., Mahajan, N. P., Owonikoko, T. K., Ramalingam, S. S., and Sun, S.-Y. (2020b). Inhibition of ACK1 delays and overcomes acquired resistance of EGFR mutant NSCLC cells to the third generation EGFR inhibitor, Osimertinib. Lung Cancer 150, 26-35. https://doi.org/10.1016/j.lungcan.2020.09.023.
Gu, J., Yao, W., Shi, P., Zhang, G., Owonikoko, T. K., Ramalingam, S. S., and Sun, S.-Y. (2020c). MEK or ERK inhibition effectively abrogates emergence of acquired Osimertinib resistance in the treatment of EGFR-mutant lung cancers. Cancer 126, 3788-3799. https://doi.org/10.1002/cncr.32996.
Guo, Y., Yang, K., Harwalkar, J., Nye, J. M., Mason, D. R., Garrett, M. D., Hitomi, M., and Stacey, D. W. (2005). Phosphorylation of cyclin D1 at Thr 286 during S phase leads to its proteasomal degradation and allows efficient DNA synthesis. Oncogene 24, 2599-2612. https://doi.org/10.1038/sj.onc.1208326.
Gut, G., Herrmann, M. D., and Pelkmans, L. (2018). Multiplexed protein maps link subcellular organization to cellular states. Science 361. https://doi.org/10.1126/science.aar7042.
Han, L., Qiu, P., Zeng, Z., Jorgensen, J. L., Mak, D. H., Burks, J. K., Schober, W., McQueen, T. J., Cortes, J., Tanner, S. D., et al. (2015). Single-cell mass cytometry reveals intracellular survival/proliferative signaling in FLT3-ITD-mutated AML stem/progenitor cells. Cytometry A 87, 346-356. https://doi.org/10.1002/cyto.a.22628.
Hao, H.-X., Jiang, X., and Cong, F. (2016). Control of Wnt Receptor Turnover by R-spondin-ZNRF3/RNF43 Signaling Module and Its Dysregulation in Cancer. Cancers (Basel) 8, 54. https://doi.org/10.3390/cancers8060054.
He, B., You, L., Uematsu, K., Xu, Z., Lee, A. Y., Matsangou, M., McCormick, F., and Jablons, D. M. (2004). A Monoclonal Antibody against Wnt-1 Induces Apoptosis in Human Cancer Cells. Neoplasia 6, 7-14.
He, J., Zhang, N., Zhu, Y., Jin, R., and Wu, F. (2021). MSC spheroids-loaded collagen hydrogels simultaneously promote neuronal differentiation and suppress inflammatory reaction through PI3K-Akt signaling pathway. Biomaterials 265, 120448. https://doi.org/10.1016/j.biomaterials.2020.120448.
Hommura, F., Furuuchi, K., Yamazaki, K., Ogura, S., Kinoshita, I., Shimizu, M., Moriuchi, T., Katoh, H., Nishimura, M., and Dosaka-Akita, H. (2002). Increased expression of β-catenin predicts better prognosis in nonsmall cell lung carcinomas. Cancer 94, 752-758. https://doi.org/10.1002/cncr.10213.
Hoshino, M., Fukui, H., Ono, Y., Sekikawa, A., Ichikawa, K., Tomita, S., Imai, Y., Imura, J., Hiraishi, H., and Fujimori, T. (2007). Nuclear Expression of Phosphorylated EGFR Is Associated with Poor Prognosis of Patients with Esophageal Squamous Cell Carcinoma. PAT 74, 15-21. https://doi.org/10.1159/000101047.
Hunt, K. K., Karakas, C., Ha, M. J., Biernacka, A., Yi, M., Sahin, A. A., Adjapong, O., Hortobagyi, G. N., Bondy, M. L., Thompson, P. A., et al. (2017). Cytoplasmic Cyclin E Predicts Recurrence in Patients with Breast Cancer. Clin Cancer Res 23, 2991-3002. https://doi.org/10.1158/1078-0432.CCR-16-2217.
Hwang, H. C., and Clurman, B. E. (2005). Cyclin E in normal and neoplastic cell cycles. Oncogene 24, 2776-2786. https://doi.org/10.1038/sj.onc.1208613.
Jin, J., Zhan, P., Katoh, M., Kobayashi, S. S., Phan, K., Qian, H., Li, H., Wang, X., Wang, X., and Song, Y. (2017). Prognostic significance of β-catenin expression in patients with non-small cell lung cancer: a meta-analysis. Transl Lung Cancer Res 6, 97-108. https://doi.org/10.21037/tlcr.2017.02.07.
Jin, S., Guerrero-Juarez, C. F., Zhang, L., Chang, I., Ramos, R., Kuan, C.-H., Myung, P., Plikus, M. V., and Nie, Q. (2021). Inference and analysis of cell-cell communication using CellChat. Nat Commun 12, 1088. https://doi.org/10.1038/s41467-021-21246-9.
Jouhilahti, E.-M., Peltonen, S., and Peltonen, J. (2008). Class III β-Tubulin Is a Component of the Mitotic Spindle in Multiple Cell Types. J Histochem Cytochem 56, 1113-1119. https://doi.org/10.1369/jhc.2008.952002.
Kagey, M. H., and He, X. (2017). Rationale for targeting the Wnt signalling modulator Dickkopf-1 for oncology. Br J Pharmacol 174, 4637-4650. https://doi.org/10.1111/bph.13894.
Kase, S., Sugio, K., Yamazaki, K., Okamoto, T., Yano, T., and Sugimachi, K. (2000). Expression of E-cadherin and β-Catenin in Human Non-Small Cell Lung Cancer and the Clinical Significance. Clin Cancer Res 6, 4789-4796.
Kielhorn, E., Provost, E., Olsen, D., D'Aquila, T. G., Smith, B. L., Camp, R. L., and Rimm, D. L. (2003). Tissue microarray-based analysis shows phospho-β-catenin expression in malignant melanoma is associated with poor outcome. International Journal of Cancer 103, 652-656. https://doi.org/10.1002/ijc.10893.
Knebel, A., Morrice, N., and Cohen, P. (2001). A novel method to identify protein kinase substrates: eEF2 kinase is phosphorylated and inhibited by SAPK4/p386. EMBO J 20, 4360-4369. https://doi.org/10.1093/emboj/20.16.4360.
Koutsami, M. K., Tsantoulis, P. K., Kouloukoussa, M., Apostolopoulou, K., Pateras, I. S., Spartinou, Z., Drougou, A., Evangelou, K., Kittas, C., Bartkova, J., et al. (2006). Centrosome abnormalities are frequently observed in non-small-cell lung cancer and are associated with aneuploidy and cyclin E overexpression. The Journal of Pathology 209, 512-521. https://doi.org/10.1002/path.2005.
Kurppa, K. J., Liu, Y., To, C., Zhang, T., Fan, M., Vajdi, A., Knelson, E. H., Xie, Y., Lim, K., Cejas, P., et al. (2020). Treatment-induced tumor dormancy through YAP-mediated transcriptional reprogramming of the apoptotic pathway. Cancer Cell 37, 104-122.e12. https://doi.org/10.1016/j.ccell.2019.12.006.
Leelatian, N., Diggins, K. E., and Irish, J. M. (2015). Characterizing Phenotypes and Signaling Networks of Single Human Cells by Mass Cytometry. Methods Mol Biol 1346, 99-113. https://doi.org/10.1007/978-1-4939-2987-0_8.
Leng, X., Wang, J., An, N., Wang, X., Sun, Y., and Chen, Z. (2020). Histone 3 lysine-27 demethylase KDM6A coordinates with KMT2B to play an oncogenic role in NSCLC by regulating H3K4me3. Oncogene 39, 6468-6479. https://doi.org/10.1038/s41388-020-01449-y.
Li, J. J., Bickel, P. J., and Biggin, M. D. (2014). System wide analyses have underestimated protein abundances and the importance of transcription in mammals. PeerJ 2, e270. https://doi.org/10.7717/peerj.270.
Li, V. S. W., Ng, S. S., Boersema, P. J., Low, T. Y., Karthaus, W. R., Gerlach, J. P., Mohammed, S., Heck, A. J. R., Maurice, M. M., Mahmoudi, T., et al. (2012). Wnt Signaling through Inhibition of β-Catenin Degradation in an Intact Axin1 Complex. Cell 149, 1245-1256. https://doi.org/10.1016/j.cell.2012.05.002.
Lin, J.-R., Fallahi-Sichani, M., and Sorger, P. K. (2015). Highly multiplexed imaging of single cells using a high-throughput cyclic immunofluorescence method. Nat Commun 6, 8390. https://doi.org/10.1038/ncomms9390.
Lin, J.-R., Fallahi-Sichani, M., Chen, J.-Y., and Sorger, P. K. (2016). Cyclic Immunofluorescence (CycIF), A Highly Multiplexed Method for Single-cell Imaging. Curr Protoc Chem Biol 8, 251-264. https://doi.org/10.1002/cpch.14.
Lin, J.-R., Izar, B., Wang, S., Yapp, C., Mei, S., Shah, P. M., Santagata, S., and Sorger, P. K. (2018). Highly multiplexed immunofluorescence imaging of human tissues and tumors using t-CyCIF and conventional optical microscopes. ELife 7, e31657. https://doi.org/10.7554/eLife.31657.
Lin, S.-Y., Makino, K., Xia, W., Matin, A., Wen, Y., Kwong, K. Y., Bourguignon, L., and Hung, M.-C. (2001). Nuclear localization of EGF receptor and its potential new role as a transcription factor. Nat Cell Biol 3, 802-808. https://doi.org/10.1038/ncb0901-802.
Liu, Z., Xing, D., Su, Q. P., Zhu, Y., Zhang, J., Kong, X., Xue, B., Wang, S., Sun, H., Tao, Y., et al. (2014). Super-resolution imaging and tracking of protein-protein interactions in sub-diffraction cellular space. Nat Commun 5, 4443. https://doi.org/10.1038/ncomms5443.
Lun, X.-K., and Bodenmiller, B. (2020). Profiling Cell Signaling Networks at Single-cell Resolution *. Molecular & Cellular Proteomics 19, 744-756. https://doi.org/10.1074/mcp.R119.001790.
Ma, B., and Hottiger, M. O. (2016). Crosstalk between Wnt/β-Catenin and NF-κB Signaling Pathway during Inflammation. Frontiers in Immunology 7, 378. https://doi.org/10.3389/fimmu.2016.00378.
Maurel, D., Comps-Agrar, L., Brock, C., Rives, M.-L., Bourrier, E., Ayoub, M. A., Bazin, H., Tinel, N., Durroux, T., Prdzeau, L., et al. (2008). Cell-surface protein-protein interaction analysis with time-resolved FRET and snap-tag technologies: application to GPCR oligomerization. Nat Methods 5, 561-567. https://doi.org/10.1038/nmeth.1213.
McCubrey, J. A., Steelman, L. S., Bertrand, F. E., Davis, N. M., Sokolosky, M., Abrams, S. L., Montalto, G., D'Assoro, A. B., Libra, M., Nicoletti, F., et al. (2014a). GSK-3 as potential target for therapeutic intervention in cancer. Oncotarget 5, 2881-2911.
McCubrey, J. A., Steelman, L. S., Bertrand, F. E., Davis, N. M., Abrams, S. L., Montalto, G., D'Assoro, A. B., Libra, M., Nicoletti, F., Maestro, R., et al. (2014b). Multifaceted roles of GSK-3 and Wnt/β-catenin in hematopoiesis and leukemogenesis: opportunities for therapeutic intervention. Leukemia 28, 15-33. https://doi.org/10.1038/leu.2013.184.
Meng, L., Michaud, G. A., Merkel, J. S., Zhou, F., Huang, J., Mattoon, D. R., and Schweitzer, B. (2008). Protein kinase substrate identification on functional protein arrays. BMC Biotechnol 8, 22. https://doi.org/10.1186/1472-6750-8-22.
Mingueneau, M., Krishnaswamy, S., Spitzer, M. H., Bendall, S. C., Stone, E. L., Hedrick, S. M., Pe'er, D., Mathis, D., Nolan, G. P., and Benoist, C. (2014). Single-cell mass cytometry of TCR signaling: Amplification of small initial differences results in low ERK activation in NOD mice. PNAS 111, 16466-16471. https://doi.org/10.1073/pnas.1419337111.
Mok, J., Im, H., and Snyder, M. (2009). Global identification of protein kinase substrates by protein microarray analysis. Nat Protoc 4, 1820-1827. https://doi.org/10.1038/nprot.2009.194.
Nakopoulou, L., Mylona, E., Papadaki, I., Kavantzas, N., Giannopoulou, I., Markaki, S., and Keramopoulos, A. (2006). Study of phospho-β-catenin subcellular distribution in invasive breast carcinomas in relation to their phenotype and the clinical outcome. Mod Pathol 19, 556-563. https://doi.org/10.1038/modpathol.3800562.
Nishimura, Y., Yoshioka, K., Bereczky, B., and Itoh, K. (2008). Evidence for efficient phosphorylation of EGFR and rapid endocytosis of phosphorylated EGFR via the early/late endocytic pathway in a gefitinib-sensitive non-small cell lung cancer cell line. Molecular Cancer 7, 42. https://doi.org/10.1186/1476-4598-7-42.
Parker, T. W., Rudeen, A. J., and Neufeld, K. L. (2020). Oncogenic Serine 45-Deleted β-Catenin Remains Susceptible to Wnt Stimulation and APC Regulation in Human Colonocytes. Cancers (Basel) 12, 2114. https://doi.org/10.3390/cancers12082114.
Peng, T., Thorn, K., Schroeder, T., Wang, L., Theis, F. J., Marr, C., and Navab, N. (2017). A BaSiC tool for background and shading correction of optical microscopy images. Nat Commun 8, 14836. https://doi.org/10.1038/ncomms14836.
Princiotta, M. F., Finzi, D., Qian, S.-B., Gibbs, J., Schuchmann, S., Buttgereit, F., Bennink, J. R., and Yewdell, J. W. (2003). Quantitating Protein Synthesis, Degradation, and Endogenous Antigen Processing. Immunity 18, 343-354. https://doi.org/10.1016/S1074-7613(03)00051-7.
Prossomariti, A., Piazzi, G., Alquati, C., and Ricciardiello, L. (2020). Are Wnt/β-Catenin and PI3K/AKT/mTORC1 Distinct Pathways in Colorectal Cancer? Cellular and Molecular Gastroenterology and Hepatology 10, 491-506. https://doi.org/10.1016/j.jcmgh.2020.04.007.
Rajapakse, H. E., Gahlaut, N., Mohandessi, S., Yu, D., Turner, J. R., and Miller, L. W. (2010). Time-resolved luminescence resonance energy transfer imaging of protein-protein interactions in living cells. PNAS 107, 13582-13587. https://doi.org/10.1073/pnas.1002025107.
Ramalingam, S. S., Vansteenkiste, J., Planchard, D., Cho, B. C., Gray, J. E., Ohe, Y., Zhou, C., Reungwetwattana, T., Cheng, Y., Chewaskulyong, B., et al. (2019). Overall Survival with Osimertinib in Untreated, EGFR-Mutated Advanced NSCLC. New England Journal of Medicine https://doi.org/10.1056/NEJMoa1913662.
Ramana, C. V. (2019). Insights into Functional Connectivity in Mammalian Signal Transduction Pathways by Pairwise Comparison of Protein Interaction Partners of Critical Signaling Hubs.
Raz, D. J., He, B., Rosell, R., and Jablons, D. M. (2006). Current Concepts in Bronchioloalveolar Carcinoma Biology. Clin Cancer Res 12, 3698-3704. https://doi.org/10.1158/1078-0432.CCR-06-0457.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682. https://doi.org/10.1038/nmeth.2019.
Schlange, T., Matsuda, Y., Lienhard, S., Huber, A., and Hynes, N. E. (2007). Autocrine WNT signaling contributes to breast cancer cell proliferation via the canonical WNT pathway and EGFR transactivation. Breast Cancer Res 9, R63. https://doi.org/10.1186/bcr1769.
Schurch, C. M., Bhate, S. S., Barlow, G. L., Phillips, D. J., Noti, L., Zlobec, I., Chu, P., Black, S., Demeter, J., McIlwain, D. R., et al. (2020). Coordinated Cellular Neighborhoods Orchestrate Antitumoral Immunity at the Colorectal Cancer Invasive Front. Cell 182, 1341-1359.e19. https://doi.org/10.1016/j.cell.2020.07.005.
Shen, T., Chen, Z., Qiao, J., Sun, X., and Xiao, Q. (2019). Neutralizing monoclonal antibody against Dickkopf2 impairs lung cancer progression via activating NK cells. Cell Death Discov. 5, 1-12. https://doi.org/10.1038/s41420-019-0204-4.
Shi, S.-R., Liu, C., Pootrakul, L., Tang, L., Young, A., Chen, R., Cote, R. J., and Taylor, C. R. (2008). Evaluation of the Value of Frozen Tissue Section Used as “Gold Standard” for Immunohistochemistry. American Journal of Clinical Pathology 129, 358-366. https://doi.org/10.1309/7CXUYXT23E5AL8KQ.
Sidhu, S. S., Nawroth, R., Retz, M., Lemjabbar-Alaoui, H., Dasari, V., and Basbaum, C. (2010). EMMPRIN regulates the canonical Wnt/β-catenin signaling pathway, a potential role in accelerating lung tumorigenesis. Oncogene 29, 4145-4156. https://doi.org/10.1038/onc.2010.166.
Siegelin, M. D., and Borczuk, A. C. (2014). Epidermal growth factor receptor mutations in lung adenocarcinoma. Lab Invest 94, 129-137. https://doi.org/10.1038/labinvest.2013.147.
Sinnberg, T., Menzel, M., Ewerth, D., Sauer, B., Schwarz, M., Schaller, M., Garbe, C., and Schittek, B. (2011). β-Catenin Signaling Increases during Melanoma Progression and Promotes Tumor Cell Survival and Chemoresistance. PLoS One 6, e23429. https://doi.org/10.1371/journal.pone.0023429.
Stewart, D. J. (2010). TUMOR AND HOST FACTORS THAT MAY LIMIT EFFICACY OF CHEMOTHERAPY IN NON-SMALL CELL AND SMALL CELL LUNG CANCER. Crit Rev Oncol Hematol 75, 173-234. https://doi.org/10.1016/j.critrevonc.2009.11.006.
Stewart, D. J. (2014). Wnt Signaling Pathway in Non-Small Cell Lung Cancer. JNCI: Journal of the National Cancer Institute 106. https://doi.org/10.1093/jnci/djt356.
Stringer, C., Wang, T., Michaelos, M., and Pachitariu, M. (2021). Cellpose: a generalist algorithm for cellular segmentation. Nat Methods 18, 100-106. https://doi.org/10.1038/s41592-020-01018-x.
Takam Kamga, P., Bazzoni, R., Dal Collo, G., Cassaro, A., Tanasi, I., Russignan, A., Tecchio, C., and Krampera, M. (2021). The Role of Notch and Wnt Signaling in MSC Communication in Normal and Leukemic Bone Marrow Niche. Frontiers in Cell and Developmental Biology 8, 1653. https://doi.org/10.3389/fcell.2020.599276.
Tan, Y. H., Liu, M., Nolting, B., Go, J. G., Gervay-Hague, J., and Liu, G. (2008). A Nanoengineering Approach for Investigation and Regulation of Protein Immobilization. ACS Nano 2, 2374-2384. https://doi.org/10.1021/nn800508f.
Traag, V. A., Waltman, L., and van Eck, N.J. (2019). From Louvain to Leiden: guaranteeing well-connected communities. Sci Rep 9, 5233. https://doi.org/10.1038/s41598-019-41695-z.
Troiani, S., Uggeri, M., Moll, J., Isacchi, A., Kalisz, H. M., Rusconi, L., and Valsasina, B. (2005). Searching for Biomarkers of Aurora-A Kinase Activity: Identification of in Vitro Substrates through a Modified KESTREL Approach. J. Proteome Res. 4, 1296-1303. https://doi.org/10.1021/pr050018e.
Uhlen, M., Bandrowski, A., Carr, S., Edwards, A., Ellenberg, J., Lundberg, E., Rimm, D. L., Rodriguez, H., Hiltke, T., Snyder, M., et al. (2016). A proposal for validation of antibodies. Nat Methods 13, 823-827. https://doi.org/10.1038/nmeth.3995.
Ungefroren, H. (2021). Autocrine TGF-β in Cancer: Review of the Literature and Caveats in Experimental Analysis. Int J Mol Sci 22, 977. https://doi.org/10.3390/ijms22020977.
Walsh, C. J. (2012). The Structure of the Mitotic Spindle and Nucleolus during Mitosis in the Amebo-Flagellate Naegleria. PLoS One 7, e34763. https://doi.org/10.1371/journal.pone.0034763.
Westermarck, J., Ivaska, J., and Corthals, G. L. (2013). Identification of Protein Interactions Involved in Cellular Signaling. Molecular & Cellular Proteomics 12, 1752-1763. https://doi.org/10.1074/mcp.R113.027771.
Xiao, Q., Wu, J., Wang, W.-J., Chen, S., Zheng, Y., Yu, X., Meeth, K., Sahraei, M., Bothwell, A. L. M., Chen, L., et al. (2018). DKK2 imparts tumor immunity evasion through β-catenin-independent suppression of cytotoxic immune cell activation. Nat Med 24, 262-270. https://doi.org/10.1038/nm.4496.
Xing, S., Wallmeroth, N., Berendzen, K. W., and Grefen, C. (2016). Techniques for the Analysis of Protein-Protein Interactions in Vivol[OPEN]. Plant Physiol 171, 727-758. https://doi.org/10.1104/pp. 16.00470.
Xue, L., Wang, W.-H., Iliuk, A., Hu, L., Galan, J. A., Yu, S., Hans, M., Geahlen, R. L., and Tao, W. A. (2012). Sensitive kinase assay linked with phosphoproteomics for identifying direct kinase substrates. PNAS 109, 5615-5620. https://doi.org/10.1073/pnas.1119418109.
Zeng, H., Lu, B., Zamponi, R., Yang, Z., Wetzel, K., Loureiro, J., Mohammadi, S., Beibel, M., Bergling, S., Reece-Hoyes, J., et al. (2018). mTORC1 signaling suppresses Wnt/β-catenin signaling through DVL-dependent regulation of Wnt receptor FZD level. PNAS 115, E10362-E10369. https://doi.org/10.1073/pnas.1808575115.
Zhang, J., Zhang, X., Zhao, X., Jiang, M., Gu, M., Wang, Z., and Yue, W. (2017). DKK1 promotes migration and invasion of non-small cell lung cancer via β-catenin signaling pathway. Tumour Biol. 39, 1010428317703820. https://doi.org/10.1177/1010428317703820.
Zhang, Y., Sun, L., Gao, X., Guo, A., Diao, Y., and Zhao, Y. (2019). RNF43 ubiquitinates and degrades phosphorylated E-cadherin by c-Src to facilitate epithelial-mesenchymal transition in lung adenocarcinoma. BMC Cancer 19, 670. https://doi.org/10.1186/s12885-019-5880-1.
Zheng, H., Yang, Z., Xin, Z., Yang, Y., Yu, Y., Cui, J., Liu, H., and Chen, F. (2020). Glycogen synthase kinase-30: a promising candidate in the fight against fibrosis. Theranostics 10, 11737-11753. https://doi.org/10.7150/thno.47717.
Zhu, K., Deng, Y., Weng, G., Hu, D., Huang, C., Matsumoto, K., Nagayasu, T., Koji, T., Zheng, X., Jiang, W., et al. (2018). Analysis of H3K27me3 expression and DNA methylation at CCGG sites in smoking and non-smoking patients with non-small cell lung cancer and their clinical significance. Oncol Lett 15, 6179-6188. https://doi.org/10.3892/o1.2018.8100.

### REFERENCE LIST FOR EXAMPLE 1b, EXAMPLES 10-13

1. Gu, J. et al. MEK or ERK inhibition effectively abrogates emergence of acquired Osimertinib resistance in the treatment of EGFR-mutant lung cancers. Cancer 126, 3788-3799 (2020).
2. Cheng, H. et al. Targeting the PI3K/AKT/mTOR pathway: potential for lung cancer treatment. Lung Cancer Manag 3, 67-75 (2014).
3. Xin, X. et al. CD147/EMMPRIN overexpression and prognosis in cancer: A systematic review and meta-analysis. Sci Rep 6, 32804 (2016).
4. Kurppa, K. J. et al. Treatment-induced tumor dormancy through YAP-mediated transcriptional reprogramming of the apoptotic pathway. Cancer Cell 37, 104-122.e12 (2020).
5. Ando, T. et al. EGFR Regulates the Hippo pathway by promoting the tyrosine phosphorylation of MOB1. Commun Biol 4, 1-15 (2021).
6. Nguyen, C. D. K. & Yi, C. YAP/TAZ Signaling and Resistance to Cancer Therapy. Trends in Cancer 5, 283-296 (2019).
7. Kaushik, S. et al. A tyrosine kinase protein interaction map reveals targetable EGFR network oncogenesis in lung cancer. 2020.07.02.185173 Preprint at https://doi.org/10.1101/2020.07.02.185173 (2020).
8. Lee, H.-W. et al. Profiling of protein-protein interactions via single-molecule techniques predicts the dependence of cancers on growth-factor receptors. Nat Biomed Eng 2, 239-253 (2018).
9. Rajapakse, H. E. et al. Time-resolved luminescence resonance energy transfer imaging of protein-protein interactions in living cells. PNAS 107, 13582-13587 (2010).
10. Maurel, D. et al. Cell-surface protein-protein interaction analysis with time-resolved FRET and snap-tag technologies: application to GPCR oligomerization. Nat Methods 5, 561-567 (2008).
11. Jalili, R., Horecka, J., Swartz, J. R., Davis, R. W. & Persson, H. H. J. Streamlined circular proximity ligation assay provides high stringency and compatibility with low-affinity antibodies. Proceedings of the National Academy of Sciences 115, E925-E933 (2018).
12. Klaesson, A. et al. Improved efficiency of in situ protein analysis by proximity ligation using UnFold probes. Sci Rep 8, 5400 (2018).
13. Krieger, C. C., Boutin, A., Neumann, S. & Gershengorn, M. C. Proximity ligation assay to study TSH receptor homodimerization and crosstalk with IGF-1 receptors in human thyroid cells. Frontiers in Endocrinology 13, (2022).
14. Krzeptowski, W. et al. Proximity Ligation Assay Detection of Protein-DNA Interactions—Is There a Link between Heme Oxygenase-1 and G-quadruplexes? Antioxidants 10, 94 (2021).
15. Protocol for visualizing conditional interaction between transmembrane and cytoplasmic proteins. https://star-protocols.cell.com/protocols/558.
16. Vistain, L. et al. Quantification of extracellular proteins, protein complexes and mRNAs in single cells by proximity sequencing. Nat Methods 19, 1578-1589 (2022).
17. Söderberg, O. et al. Direct observation of individual endogenous protein complexes in situ by proximity ligation. Nat Methods 3, 995-1000 (2006).
18. Fredriksson, S. Visualizing signal transduction pathways by quantifying protein-protein interactions in native cells and tissue. Nat Methods 6, i-ii (2009).
19. Alam, M. S. Proximity Ligation Assay (PLA). Curr Protoc Immunol 123, e58 (2018).
20. Cai, S. et al. Multiplexed protein profiling reveals spatial subcellular signaling networks. IScience 25, 104980 (2022).
21. Shi, P. et al. Overcoming acquired resistance to AZD9291, a third generation EGFR inhibitor, through modulation of MEK/ERK-dependent Bim and Mcl-1 degradation. Clin Cancer Res 23, 6567-6579 (2017).
22. Willis, S. N. et al. Proapoptotic Bak is sequestered by Mcl-1 and Bcl-xL, but not Bcl-2, until displaced by BH3-only proteins. Genes Dev 19, 1294-1305 (2005).
23. Hwang, H. C. & Clurman, B. E. Cyclin E in normal and neoplastic cell cycles. Oncogene 24, 2776-2786 (2005).
24. Zhu, L. et al. Targeting c-Myc to overcome acquired resistance of EGFR mutant NSCLC cells to the third generation EGFR tyrosine kinase inhibitor, Osimertinib. Cancer Res 81, 4822-4834 (2021).
25. Li, J.-Q., Miki, H., Ohmori, M., Wu, F. & Funamoto, Y. Expression of cyclin E and cyclin-dependent kinase 2 correlates with metastasis and prognosis in colorectal carcinoma. Human Pathology 32, 945-953 (2001).
26. Xie, X., Shu, R., Yu, C., Fu, Z. & Li, Z. Mammalian AKT, the Emerging Roles on Mitochondrial Function in Diseases. Aging Dis 13, 157-174 (2022).
27. Topacio, B. R. et al. Cyclin D-Cdk4,6 Drives Cell-Cycle Progression via the Retinoblastoma Protein's C-Terminal Helix. Mol Cell 74, 758-770.e4 (2019).
28. Christian, F., Smith, E. L. & Carmody, R. J. The Regulation of NF-κB Subunits by Phosphorylation. Cells 5, 12 (2016).
29. Zhang, S., Xiong, X. & Sun, Y. Functional characterization of SOX2 as an anticancer target. Sig Transduct Target Ther 5, 1-17 (2020).
30. Li, L. et al. Protective autophagy decreases Osimertinib cytotoxicity through regulation of stem cell-like properties in lung cancer. Cancer Letters 452, 191-202 (2019).
31. Frank, D. O. et al. The Pro-Apoptotic BH3-Only Protein Bim Interacts with Components of the Translocase of the Outer Mitochondrial Membrane (TOM). PLoS One 10, e0123341 (2015).
32. Lalier, L. et al. TOM20-mediated transfer of Bcl2 from ER to MAM and mitochondria upon induction of apoptosis. Cell Death Dis 12, 1-11 (2021).
33. Lu, A. & Pfeffer, S. R. Golgi-associated RhoBTB3 targets Cyclin E for ubiquitylation and promotes cell cycle progression. J Cell Biol 203, 233-250 (2013).
34. Makhoul, C. & Gleeson, P. A. Regulation of mTORC1 activity by the Golgi apparatus. Fac Rev 10, 50 (2021).
35. Black, S. et al. CODEX multiplexed tissue imaging with DNA-conjugated antibodies. Nat Protoc 16, 3802-3835 (2021).
36. Hagey, D. W. & Muhr, J. Sox2 Acts in a Dose-Dependent Fashion to Regulate Proliferation of Cortical Progenitors. Cell Reports 9, 1908-1920 (2014).
37. Mo, X. et al. Systematic discovery of mutation-directed neo-protein-protein interactions in cancer. Cell 185, 1974-1985.e12 (2022).
38. Farooq, Q. ul A., Shaukat, Z., Aiman, S. & Li, C.-H. Protein-protein interactions: Methods, databases, and applications in virus-host study. World J Virol 10, 288-300 (2021).
39. Overview. ASHLAR https://labsyspharm.github.io/ashlar/overview/overview-land.html.
40. Stringer, C., Wang, T., Michaelos, M. & Pachitariu, M. Cellpose: a generalist algorithm for cellular segmentation. http://biorxiv.org/lookup/doi/10.1101/2020.02.02.931238 (2020) doi:10.1101/2020.02.02.931238.
41. Greenwald, N. F. et al. Whole-cell segmentation of tissue images with human-level performance using large-scale data annotation and deep learning. http://biorxiv.org/lookup/doi/10.1101/2021.03.01.431313 (2021) doi:10.1101/2021.03.01.431313.
42. Bannon, D. et al. DeepCell Kiosk: scaling deep learning-enabled cellular image analysis with Kubernetes. Nat Methods 18, 43-45 (2021).

1. A method of detecting multiple protein interactions within a cell or tissue sample, the method comprising:
a) applying to the cell or tissue sample at least a first targeting moiety and a second targeting moiety, wherein the first targeting moiety interacts with a first specific protein of interest and the second targeting moiety interacts with a second protein of interest in the cell or tissue sample, wherein an interaction between the first specific protein of interest and the second specific protein of interest causes the first targeting moiety and second targeting moiety to be in close proximity to each other, wherein when the first targeting moiety and the second targeting moiety are in close proximity to each other, at least one detectable signal is produced;
b) imaging the detectable signal or signals in the cell or tissue sample;
c) deactivating the detectable signal or signals;
d) repeating steps a), b), and c), wherein the first targeting moiety and the second targeting moiety are different in each repetition.

2. The method of claim 1, wherein each of the at least two targeting moieties comprises antibody and an oligonucleotide.

3. The method of claim 2, wherein producing a detectable signal comprises:
amplifying the oligonucleotides which are in close proximity to each other, wherein the oligonucleotides are amplified only when in close proximity to each other; and
applying to the cell or tissue sample a fluorescent-labeled oligonucleotide, wherein the fluorescent-labeled oligonucleotide is complementary to at least a part of the amplified oligonucleotides;
wherein the detectable signal comprises the fluorescent-labeled nucleotide coupled to at least a part of the amplified oligonucleotides.

4. The method of claim 3, wherein step c) comprises incubating the cell or tissue sample with a nuclease to remove any oligonucleotides.

5. The method of claim 4, wherein the nuclease is selected from DNase and Nuclease P1.

6. (canceled)

7. (canceled)

8. (canceled)

9. (canceled)

10. (canceled)

11. The method of claim 1, wherein step b) further comprises taking images of the cell or tissue sample at multiple spatial and/or temporal locations.

12. The method of claim 11, further comprising analysis of the images using a machine learning algorithm.

13. The method of claim 1, further comprising a step of detecting subcellular spatial protein networks in the cell or tissue sample.

14. The method of claim 1, further comprising:
e) applying to the cell or tissue sample at least one imaging moiety which can interact with a specific protein of interest in the cell or tissue sample, wherein when the at least one imaging moiety interacts with a specific protein of interest, it produces a detectable signal;
f) imaging the detectable signal or signals in the cell or tissue sample;
g) deactivating the detectable signal or signals;
h) repeating steps e), f), and g), wherein the at least one imaging moiety is different in each repetition.

15. The method of claim 14, wherein the at least one imaging moiety comprises an antibody.

16. The method of claim 15, wherein the antibody is preconjugated with a fluorescent dye, and wherein the detectable signal comprises the fluorescent dye.

17. (canceled)

18. The method of claim 14, wherein step g) comprises fluorescent bleaching.

19. (canceled)

20. The method of claim 14, wherein steps e), f), and g) are repeated at least three times.

21. (canceled)

22. The method of claim 14, wherein subcellular spatial protein networks in the cell or tissue sample are detected.

23. (canceled)

24-44. (canceled)

45. A system for multiplexed imaging comprising:
a computer program capable of receiving at least one parameter from a user;
a support for a cell or tissue sample;
at least one port for delivering fluids to the support for the cell or tissue sample; and
at least one port for aspirating fluids from the cell or tissue sample.

46. The system of claim 45, wherein each of the at least one port for delivering fluids and the at least one port for aspirating fluids further comprise a valve.

47. The system of claim 46, wherein each valve is controlled by the computer program.

48. The system of claim 45, wherein the system can be coupled to a microscope.

49. (canceled)

50. (canceled)

51. The system of claim 45, wherein each port further comprises a fluidic tube and a needle tip.

52. The system of claim 45, further comprising at least one port for preventing overflow using a vacuum.
